SEQUENCE LISTING (38 sequences)

SEQ ID NO: 1 - DNA, 2544 bp, Homo Sapien
  CDS (693)...(1877)
caccgggcagcgccgagggttgccggccggcgcgcggggagtagagggcgcgggccgcagtgccgggttccagagggagctctgcgccgggtccttccctgtggtagccccaggacacccccagcctcaacatcccattctgggactcctgccctgttcccacattcgttctacctcgagtctccaggagcttccagtggcttggtcaccgccaactctcgtccatgcctcttagagcccctttcccggcctcaccgggtgtcgcttaatagtcttgggaccttaaggagcaagtcagcccctgcggaccctcccagtgaagagaaagagctggctgtgcggtggaatttggaagagacgacgtttgggagcctttgctgagtccagggagagaggcgtcccccaccgtgccgctgcagctcgggcagagccgccaagctttgggttctctagggtgtgtacccaggagtggatctgctgtatcatatggtcactctattcaacctttcgagaaaccaccaaattgtttcttcaggaaatgcaccatctgacatccccactttatgaggatccccacgtctctgtcatctcaccaacacttgtgctgaggaacctctaatcatctcccatggatttgtgatcagcgttgcagctctcccagcagccctggacagtggcccccagcagtcagcatgtggctgccgcgcgtctccagcacagcagtgaccgcgctcctcctggcgcagaccttcctcctcctctttctggtttcccggccagggccctcgtccccagcaggcggcgaggcgcgcgtgcatgtgctggtgctgtcctcgtggcgctcgggctcgtccttcgtgggccaactcttcaaccagcaccccgacgtcttctacctaatggagcccgcgtggcacgtgtggaccaccctgtcgcagggcagcgccgcaacgctgcacatggctgtgcgcgacctggtgcgctccgtcttcctgtgcgacatggacgtgtttgatgcctatctgccttggcgccgcaacctgtccgacctcttccagtgggccgtgagccgtgcactgtgctcgccacccgcctgcagtgcctttccccgaggcgccatcagcagcgaggccgtgtgcaagccactgtgcgcgcggcagtccttcaccctggcccgggaggcctgccgctcctacagccacgtggtgctcaaggaggtgcgcttcttcaacctgcaggtgctctacccgctgctcagcgaccccgcgctcaacctacgcatcgtgcacctggtgcgcgacccgcgggccgtgctgcgctcccgggagcagacagccaaggctctggcgcgtgacaacggcatcgtgctgggcaccaacggcacgtgggtggaggccgaccccggcctgcgcgtggtgcgcgaggtgtgccgtagccacgtacgcatcgccgaggccgccacactcaagccgccaccctttctgcgcggccgctaccgcctggtgcgcttcgaggacctggcgcgggagccgctggcagaaatccgtgcgctctacgccttcactgggctcagtctcacgccacagctcgaggcctggatccataacatcacccacggatctggacctggtgcgcgccgcgaagccttcaagacttcgtccaggaatgcgctcaacgtctcccaggcctggcgccatgcgctgccctttgccaagatccgccgcgtgcaggaactgtgcgctggtgcgctgcagctgctgggctaccggcctgtgtactctgaggacgagcagcgcaacctcgcccttgatctggtgctgccacgaggcctgaacggcttcacttgggcatcatccaccgcctcgcacccccgaaattagtggaggccacagttgtagcaggcgctaggcccgggaggagagtgcatggtgcagagggggctggggcgcacggagaagcaggtccctatattgaccaaggagtttgtggtacgacccctccccctccccaagtaggcaaggactgcacgtttctttctctcttgattcttggttttcctttgagtcctcatttcagggttcacttcaggggctcccaaagcgacaagatcgttagggagagaggcccagggtggggactgggaatttaaggagagctgggaacggatcccttaggttcaggaagcttctgtgcaagctgcgaggatggcttgggccgaagggttgctctgcccgccgcgctagctgtgagctgagcaaagccctgggctcacagcaccccaaaagcctgtggcttcagtcctgcgtctgcaccacccaatcaaaaggatcgttttgttttgtttttaaagaaaggtgagattggcttggttcttcatgagcacatttgatatagctctttttctgtttttccttgctcatttcgttttggggaagaaatctgtactgtattgggattgtaaagaacatctttgcactcagacagtttacagaaataaatgttttttttgtttttcaaaaaaaaaaaaaaaaaaaaaaaaaaaaa

SEQ ID NO: 2 - protein, 395 aa, Homo Sapien
MWLPRVSSTAVTALLLAQTFLLLFLVSRPGPSSPAGGEARVHVLVLSSWRSGSSFVGQLFNQHPDVFYLMEPAWHVWTTLSQGSAATLHMAVRDLVRSVFLCDMDVFDAYLPWRRNLSDLFQWAVSRALCSPPACSAFPRGAISSEAVCKPLCARQSFTLAREACRSYSHVVLKEVRFFNLQVLYPLLSDPALNLRIVHLVRDPRAVLRSREQTAKALARDNGIVLGTNGTWVEADPGLRVVREVCRSHVRIAEAATLKPPPFLRGRYRLVRFEDLAREPLAEIRALYAFTGLSLTPQLEAWIHNITHGSGPGARREAFKTSSRNALNVSQAWRHALPFAKIRRVQELCAGALQLLGYRPVYSEDEQRNLALDLVLPRGLNGFTWASSTASHPRN

SEQ ID NO: 3 - protein, 395 aa, Artificial Sequence
  synthetic construct
MWLPRFSSTXVTXLLLAQTXLLLFLVSRPGPSSPAGGEXRVHVLVLSSWRSGSSFVGQLFSQHPDVFYLMEPAWHVWTTLSQGSAATLHMAVRDLXRSVFLCDMDVFDAYLPWRRNLSDLFQWAVSRALCSPPACSAFPRGXISSEXVCKPLCARQPFXLAREACRSYSHVVLKEVRFFNLQVLYPLLSDPALNLRIVHLVRDPRAVLRSREQTAKALARDNGIVLGTNGTWVEADPXLRVVREVCRSHVRIAEAATLKPPPFLRGRYRLVRFEDLAREPLAEIRALYAFTGLXLTPQLEAWIHNITHGSGPGARREAFKTSSRNALNVSQAWRHALPFAKIRRVQELCAGALQLLGYRPVYSEDEQRDLXLDLVLPRGXDXFXWASSTXXXPXX

SEQ ID NO: 4 - protein, 390 aa, Homo Sapien
MWLPRFSSKTVTVLLLAQTTCLLLFIISRPGPSSPAGGEDRVHVLVLSSWRSGSSFLGQLFSQHPDVFYLMEPAWHVWTTLSQGSAATLHMAVRDLMRSIFLCDMDVFDAYMPQSRNLSAFFNWATSRALCSPPACSAFPRGTISKQDVCKTLCTRQPFSLAREACRSYSHVVLKEVRFFNLQVLYPLLSDPALNLRIVHLVRDPRAVLRSREAAGPILARDNGIVLGTNGKWVEADPHLRLIREVCRSHVRIAEAATLKPPPFLRGRYRLVRFEDLAREPLAEIRALYAFTGLTLTPQLEAWIHNITHGSGIGKPIEAFHTSSRNARNVSQAWRHALPFTKILRVQEVCAGALQLLGYRPVYSADQQRDLTLDLVLPRGPDHFSWASPD

SEQ ID NO: 5 - protein, 418 aa, Mus musculus
SGSLCAPWVRSAEAQRAAQALARGMRLPRFSSTVMLSLLMVQTGILVFLVSRQVPSSPAGLGERVHVLVLSSWRSGSSFVGQLFSQHPDVFYLMEPAWHVWDTLSQGSAPALHMAVRDLIRSVFLCDMDVFDAYLPWRRNISDLFQWAVSRALCSPPVCEAFARGNISSEEVCKPLCATRPFGLAQEACSSYSHVVLKEVRFFNLQVLYPLLSDPALNLRIVHLVRDPRAVLRSREQTAKALARDNGIVLGTNGTWVEADPRLRVVNEVCRSHVRIAEALHKPPPFLQDRYRLVRYEDLARDPLTVIRELYAFTGLGLTPQLQTWIHNITHGSGPGARREAFKTTSRDALSVSQAWRHTLPFAKIRRVQELCGGALQLLGYRSVHSELEQRDLSLDLLLPRGMDSFKWASSTEKQPES

SEQ ID NO: 6 - protein, 169 aa, Homo Sapien
SPAGGEARVHVLVLSSWRSGSSFVGQLFNQHPDVFYLMEPAWHVWTTLSEACRSYSHVVLKEVRFFNLQVLYPLLSDPALNLRIVHLVRDPRAVLRSREQTAKALARDNEAATLKPPPFLRGRYRLVRFEDLAREPLAEIRALYAFTGLSLTPQLEAWIHNITHGSGPG

SEQ ID NO: 7 - protein, 169 aa, Homo Sapien
SPAGGEDRVHVLVLSSWRSGSSFLGQLFSQHPDVFYLMEPAWHVWTTLSEACRSYSHVVLKEVRFFNLQVLYPLLSDPALNLRIVHLVRDPRAVLRSREAAGPILARDNEAATLKPPPFLRGRYRLVRFEDLAREPLAEIRALYAFTGLTLTPQLEAWIHNITHGSGIG

SEQ ID NO: 8 - protein, 171 aa, Homo Sapien
SSLSMKAQPERMHVLVLSSWRSGSSFVGQLFGQHPDVFYLMEPAWHVWMTFKKACRSYSHVVLKEVRFFNLQSLYPLLKDPSLNLHIVHLVRDPRAVFRSRERTKGDLMIDSKTIQSLPKALQERYLLVRYEDLARAPVAQTSRMYEFVGLEFLPHLQTWVHNITRGKGMG

SEQ ID NO: 9 - protein, 169 aa, Homo Sapien
APEGVGDKRHWMYVFTTWRSGSSFFGELFNQNPEVFFLYEPVWHVWQKLYEECRKYRTLVIKGVRVFDVAVLAPLLRDPALDLKVIHLVRDPRAVASSRIRSRHGLIRESQTALQPPDWLQGHYLVVRYEDLVGDPVKTLRRVYDFVGLLVSPEMEQFALNMTSGSGSS

SEQ ID NO: 10 - protein, 179 aa, Homo Sapien
RLCEESPTFAYNLSRKTHILILATTRSGSSFVGQLFNQHLDVFYLFEPLYHVQNTLIPRFEACRERSHVAIKTVRVPEVNDLRALVEDPRLNLKVIQLVRDPRGILASRSETFRDTYRLWSTGLMRPPWLKGKYMLVRYEDLARNPMKKTEEIYGFLGIPLDSHVARWIQNNTRGDPTL

SEQ ID NO: 11 - protein, 174 aa, Homo Sapien
EEPPRPAVAGPRRHVLLMATTRTGSSFVGEFFNQQGNIFYLFEPLWHIERTVSFEEACRRKEHMALKAVRIRQLEFLQPLAEDPRLDLRVIQLVRDPRAVLASRMVAFAGKYKTWELGLRQPAWLRGRYMLVRYEDVARGPLQKAREMYPFAGIPLTPQVEDWIQKNTQAAHDG

SEQ ID NO: 12 - DNA, 20 bp, Homo Sapien
agagccgaaacctgtccgcc

SEQ ID NO: 13 - DNA, 21 bp, Homo Sapien
gcgtagagtgcgcggatctct

SEQ ID NO: 14 - DNA, 23 bp, Homo Sapien
tatctgccttggcgccgcaacct

SEQ ID NO: 15 - DNA, 23 bp, Homo Sapien
ccgttgtcacgcgccagagcctt

SEQ ID NO: 16 - DNA, 22 bp, Homo Sapien
cggggaaaggcactgcaggcgg

SEQ ID NO: 17 - DNA, 23 bp, Homo Sapien
cgaccccgcgctcaacctacgca

SEQ ID NO: 18 - DNA, 22 bp, Homo Sapien
cacgaggcctgaacggcttcac

SEQ ID NO: 19 - DNA, 22 bp, Homo Sapien
cgggcctagcgcctgctacaac

SEQ ID NO: 20 - DNA, 21 bp, Homo Sapien
gcccctaaccgctgcgctctc

SEQ ID NO: 21 - DNA, 20 bp, Homo Sapien
ggcttgcacacggcctcgct

SEQ ID NO: 22 - DNA, 26 bp, Homo Sapien
gacgtgtttgatgcctatctgccttg

SEQ ID NO: 23 - DNA, 18 bp, Homo Sapien
cggcgcgcaccaggtcca

SEQ ID NO: 24 - DNA, 21 bp, Homo Sapien
ctcccgggagcagacagccaa

SEQ ID NO: 25 - DNA, 18 bp, Homo Sapien
ctcccgggcctagcgcct

SEQ ID NO: 26 - DNA, 23 bp, Homo Sapien
tgctctacccgctgctcagcgac

SEQ ID NO: 27 - DNA, 23 bp, Homo Sapien
cgggagcgcagcacggcccccgg

SEQ ID NO: 28 - DNA, 21 bp, Homo Sapien
tgcccccagaaaagaatcaaa

SEQ ID NO: 29 - DNA, 20 bp, Homo Sapien
tcctcccaagtcccttggag

SEQ ID NO: 30 - DNA, 28 bp, Homo Sapien
ccacagaaggaaggacagagtaaatgaa

SEQ ID NO: 31 - DNA, 32 bp, Homo Sapien
ttccctttactattataaaaatgctgctaatg

SEQ ID NO: 32 - DNA, 31 bp, Homo Sapien
tgctgaatggctaactgaaggaatactatac

SEQ ID NO: 33 - DNA, 31 bp, Homo Sapien
catatcctgtctggcctaaaccttagtttac

SEQ ID NO: 34 - DNA, 23 bp, Homo Sapien
gggcacagacagagggaaaaacc

SEQ ID NO: 35 - DNA, 23 bp, Homo Sapien
ggccaagttcaggtcagcttcca

SEQ ID NO: 36 - DNA, 26 bp, Homo Sapien
agaccttcctcctcctctttctggtt

SEQ ID NO: 37 - DNA, 26 bp, Homo Sapien
ttggcccacgaaggacgagcccgggc

SEQ ID NO: 38 - DNA, 48436 bp, Homo Sapien
tctcaaaaataataataataataaaataataataataatagtctctctgatgtgttgactcccctgggaagtcttacatactcctatgcagccattctttataaatcaactggtctgttagggttagagtaacaaaaacatgaagaaagcataaagaatcattcaactaaggtgaaattgtgaagtcaatactgaaacaaaacaagtccattatgatggtgacaggaaatggagtcagggcccaaggttttggtcaatctctcaaaattgagaggctgaccaaaaggcagaaatgtttaaattcaattaaatttggcccaaagttcccagcactttgggaggccaaggtgggcggatcatgaggtcaggagatcgagaccatcttggccaacatggtgaaaccccatctctactaaaatacaaaaaattagccgggcatggtggcacatgcctataatcccagctactcgggaggctgaggcagaggaatcactagaacccaggaggcagaggttgcagtgagctgagatcgtgcactgcactccagcctggtgacagagcaagactccgtctccaaaaaaaaaaaaagagaatttggcccaaagctgctgccatacctgttgaactgcaacctaacttaatatttaagtaaactgcctcccaactgagactatattcttgtaacaaatagttgaatctcagcaagtcacagcagctgtgctttaaccagtcacaggctgccaactgatcagaccaagtccatataaggcaaatgctgagctgtaccccatcagactggtttctctgtgttacttccaaaaaattcggcctgccagtgtttctgggtggagcactttgaacctttactggttcagggtgctgcccgattcataaattttctttgctcaaataaagtctgcttaatttgtctaatgtttttctattaacagttcagatgacttggcctctacccaaactctttctttcccctagactctcctctcttggaatgcatcctgaagcagctgaaaaggggtgccccgggcccagcagggagcaaaatctggtgatattgcttctgaacatcccacatgtgccacacacgtgcacccccccacacacacacatgcacactcacatgcacactcacatgcacactcacatgcacactcacatgcacactcacatgcacactcacatgcacactcacatgcacacacagcctggactctgttccccttatgcccctggcaccacactccatcaaagccattgacctttatatccccctgtgtcttcagtaagaggtatatcaggccagacatggtggctcatccctgtaattatcaattacccggtctctggtattctgttacagcagcacaaaagggactaaaataggctccttaacaaaaagattcacagacaagaagtttgtttgtttgtttgtttgttttgaaatagtgtcttgctttgtggcccaggctggagtgcagtggttccatcttggctcactgcaacccccacatcactgactcaagagattcgcccatcttaacctcccaagtagctgggactacaggcacatcactatgccaggctaatttttgtattttggggggctacatgtgtttcagtatgtagcccacactgatctgtaactcctgtgctcagccttccgaagtgctgggattacaggtgtgagccactgtgcctgcccaagaacagttcattaatacatgcagcatatatcacacaggacaaacctaaatgaaaagtaacaacacagtggctcagaacactgccttacacagcagattccaaaagacacaataaatttgtagagaaataacaggaaaaagaaagttttaggcctccaaaggtgagaaactgtgcataggtaaatatctgagaggaagccgatgcagcaggatttctctgcggtgcctctggtaccgccgctggctgggcaaagttaagggttgtctccagtgaaggagagtttatattgtgcctttaggcagaaaggggagggaaacctgaacttttcctgtattttctgcttcttaattgccttaagctgaaaatcatttttatgtgaaagaggcataatctgggatgacgcctctgctttcctccacctgaagagaacctgtgtgctgctcctttgctttggacctctacctctgccacggagaaagcccaggccaacctgctggacaagcagggaccgtgagaaggagagttcaggtgtcccaatccaggccatcctagaccagccagcccctcacgagccccagctgatcagcacgcagccacttctgctatcttctactggccaaagtgagtccagggttcacccagattcagaggtggggaaactgagtccaccacttgagaggagtagctataaagacatacgagcgagaccagctgagcccagcactgctggccaagtcgaagactttaggaccagccacacatgttccgtggccacacgtggccagtggctccatattggacaatgccaatcagactcctcattctcattacatttgtatacccttctggccctgagatctttctatatccgcatctgactaagatgctctactagagaacagcatctacttcatatttccatcctttggaaacccaaagagccagcagaagttttgactttgcaattgatcctacacgttcaaatttctagcatctatcagaccgtgtaagatggaagagagacttacaagggctcccattacctagcccagggtatgtgctcagggctcttggcacttctcctcttggttacacatggttcagataatgttggccacttcttaacattagtttctcatggcttgattcctaggaagcattattcctcccattttaagagggcagccagttgagtgattcaatgagtcaggcccagtaccaggccccagggacacagtaagagacagagtacacatagcccttgctcttgtctggggtgcagacactaaacaaataatcaaacagattaaacatgcaattataggttttaattatgccctaagaaaaaacaaagccgggggcagtgacacaactgtaatcccagcacttcgggaggctgggacaggaggattgcttgaggacatgagttcaagaccagccaggcaaatgaatcttgtgggatagcactgtcaggctgggctctgagcctgcatcaaagcagaccctgtgtctacaaaacatttttaaaagttggtcaagcatggtggtacacggctgtagtcctagctacttgggaggctgaggcgggggtgttgcttgagcctagaagttcgaggctgcagtgagctacagtgagctgtgattcatcccactacactccaacctgtgcgacacagcaagacatcatctctaatttaaaaaaaaaaaaaaaaaaaaagaaggaaagaaagaaaaaaggatgatacaaaataagccgagcgtggtggcacatgcctgtaatcccagctactcgggaggctgaggcaggagaatcgcttgaacctgggaggcggaggttgccatgagctgagatcgcgccactgcactctagcctgggcaacaagagtgacactccatctcgaaaaaaaaaaaagaggcgggtgcagtggctcactcctgtaatcccaggactttggtaggccatggcaggtggatcacttgaggccaggagtttgaaaccaacctggctaacatggcaaaacctcagctctactaaaaataaaaaaaaattaaccaggcgtggtggtgcgcatctgtaatcccagctacttgggagactgaggcaggagaatcacatgaacctgggaggtggaggttgcagtgagccgagatcatgacattgcactctagcatgggtgacagagggagactgcatctcaaaaaaaaaaaaaaaaaaaaatgaagaagcggctgggcacggtggctcacacctgtaatcccagcactttgggaagccgaggcgggtggatcacgaggtcaggagttccagaccagcctggccaacatggtgaaacccccatctctactaaaaatacaaaaattagctgggcgtggtggcagacgcctgtaatcccagctacttgggaggctgaggcaggagaatcacatgaacctgggaggcagaggttgcagtgagtctagatggtgtcattgcactccagcctgggcaatagagtgagactctgtctcaaaaaaaaaaaaaagaagcaaatatgaagactatgtagtcttgaagaggacacaatcttttgaacaagacacaaaatccacaaactgtgaagaaagaattcactttcaaaatataataaataggacaaaactttttggaatttgcttgtggtgacgtggtgatctgatctcacaggtctatacatctaattttatactttacatataaatattttgtataaattatgtctcaatacttttttttacagaagcattgggggaaaataccataaagtaggttaaaaaacagatgacaagctagcagaaaccatttgccataaaaataagagcaaaaccaagaggaagcggaaagtcaacagaaaaatgagcaaagccctttaacaaagaaaaaagaaggccaggccgggcgcagtggctcacgcctgtaatcccagcactttgggaggctgaggtgggtggatcacctgaggtcaggagtttgagaccagcctggccaacatggcaaaaacccgtctctactaaaaatacaaaaattagccgggtgtagtggcacacgcctgtagccccagctacttcagaagctgaggcaggagaattgcttgaacccgggaggcagaggttgcaatgagccgatattgctccactgcactccagcctgggtgatagagcaagactccgtctcaaaaaataataataataataatgataataaaaaggagaaaaaggaaggctgttgattaataagcatgaaaagatgcttagctgcccagggaattaaagaaacgtaggttaaaacaacatcaagtaacatttttaacccatcagctatgcaaaagtgaaaatgatagatttagccatgtataggtggtggtagcacccaccaccacgcctggctaatttttatatttttagtagagacagggtttcaccacgttggtcaggctggtctcaaactcctaacctcaggtgatccacccgccttggcctcccaaagtgctgggattacaggcatgaagcactgcgcccggcctttttaattttttttggacagtcttgctctgttgcccaggctggagtgcaacggcacaatttcggctcactgcaacctccacctccctggttcaaacgattctcctgactcagcctcccaagcagctgggattacaggcacccaccaccatgtccaggtaatttttatgtttttattttatttatttatttttaattttattttttgagacagagtttcgctcttgttgcctaggctggagtgcaatggtacgatcttggctcatacaacctctgcctcccaggttcaagcgattttactgcctcagcctcttgagtagctgggattacagacattcgccaccatgcccagctaattttgtatttttagtagagaaggggtttctccacgttggttaggttggtcttgaagtcccaacctcaggtgatccgcccgcctcagcctcccaaagtgctgggattacaggtgtgagccactgtgcccagctccagggctggttatctttgtattttcaggtagccagttctccccacgccctctactgaaagatctcatgcatcacattttctccctttggggtaagaccagagaacctggcctgtcctgcaagtcttgtgttattctgatcccagtcttcatgaactccactcattcattcaacacacattatgggttgagcaccaaccaggcatcgggcttttggaggagctggagatagagcaggaccaagatagacaaaaagcaaaaacccctgccctgtaggaacttgcattctgatggtgggacacagccagtgaatgagaagtttgataaatggctaaattatttagcatattagaaagtgccaaagattaaagtggagagggatcaggggattaggaatgtatgtgtgtttaggggtgtgagttgttaatttttttttttaagacagagtttcactctgtcacccaggctggagtgtagtggcacgatcttggctcactgcgatgtctgcctcccgggttcaagggatgctcctgcctcagccccgctgagtagctggaaatacaggcgttcgccaccatgtctggctaatttttatatttttagtagagatggggtttcaccatgttggccagggtggtctcgaactcctgacttcaggcaatccgcctgcctcagcctcccagagttctgggattacaggcgtgagccattgtgcctggcttttttttttttttttttttttgagactgagtctggctctattgcccagactggagtgcagtggcgcgatttcggctgactgcaaactctgcctcccaggctcaagtgattctcctgcctcagcctcctgagtggctgggattacaggtgcctgccaccacctggctaatttttttgtatttcaagaccaggctggtctcaaactcctaacctcaagtgatccacctgccttggcctcccaaagtactgggattacaggagtgagccaccatgcctggccttttttttgtttttttttttttttttgagacagtgtctcactttttcacccaggctgaagtgcagtggagtgcaatggcacgatctcaggtcactgcaacctccacctcccgggttcaagtaattctcctgcctcagtctcccgagtagctgggattacaggtgcatgccaccaggctcagctaatttttttgtatatttagtagagacagggtttcaccatgttggccatggctggtctcgaactcctgacctcaaatgatccacccgcctcagcctcccaaagtgctgggattacaggcatgagccactacgcccagcagtttcttccacttctaatagactctgctagtctaggaaatgtaccaaaaagacagcatggttaaaggtcagtatttcatgaccttttttatacttcctatttttattttatttaggtttcttggttgatacaaaacgtcaattgtagtggtagggaacctgcataaggaagcttcctaaacagaggcttcaagagagaacactaataggccgggcatgttggctcatgcctgtaatcccagcacttcggaagcccaaggcgggtggatcacctgaggccaggagttcaaagctggcctggccaacatggtgaaacccccctctactaaaaatacaaaaattagctgggcgtggtggcaggtgcctgtagtcctggctactcgggaggctgaggcgggagaatcgcttgaacccgggaggtagaggttgcagtgagccgagaacgtgccactacactctagcctgggccagagagtgagactctgtctcaaagaaaaaaaaaaggtcgggggaggcttgagagaatgggttcactttcttcctccatgtgaggacccataggtagtgccagcagtgaggcacaggccctttccagatacgcaacctacaggcgcctatatcgtggacctcccagcctccagaaccatgagaaagtaaatttctgttctttataaattacccagtttacttaaaaaaaattttttttgagacagggtttccctctcttgcccaggctggagttaaggagcaccatctctgctactgcagtctccgcctcccaggctcaagcaatcatcctaccttagcatcctgagtaactgggattgcaggcatatgccaccacgtctggctaatttttctattttttgtagagatgggatcttgctatattgcccaggctagtctcgaactcctgggctccagtgatcctcctgccctggcctcctaaagtgctgcgattgcaggcttgagccaccacacctgaccttgcttattccttttcacaatttctctgcctacattttaacgggatctctgaagggaggtgagtcaaaagtgtgatccagttttgttgtattgaatagaaggtctttaaatttttattgtatttaaaactcggtagcagctgggcaaggtggctcatgcttgtaatctcagcactttgagaagctcaggcagggaggattgtttcaggcacaagttcgagatcagcttggggtaacacagtgagaccccattctttttttaatttcttttcttttcttttcttttatcagacagagttttgctctgttgcccaggctggagtgcaatggtgccaccttggccaactgcaacctccacctcccaggttcaagcgattctcctgcctcagcctcctgagtagctgggaactacaggcgcgcaccactacgcccagctaatttttgtatttttagtagagaggagatttcaccacgtaggccaggctagtctcgaactcctgacctcaagtgatacaccagcttcggcctcccaaagtgctgggattacaggtatgagccactgcgcccagccgagaccccattcttagacacacacacacacacacacacacacacacacacacacacacacacgagagagagacagtgagagagagaattagagaattagctggtcatggtggtgcatgcctgtaatcccagctactcaggacgctgaggaatgagaattgcttgaggcagaggctgcagtgagccaagatcgtgccactgcactccagcctgggcgacagagctagaccctgtctcaaaacaacaacaaaaaacaactactttgcactgtacagagactatatatccacaggccaacctccctcccattccggtcccacaccagccttttaataggttttttacaaggccgggcgcggtggctcacgcctgtaatcccagcactttgggaggccgaggcgggcggatcacaaggtcaggagatcgagaccatcctggctaacatggtgaaagcccgtctctactaaaaaaaaaaaaaaaaaaaaaaatacaaaaaattaaccgggcctggtggcaggcgccagtagtcccagctacttgggaggctgaggcaggagaacggcgtgaacccgggaggcggagcttacagtgagccgagatcgcaccactgcactccagcctgggcgacagagcaagactccgtctcaaaaaaaaaaataggctttttactatccggtttaaatagagaatttttttttgcttgttttttgttttctctctttttttttttttttttttttttgttttttgagacagagtctcactctgtcgcccaggctggagtgcaggggcccgatctcggctcactgcaacctcctcctcccggattcaagcgattctcctgcctcagcctcccgactagctgggatacaggcgcgcaccaccacgcccggctaatttttgtatttttggaagagacggggtgtcaccatgttggccaggatggtctcaaactcctgacctcaagtgatccgcccgcctcggcctccccccagagtgctgggagtacaggcgtgagccaccgcacccggcctgagaatgttcgtattctactcatgaagacttcgtaaatattggggtccagacctagatactacaacgttacaaatctgttctgatcactgctttgccagatccctggtaaatccacagctctggccgggggcgggggggccgcatgcaattcccttctttcctacagggggcgctgcagagaagggcagagcagagcgcgcagttcgcgggcaggggccgcttctccaggatagcgcgcgtccgagggggtgggtctgtgctagccctgcgcaacctcaggggcgggaacaactctggctctgcccccgccggctggagcgccttctcattggaggagggaacggtcacttggcagcgccgttgggattggaggaagagggtctcgggtggagtgacgctgaggcggcgagggggctggtggctggccgctgctgcccttcggtagctggtcccttaactcagtggtgaatggcgaccggatggagctctagggaagcgacagcagcggcgggtgggccgggttatgggcgccccgagtccgggcggcgtccccggtgtgccgctgacctgctgggtgggcgctgtcctcccggaggggggtccctttgctctcccggacccctttacccgtcacttcctcgccggtccctgaggcaggtccccggagccccgctgggcgtgaggtgcagggagcggccgcaggtggacccggggctggagggcgctcggccaccacccgagcgggtcttggccttgagcttccgagcgcctcaggttcagagctgcaccccacgagcccgggaggcggtggtccgcgccctgcctgggttgccccacggcgcccggcctccttcgaggggcctcggagcggcccggcccggcccggctgaggagtcagagctcgcgctccccttgcccgggagctgcagcccggctcctccgtgcgggcgctcgttcgctgatcgcgggcacctcgggccaaatccaccccctccgagacccgctccgctttctaggagtctctttcccagacctggtgccacctgttgctgggtctcccactaagccttcgagatctttggcacagcttcctttgaactcttcctccccgctgcggcttgagctgggcctgctaggagggttgctcagaattccaatgccagaaagaacgctccgtgccctgaaagctggagggaggagagaggacttcacctgggaaggaggagaaaagctttgaggggaggcacagttatttgtgtggtccttgaaaggcaagaagactggggcccgtggccctgaaaggggaggtggggctcccagcagaggggactgaggtcttcataaacaatgagggaccagagcagagggaaggaggacgagattccagactctagttccaaacaaggcacctgtgatttctcatcccctgggcagtgactgccaggcgcccttcagcggacacctgggacttgcagtgttatgtgcccagggattttcctggacgggattgtgaagtagcgggtaaggtcatcaggcaggttggggtcagatgcagatgttcgcttgtggtcattcctgaaatgagaatctggcagctcacgttatttcagaaggtgctaggatcacttttgacagtgataccaaaaaattgatggtattctggtgaccctgtgtgaaaaatgaaatgttcgtgcaatctggagacactactgggaccagattaatctgtttttgtttccttatttatttatttttgagacggagtttagctcttgttgcccaggctggagtacaatggcgcgatctcggctcactgcaacctccccctcctagattcaagcgattctcctgtcacaggctcccgagtacctgggattacaggcatgcaccaccacgcctggctaattttgtatttttagtagagacggggtttctccatgttggtcacgttggtcgtcaactcccaacctcaggtgatctgcctgcctcggcttcccaaagtgttgggattacaggtgtgagcctgcccgcccagccttttttttttgaggcggagtctcgctctgtctcccaggctgcagtgcagtagctcgatctcggctcactgcaagctccacctcctgggttcacaccattctcctgcctcagcctcccgagtagctgggattacaggcatgcacaccatgcccagctattttttttttttttttttagtagagacagggtttcaccatgttggctaggctggtctggaactcctgatctcaagtgacccacccacctcggcctttcaaagtgttgggattacaggcttgagccaccatgcctggcctatgccaattgtttcatagaatgtttacaatctagcttcctctgattttcttcattattagattcagattttttgttcagaatgccaccttcataggtgatgtacacttgtcagtggatcaccttaagagatgtcagtttatacctttgttgacaagtttgatcctttcgtgaagatggtgtctgtcagctctctccattgtaagggtacctgaaccctctcttaattaacagtcctatatggggtgatactttggaactgaatattctgttgactcttctggcttttagttcacttttatttcattaagaaaagattgagataagtggatttaggatgatagttttaatgtaccaaagtagaggatctgtatgcatgagaattgttttgatctcagaaattgttctgaaaattttaaactgcattgtgtacttaagtttaagattatttttatggatatataacttcaaggtgttggataccacggatcattcattcaatgtgtagacatttggccaagtgcaggctctgacctggaggtataagcagataggaggcgtggcccttgaccccaaatagatgagaatttggtggtaaatacataacctgtgtattagtccattttcatgctgctggtaaagacatccctaagactgggcaacttacaaaagaaagaggtttaattggacttacagttccatgcggctggggaagcctcacaatcttcatcgcggaaggcaaggaggagcaagtcctgtcttccgtggatggcagcaggcgaagagagaatgaggaaaatgcaaaatcagaaacccctgataaaaccatcagacctcttgagatgtactaccgcgagagcagtatgggggaggaaccgccgccatgattcaattatctcccaccaggtcccttgcacaacacatgggaattatgggaatagaattcaatatgagatttgtgtgcggacacagagccagaccatatcaacccgcatcccgtctttagctggaatgatggctgtattaggtacatgaggtctgatcttgtttgaggcaccaaagcctgctttcctgaagcatagttaatattttggccccacagaaaaggttatttgcagtttatgccaacttgttattgcaaaatcattgctctgaaatattcatttctaccaggaagctttggattgattttttttacccgcaatatctgaccaaccctccttattccagttgtttagatttcttttgtcatttttaatgaatctgaatgtcaaggaaggcagatctttttctggtatcaggattgattttattgtgtgataatacagatttcactaaactaaagtcctagaaggtcttgtgataattaaagtacaacccaataatatttttcttgtgtcattgctggaatcaagactgggtcagatttggaccactcatttattcactcaacaaacatttactgagtgtcaattgtgtgccccccttttttttctctaggtaccaggtcaagatgccatggtgaacaaaacaaagccctttcccacatggagctttaattccaatggagggagggagaaaacaaccaacctgtgggctggaacagctggtcgtgagtgtcagggtgagggcagaaagtggtagggcctgtgtagctggggtggagtgccatttgtcgttgggtgatggggagaccctcgatggtgatgatattggagtcaaaatccgaaggaagtgaggaatgagccaacagctcttggggtaagagcaagtgttccagtccagggagcagccgattcagaggccctgaaacaggagcttacagaagaaacattgagggggccagggtgcctggagggactatgtgggcaagggggagaggaggaggggagggcaggtcctggagggctctactggggggttttgagcagagatgagatgattctgacttttatttttatttatttatttatttttttgagatggagtcttactctgtcgcccaggctagagtgcagtggcgcgatctcggctcactgcaagttctgcctcccgggttcacaccattctcctgcctcagcctcctgagtagctgggactacaggcgcccaccaccacgcccggctaattttttgtatttttattagagatggggtttcacggtgttagccaggatggtctcaatctcctgacctcatgatccgccagtctcggcctcccaaagtcctgggattacaggtgtgagccactgcgcctggcctttttttttttttttttttgagatggagtctcactctgtcacccaggccggagtgcagtggcaggatctcagctcactgcagcctccgcctcctgggttccagcaattctctgcctcagcctcccgagtagctgggattataggcacccaccaccacacccgggtaatttttgtattattagtagagacagggtttcaccatcttggccaggctggtcttgaactcctgacctcatgatccacccaccttggcctcccaaagtgctgggattacagatgtgggccaccatgcccggccaatcctgactttttttaaagtacacagtccagtggtttttagtgtgttcagagagttgtataactgttgccacaatcaatttgagaatatttgcatcatcccgagaagaaatcccttacccagtggcattgacttactggctttgcgtggagaatagatggaaagcctgatcaggacaaagtctgctcaggctaggatccagagaccactgaggtaaggcaggccaggggcacacccacgaaccagggaggagcaggcaggcagtatccgagagaggtggttgactacagttggtagttcagtgggcatgtcctcttgttagagaggagtggaggggacagttggacctccaggcagggaggtggggtttggcaagagaggagtctcccattggacaatgggtagagttgcaggctgatgggcaatgattagtatcaaggcagctcagcacggagttgaaggacccataagactcttaatccccaggaggacccatgtggtctgcctagaaccccagcttcagaggaactggctgtgttgacccagtcattactggaacacagatgagagtggggccagctggaaggagggctgagtgctgagcctcatgctgcccacttggctcagtttctttgcattgctgccatttggggccagggtggtcttgaggccttggttgggagttaggtgactctgctgtggaggttagaggctagggagccagccattacagaccgcttgtgtttatgttctctatatctgttctctttcactgcaatatctctctgaaaatacatccttaaagaaataagctctttgagagcaggcatcttagactttttttcaccaatgttgccagcatatagaacgaactctgtaaatattcacagatggactgggcacagtggctcacgcctgtaatgccagcactttggagggttaggtgggcagatcacctgaggtcaggagttcgagaccagcctggccaacatggcaaaaccccatcgctactaaaaatgcaaaaaaattagcttggcgtagtggcttgtgcctgtaatcccagctactcaggaggctgagacacgagaatctcttgcacccaggaggcggaggttgcagtgagctgagatcgtgccactgcactccagcctgggtgacagagtgagactcataaataaataaataaatattcatggatgaaatgaaataaattcttaaatgtttgtataagtttgctattgttactgtaatcaattccccaaattgagctacttaaaactatattgattttatcatcttatagttcttacttctggtggtcaaaagtccaaattggatgtctctgggctaaaaccaaggttcaacgggactatgttccttctagaggctctaggaagaatttgcttccttgactttaccagcttctagaggccacttgcattcctaggcccatggtcccctcctcttctccaaatccagcagtgtaacatcttcaaatctttctgactcaacctcctgtctcctcacaaagaaacttgtgattactttagacccatcttgaaaatttgcagtaaccccctactgcaagatctttaatcacagctgattaaatatccttaatttaatcacaggttttttaaaattattttcttcattttgttaaatagtctagcaaaccatttcttcagcaccttttgccttgtaacctattcacaggttttggggatgagaattttaacaccttttggggcccattctaccacagtgcttagtctcatttccccttatattcttttatttttttattaatttgagacaaggtcttactctgttgcccagcctggagtacagtggtgctatcatggctcactgtagcctccccctcctgggcgcaagtgattctcacacctcagcctcctgagtatctgggaccacaggtgtgagccaccgtgcccagctagttttttattttttattttttgtagagacaggttctccctatgtggcccagtctggtcttgaactcctgagctcacatgatcctcccacctcggcctcccaaagtgctaggattataggcgtgagccaccactcccagccgctttttgtttttgttttcgttttttattttttaaagacatggggtctcgctctgttgtatagtgatttcgtgtcgtgtcatgcaatgatgtgatcatagctcactgtaaccttgagcttctgggctcacacaatcctcctgcctcagcctccccagtagctaggactacagagaagccaagacctcaggtacatgttcccacacatggccagtagctggagaccagccatggactgggcacagtggctcacgcctgtaatcccagcactttaggaggctgaggcgggcagatcacctgaggtcaggagtttgagaccagcctggccaacatggtgaaactccgtcttctactaaaaatacaaaaaattagccggccgtggtggcacatgcctgtaatcccagctactcaggaggctgagacaggagaatcgcttgaacccgggaggcggaggttgcagtgagccgagatggcgccactgcactacagcgtgggcaacaagagcgaaactctgtctcaaaaaaaaagaaaaaaaaaacagacattggctggccttgtggctcacacctgtaatgccagcactttgggaggccaaggtgggaggattgcttgaggccaggagttcaagggtgcagtgagctatgattgaacactgcattccagcctgggcaacggagagggaccatgtctctaaaacacacacacacacacacacacacacacacacacacacacaattgtttcctcagttctggaggctggaagtctgagataagggtgccagcgtggttgggttctgactgggagttgggtgtcttctgtgtcctcacatagcagagagagagagatcaggagattgaaatctcttcctcttgtaaggccacagtcctatcaatattacttggaatctgtggtcacacagttcagccttagtagttggttggcacacctgtgtcccagacccatttttctggaagtttccagcttcatgtgtttctggcagatcagggcgctgcctcctaggtcacttgccctgatggtatgggacagtcagtaagaagctaggctccggcagtgccttgaattactgcaaaaagcagtgctttatcacagcttcagcagaagccttccacagcaggagtccggggtgtggtctcacatcggtgggggcagacagcagccagtggtgactaatctgagttctttgttttctgagacggagtctggctctgttgcccaggctggagggcagtggtgttgtgatcttggcttactgcaacctccgcctcccggattcaaccaattctccttcctcagcctcccaagtggctgggattacaggagtgtgccactgtgcccagataatttttgttgttggcttttttttggtggggaaccagagtctcagtctgtcacccacgttccagtgcagtggcacaatctcagctcactgcaacctctgcctcccaggttccagtaattctcctgcctcagcctcccgagtaactgggattacagccatgcaccaccacacctagctaatttttattattattattaatagtagagatggggcttcaccatattggccaggctggtctcaaactcctgacctgaggtgatccacctgcctcggcctcccaaagtgctgggattacaggtgtgagccaccatactggtcttaatctgagttcttggaggcaggtttgcatctcatttgttcacttggaccagtgaattcctgatgccatcatggaactttgtgcagactagatgttcgggtggtcagagggtcagattgatttgggagtggcaggtgaggacctgggatagaagatggtctttctgctttggaagagaagcatggtgctgtgtgggggcttggggatgagctggcggtgggtggtggacagggggcctcagctcggaggccaggctgctcttgactgagccaggggccacagtagctgcccaggcaggagggatttgtgtgggcaccagagctctggcaggtgcacatttgttagggaggagcagtatccagatacaggcactgctccctctggagttgctgccagccagggagggtgcgtctctggcagcgaaaggcaggaatagctcctggcctaatggcagctggaaaagaagctgcaagaggatatgggtttgttgtggaagaggtgggggcttgaaaccctgaggaccaagaatcctgccctcaatttctgattgacactattgactaaaggtagcttttttcattggtggaggaagggcagtagttcatttgttcagcctcaatttatctatccatatcctgggcatgagagtagccaactctctcctgggatatatggagggatcacatgggacaatcaatagatataataattgtttcgaatcctgcatataccttataggtgtgaagtggttgtgtcctttgtgaattctcttggccaggcaccagtgggaggttagatagtgacctggagtttgtgttgaggaattagtagcctttgaatgcctgcctttgtagctgttgcacatacgttaggcttactttcaactaaatggaattagctaattgatttcttcagaggtccaagtgataacagagccaaaccttgctagcccataggggtgagagtctagcttagtgacaggttgtttttgggagacacaccctttgtgtcttcacttctctgtcccctgtctccgtcctgctctccctctcacccattctgccagctctgctggccccatgggcacctagagcacatgcacttggtgaatctagatctttcttccctgagacattgctgcaccccatcacctcatcacctcgcttccttcaggtctccaacaagtcaggtcatcctgaatattttctgtaaaactttctgttaaattctaccttcattattctttcattttagcctgccttgtttttctctgtggcactcatcactatttgatattgttgcatatttatgaatctattttctgtcttcttggtcgggaacataagctccattaggatggagactttgtctgttttgtttagattatattctcatcatctgaaatagtgcctgtagggcctcaaaaagttcatagcagattaatgagtgaatttaacaagtagacactattgatggtgctgagccacatctgggccctctctcagcgtggagcggaattatgcacacaggcgagtatacagactttgatgcctggtgctgccgttttgcaaaaagacttctccaacccactgtcttctttttgaatttttaaataaatctggccgggtgtcgtggctcacacctgtaatcccagcactttgggaggccaaggcgggcggatcacgaggtcaggagttcgagaccagcctggccaacatggtgaaaccccatctctactaaatatacaaaaaaattagctgggcgaagtggcaggtgcctgtaatcccagctactcaggagactgaggcaggagaattgcttgaacccaggaggcggaggttgcagtgagctgagaccgtgccaccgcactccagcctgggcaacagagtgagactccggctcaaaaataaataaataagtaagtaagtctggatattttagtcctggaaaggataccacatttgaagtcaaagacctgatctgaaattctgtcttcaccattacttgctgtacctctgataaatgccctaacctccctcacccatgattttctcatctatgtaatgggaaaatacttgttctgcctacctggggcttaacatttattgtaaagatcaaatgaaatgagtttctccccacgccccacagtgtattggagtctggcagttgtctcctggccactgtcctctgttgcaagttgagaaatgtgatctttacttcctgtccaaccctaaagatccggaaggcgtggatccgtcacagagagttacacaacattgtctacccagatcccaattgaagaaaatccacaggaagacaaggcaggctcttttcatcctacccataatgactaatccatcattgcctcagtaacttgatcgtcagcccgggtagcagatatccctccccgaagctgatgaaacagggcagagaggaagtatccaagagaaaagggagggaggtttctctaaaacaagacctacaataaggacattattgtctaaaggaaataaataattgaaagtttctgaaaactgagatttcttcatctcaaatgactttattagcccaaaaaagtacaaaaattattttcctcacataatattaatggtgggaatcaaaatgaatcagtagatgggggatccagattggaatgctgctacaccatagtagaatccttctgggaacctacggtgggaaaggatcacttgtagcaaaaagatccagagttgtgcagagggaccacgtcagcagtgatcacttttcagtaaccgtctttttctgctgatcattttagggtcactatgaggtcagactggatggtgggagtggataccaattttttttttactgcctacatatgcttcatgagctataatttgtctttttttccaccacagatctctgtttcttttccttactttcatgcccataatttatcttacagatgtggtttaatgctttgctgatttgacatagctttcttttccatttcattgattgctacttaaagatgcacaaggctttcagggcctcacatgaatgatgagatgagatccaacatcaagaatctgaagcaaagacacttgtggtttcagaacgtgtaaatagagcttggtgattttctgttgagtttgttgggggaatgcataatgattaggattaggtgtttttctattttaaaaagttgcttctagaggcatagaagacaattctgatacactatttaaattttccatacaaatccaggcatgtttatgcaaaagaaaaaaaaacaccagtacaaaacaaacaagatatgatctgactgggcatggtggctcacccctgtaatcttagcactttgggaggccaaggcaggaggatcccttaaccacaggagttcaagaccagcctaggcaacatagggagaacctctttaaaaaaaaaatttttttttaatttaaaaaaaaaaagatcaaatgagacaaatgctattcacatgtttcttgtcttgttatcgccacttcaaattcccatttattttgtccagtgaacatttgaaagactcagatatgaagttcaggggccatgcactttgtgcctctttccccagtattaatgcacttacacctgtctatttttctctttcttttagtgggggagataatgcctctttcttttgtgtacagcaggacagtgggtcattgtgtatgtacagaagtgatgtggattcctcccagactcattagtgaccagggctgctgggcctgtttgggtttcctagactagagaagaagacaggaaccaggatgggaagacggacatgttacattttaagctggagcttcccctgcagtccacggagcacgtctcggtgtgcagctcatcctgactttctcctatcgattacacgtgagtcagtccgctgggaggctgtcctctcccttgtgtctttttagtagaccagagtccttcttctgatttctagaagacccgcactctagcgggccctcttccccatgtagtttgggatttaacttaggccaatggaatggttttcactagtcatattaactgacatgggctattaatagctcagaaggttctaggcagtgtttcctgcatggctacgctaggtatgtgagggcctcttaggtttcagccaagacaggtgactcttagcacccagagcccttcagatctgatggcggttggtattgttggtccttctaatgatttcagaggatggcgaccctcgtgatgcagagcatggcgtttctccagtcctcctttcctgtcccgggatcccagttatacgtgaacggagacctgaggctgcagcagaagcagccgctgagctgtggtggcctagatgcccgatacaatgtaagggcgcttctattgtccagctcctttgtttctgtgtgttactgttcgtcgagttctttgaagagggggataaaaagtagaaatatttctctgtgggtggctttcaagggaccttctgaggaattcttgccatgaagtcttttcagactatgaagatacacctgtatttacacctagagggagagagagaaaaaatctgtgtttgttgaataactaaattgtttattgcttttaggtacacatgtgcacgtgcacacacacgcacatacattctctcttccatacacatacttggagtatgtgtttccaagagatcatgtaaaatgttcatgtaaagtatttcagaaatactactagacagtttactgaacccataacatatgccagccttgagtgcaggggtttatacgtatcatctcctttaatcatcacagcaacctctgagctgcctgcactgtcctttcccagcactccagccagtggtcccattgctgtcccccttgctactttgcagcccccctggcctttttgctgttccttgaacccaagaagcagctgccacctcagggcttttgctcttgccatttcctctgccaggcatccatgtggcttatctaaccatgccattcaggtccctgttcagatatcgccaagcaccatccttccctgacctgcttctagagtagcagcccctccctccaccatctctcagccagactctccctcctgccccctggcctactcaggatatgtgtttgtgtgtttatttgttgtctccctcagccttgacagcagtggcatctcatacactatcatgtccatgcctagcacactgcctatcacttggtaagctcacagagaagcttgtttgaagaatgaactcattttggagaagacaaaactggggcttacagtgattaattttcccaaaggctcaaatcttgaaagtataacagagccaggaactttctgtgtccagaactcatgcatctaatcactcattcctcacctttattttatcacagtggggaccatagaagatgagctgctgtactttggaaaatgaaaaagggaaattatcagttataaggcatcctcccacctcagcctccaagggtactgggaatacagacatgagccactgcacctaaccttttttaaaacaatttcatttattattttatttattttttgtgacagagtttcactttgtcacccaggctgcagtgcagtggcgcaatcatggctcactgcagccttgacctccccaggctcagatgatcctcctacctcagccttctgagtagctagctgggactacaggtgcatgctaccacgcctggctaatttttacattttttgtagagacggggtttgccatgtttctcagggtggtgtcaaactcctggacccaagtgatccactcaccttggcctcccaacatgctgggattacaggtgtgagccactgtgcctgcccccgcaccccctttttaataaaaaaactagactctcactctggcccaggctggagtgcagtggcctacctatgactcattgtgcctcatactcctgggtacaagcagttcacccacctcaccctccctggaagctaggactggaggcaccacaatgcctagtttattgggatttttttttttttttttttttggtagagatgggatctttatgttgcccaggctggtcttgaacttctgtcctctcaagcagtcctcctgcctcagcctcccagattgctgggattacaggcatgagccatgacatccagctgagagagattttataaatagctttaaaatctatgaaacatggacactgaccacttattttcatccccacagaaggaaggacagagtaaatgaacttaaattgttgcaagagagtttattcatctttgaagaaaagattattgatactgcagtaggccaccaagaacaagagggtgctctgtgggaccagcctgggtgggcagaagggtagaaggaaaaggggaggagtctcccaggtgctcacaccacatcctcctcccgtgtcccaagtatctgtgaccaacgggaccagcccctttgcctatgactacgacctcacccatattgttgctgcctaccaggagaggaacggtgggtcacaggtagagcccattcagccgctgctcaggactttcaaggttagtgggggcaacagagacaagcagaactggaacccttgtgatgaaaatgtcaaaacccgtgaatgctcaacgatgggagcaaatgtgctgcccaggatttgtctttttcgtgctgatcttcagacccgggaccacccgcacccctccagcatcccgtggtccagcaggctagtgatggttaaatgccatcattatcccaccactggcttggtgggtttctcttttaaaaaatataagcctaattgagttgttccccatagcacacctataacagatgccaaagacaaattgctgttcatgcttttcagagaaaaacagagaacgtgggtgagtctccccacattagcagtccagcaggagaacaggagtgcccctgtttcctgccagaaggcacagtgtgctcattccttacagagctctaggccatggcatgtgttcactcggcatgtttggctggtccgtggctgaggttgtctgtgaaggcatctagagtgttacttggcaaccagacatcatggagactatacacaaacatttcacaagggggaacgactgcaaatccagtattgctttctgattttatgtcatacattagcagcatttttataatagtaaagggaattctaaaacaaaactaaaaagcacggcccaagaaatcgatgttttttctttacttttttcactctttattcttatgtggattttaaaaatatacctattatgatagtgttgaaacactttggcatccaaattatttgtatttccatgtaataaaagtatttccccacactgctacaaagttctaattttttttttttttgaagagacacagggtcttgctctgatgatcaggctggagtgcagtgctgtaatcatagctgattgtaaccttgaactcctgtgggctccgtgatcctctcacctcagcctcctgggccgctaggactacaggcacgtgccaccatgcccagctaatttttttttattttttagtagagagagggtctcaatatgttagccaagctgctctcaaactcctggcctcaagtgaccctcccaccttggcctcccagatccttgggattacaagaatgcgcgactttgtccagacaaacatattatttagagtggcaacatgttattctttcagttagccattcagcaattgtagatgatcacatgaccatttccaagtttttgacgtattttaatataaatatcggattagtcctttctcacacagctgtaaagacatacctgagactgggtaatttataaagaaaagaggtttaactgacttagagttctgcatggctgggtaggcctcacaatcatggcagaaggcacctcttcacagggcggcagaagagagaatgagagcagagctaagtaagcagatctgataaaaccatcagatctggtgagaactcactatcatgagaatggcatggggaaactgcccccatgattcaattatctccacctggtcctgcccttgatccgtggggattgttacaattcaaggtgagatttgggtcatagagccaaaccatatcaaatgcttttttgacaagtatcttaacaaattatttgttaatctactttgtacatgcaatttttaatgctttcgaatgatttcttttagttctgaagtacagaatatcccacaggaataaaataatacttttatagccgagcgtggtggctcatgcctataatcccagcactttgggaggctgaggcaagcagatcacctgaggtcgggagttcaagaccagcctgaccaacatggagaaaccccatctctactaaaaatacaaatttagctgggcatggtggcacctgcctgtaatcccagctacttaggaagctgaggcaggacaattgcttgaacccgggaggcagaggttgcagtgagccaagatcactccattgcactccagcctgggcaacaagagcgaaactccatcccaaaaacaataataatatttttatagccctaatatattttgccaataggctgccccatcatggattaatggttaatgattgtattgcagttgccaccatcctgaatgaccccaacttcatctggctggtaggcagagccatgaaactccatttgtgattaatgttgtcatccagtaccctgtggaaaccatatcatatcctttctgttacagaggcagtggtaactcaacttccaaaaaggaaagattccaaagttgtccccagcagaaacacatgtgggagcctttgattggaaattgctacagcactgtatctccacataaatgactatgtctgctctttatacaggggggaggatcatttcctgttcgtatttctttctgagccattcttctaagtcagaatcatccatatttattttttaataatcttttattattcctttcatcttaagagtatttgaaatattcttgccatatcctgtctggcctaaaccttagtttactttgacttataccatgtggtccccaactctttaactgtctgtccttatcagccaggattctgggcgatggtaaagttcgcctgggtgcagtatgtcagcatcctgcttatcttcctctgggtgtttgaaagaatcaagatcttcgtgtttcagaatcaggtggtgaccaccatccctgtgacagtgatgccccagggagaagtgtgtaaggagcacttatcctagaaaggccgtttctgaagactcagcaggaccatggctgcctcattgtcatcttctgggaacgtcttaggaccttttgaaagagcccagcggacacctgcgggcttgtgtgcttttccctcagagacaacggttctttccagttttgctctacacagttccgtatcttcagagctcctgcagaattgtcagggactagtttgtggaaaggtctgagagttcctggaggctataattagctttttgggtttttcttctttgccttagcgttgaatttcaggagaaaattgcagtcagttcagacatcttggaaagagtcccatctctggtcaagcagagacttttcctctgttgaactgaggaacacactgtgcatttcttccttctgttgtgagccactcttactcttttcagggctctcttgtgacaaacatgccaatcactagcactttgcacccctgggcttctccatttcccattcacagctttgatttccagagctgaggcctttaactggagacctggaggggcagggcccaagggcaagggccgcattagcacaggcaatcagggagggccgctgaaggacacttggaccgtccacctgccccagcccaacagtcagtcatctgtcatcagctcagctgagcagccctggatccctgcccgactgtggctggctctttgcccggtttttccctctgtctgtgcccctggatggcaggctgaagtcagaggggctgtttcattctcagccccctcagcagcactgggggaagaaagcattgtcacaacaggttctttctggccctcacccaacagcctgggcacttggccctcctcctccttgacagccctcccccttcctgcaaaggacaggggcgacaggggttggtgttgggattggctcccgctgcctgacaaccacaagtttatttggaagggctagcgggaagcccagcggctggcgtttcccttgactaaggaaaagggtgcccatcagagtggggcgggcagctttgggaaggacacaagaagcagtgagggtgtaaagaggatgctggcctgggcaggccagtccagcctggccactagcagaataccaagcagtccagtggattatcctcgtggctaagcaagtgtctgcaggagcagagatggctggaaggggcctctgcacacggaagatggcttgttcagcccattcacctcctgaggatgcgtgcagtctcctccaagaacacatggagctgcttcctgatcccaagcaggtcgttgcccctggaaggacatggctccggtgatccatgcttcatgcccacccagaaacacacccctcagtgtgtgcctcagtttaccttggagatcatttttcatctccagcatccgtttcctttaggctgactaaaaacagttttggaaacaaagctattttgaagtattcaagcagaggaattctctaacactgtcccccttgtctttttttaatattctggctattttagatgcctaaatttttttcttgagatttatttatttttatagatggggttttcactatgttgcccaggctggtcttgaactcccaacctcaggaaatcctcctgtctcagccccccaaagtgccaggaatataggcatgggccagtgtgccccgtctcttgagactgaaatgaaaaattgcttgtggtttaaatccccagaatttaatgaaacatgggaagatggctaaagatacgtataaactttggtttgcattttgttaaattatttgaatgcaaaacttgtataaagaatccattatgttctgtagctttctaattaaaatgttcaacatggaaggttgtattagctatttttgaagcaaggctttggagggaggaagaatagggagggctgaagttgagcacacttctccgagttactcaccagtagccttgctttgccacggctctgcccaaagaccttggagcttttgttagctctttggcaaaagtttcctggtccaaccccaagtatattagtttttagtggaatccacatggcatatgggagctgcgggttggaaaattacaactgtgtctggtgaatatcaaattatttacggatcatttattggtagtttctgaaactgaaccacacagcatagttgcaggaggaagaaaggtaagaaatatacaggattttatcaactgagctgaagccataggtaagtcatatttgagataattattgaatttgttttgtgggagagaaatctctaattcttccaaatgattagtccagtttctgccaaagcaggtgaggtgtctaatgccccactgagtgcccatcttgaagcttattaaagcaattctctctctttgcgtccctcttaccttatcctccacatccttgccccctctccttattcatgctttgtaaagaggttttggagctttctggccagataacttctaaatactgattaaggaaatgtaaattacatgatcctaaggaaaataatgattgccggccaatcccctacccagagttatggttggccttctggcacacatctttctatcccatacactatttctaactttttaaaaatcatgaacaggccgggcacggtggctcatgcctgtaatccgagcactttgggagacagaggcgggcagatcacatctgagctcaggagttcgagaccagcctggacaacatggtgaaaccctgtctctactaaaagtacaaaaattagcctggcgtggtggtgggcacctgtaatcccagctattcaggaggctgaggcaagagaatcacttgaactcgagaggcggaggttgcagtgagcctagatcctgccattgcactccagcctgggctacaaaagtgagactccatctgaaaaaaaaatcagtatcaaaatcatgaacaacacttatatgaaccctgtacataaatccttgtttctggtttatttccttaagatagctcctagaaaggaattaccaatccgaaaagcttacgaccattgtcaagaatcttaattcatcttgccaaaccacaaccaggaaggactgcttatttcttctcccagcagcagggagttcttgattaagatcccatcactaatttgacaagacctagaaatgcttcatccttttcaggtgcctgggctctgccctgttatttgtaaaggggatttttttttttttttttttttgaaacagtttcgctctgtcgcccaggctggagtgcagtggtgcaatctcagcttactgcaacctccgcctcctgggctcaagcagttctcctgccttagcctcctgagtagctaggattacaggtgcctgccaccacgcccggctaatttttgatttttagtagagacggggtttcaccctgttggccaggctggtctcgaactcctgacctcgtgatccacccactacggccccccaaagtgctgggattacaggcgtgagacaccgcgcctggccggattttttttttttttttttgcgagggagtctcgctttgttgcccaggctggagtgcagtggtgcgatctcggctcactgcaagctccgcctcccgggttcacgccattctcctgcctcagcctcccgagtagctgggactacaggtgcccgccaccaggcctggctaatttttttgtatttttagtagagacggggtttcactgtgttagccaggatgatttcgatctcctgacctcgtgatccgcccgcctcggcctcccaaagtgctgggattacaggcgtgagccaccgcgcccagccctggccggaaatatcttatttattgaaacgtatcatgtctttatctggacctgctatgaacctgcgcctctgggcctaggctagggcagggaatgcgcaggaggaggggaaaagctgcggacccagctcctaaggccacctggtctctccgctcttcccagttgggacactgcaccgggtccatgtgtcatccaggtgccgtggccttgggaagggtttggattccaagtcacctcgccgcaagatgcccccgagagtgaggagggtcaagattaagattcgacttccttcactagggcctttgtcctcccaggagccccccttctctggggatttggggtggggtggggtacaacgtttctctgtagtcagatgagaggtgactctgccagcgccctttgacattctgggctaaaagttgagcctctcagagccccagcccacctgggccctcctgccccgccctcggccggcgcccctccctcgaggcgtggcccctcggtcgggggtgggccaaccggctccttccttcccccacggcgctagctcccgctggccacctcgggaccgcagccacgtctgaaagcgcctcattgtgtgcgctcgggcgggctgcaccgggcagcgccgagggttgccggccggcgcgcggggagtagagggcgcgggccgcagtgccgggttccagagggagctctgcgccgggtccttccctgtggtagccccaggacacccccagcctcaacatcccattctgggactcctgccctgttcccacattcgttctacctcgagtctccaggagcttccagtggcttggtcaccgccaactctcgtccatgcctcttagagcccctttcccggcctcaccgggtgtcgcttaatagtcttgggaccttaaggagcaagtcagcccctgcggaccctcccagtgaagagaaagagctggctgtgcggtggaatttggaagagacgacgtttgggagcctttgctgagtccagggagagaggcgtcccccaccgtgccgctgcagctcgggcagagccgccaagctttggggtacgttggttcttcattctccgcgggggatgtccccacactcgggtcggctgggggtcgggctggtggcacagctggggacgctcctctgtcctgcaccggggacctggggtggcgggaagagctgggagtggcttttccatcagcaacgtggaaagggcatcgccgctgttggcgccggtttggaagtgtcttgctggggcttcggctgcgcaggagaatcctcactgcgaggggagaaccccttttgtctcgatacttgagtttaaaggacatgactttcaaaagctctccaagggctttttccatagaattattgagagacagtagcttggcgggttgatttggaaccagacccaggggaattggagtcctgcgtcactgcttctgacggcttcatcttggcttcaggacaggccctctgggcctcaggacaggtgcctagcaaatgttgactttcctttctcctccctttttcacagaggcaaggcagtcaagtttcccatttttgaggatgggaaaactgaggcctgaagagcagaaaggagttggctgacttaaacccacaagcgagtaggccaagtaccagggcctctgagggtgctatgctgctccgggagctggggctgggctcctctccagcctgagaggccggaacttttctggctttgttctacaaacagagacaactggagtatagagagccagagagtgacttgctctaagtcacacccctcactggtagtagagacaggatttgaacccaatccggcttcagaagccaagcttctaaggccagtcgcagtggttcacgcctgtaatcccagcactttgggaggccgaggcaggtggatcacctgaggtcaggagttcaagaccagcctggccaacatggtgaaaccccatctctactaaaaatacaaaaataagctgggcatggtggtgagtgcctgtaatcccagctacttgggaggctgaggcagaagaatcacttgaacccaggaggcggaggttgcagtgagctgagattgcggtacttcactccagtctggacaacagcgagactccatctcagaaaaaacacaagccaggcctctagccatgactttccagcgtcttctgttttgtttgcccttgtggggaccctgtctgtgcctgccacattctgttgctggggcactggggcacctgaatctggtagagccattgtccttgggttttcctcattcaaagactttcccttggattcatagaatataagtgtggcccagaaagaagattgctaagcaaatactcataagtgcactatgtgccagactttgttctgctttacaaatatccactcactcactctttcaatcaagcccatgagttgggttatattgtttcccccactttacagatttttttttttttttttgatacggagtctcgctctgtcaccgaggctggagtgcagtgaaagtgacataatctcggctcactgcaacctctccgcatcccaggttcaagcgattctcctgcctcaaattcctgagtagctgggattacaggcacccgccaccacgcccggctaatttttgtatttttagtaaagatggggtttcactatgttggtcaggctggtctcaaactcctgacctcgtgatccgccctcctcagcctcgcaaagtgctgggattacaggtgtgagccaccgcacctgaccttccagatttttttttaaccaagcaggcacagaaggaagtaacttgcacaagatcacgcagtaaatggtcaagtgggattcacactcttaacctcagtgctctatacctactgtctttaatgagacagacaccattctgcttctagtgtcttgggcggggaaatgggtccagaaatacatgaacaagatttttctgggtagtgataatagtattaataataatttctcactatcacctattagtcaatattgaaatatatctgattacctctagggtatctcctggcagatgtttttttcaaatcagaatccacacaaggtccacatgttgcatttgactatgtgttttttggttttggtttttgtttttgtttttgtttgagatggagtctctctctgtcgcccagactggagtgcagtggcgtgatctcagctcactgcagcttcggactcctgggctccagtgattctcctgcctcagcctcctgggtagctaggattacaagcacgtgccaccacacctggctactttttgtgtatttagtaaagacagggtttcaccatgtttggccaggctggtctccaactcctgacctaaggtgatctgcctgtcttggcctcccagagcgctgggattacaggcgtgagccaccgcgcccggccctgagttattttcctataaaatagtctcttcctttttctgtcatagatttggaagtatgatttacttttaaaaaataaggtaattaaaataatggttaataaaccagggaacattccagattatctctgtaatgagagggctctgtcaatacttagggaaacaggcaaatgtgctaaaagcacagtcccactccgtaccctgtgtgtgttgctgcagagaagaagtggggaaccctcagactttatggcctccagctgtaaggtactacaatttagaccaaggcaggtgctgtgtgaagtgccctgtgagaatccctaaagctcccagccaaggaaaagggtttgtgtgttgctatctgcttatctgctctgaaatgccccagggcagtggctctgagccagggaggcttgaggctcctttcccaggctaattagaattcatttcccagtagattttgaaggcagatctgttttctccatcagcatctgggccttgcagtcccagccccctgcctctgggggatgccatgcagctgcatcaccaggactggcagagttggcagatgtggccagggcttgggggccaggcacagcctcagtcacagcctggcccagcccacagtcctggggttgagagtgtgcatgggagctctgacaccttttagtgcagagcgggtaattcgtgcccctggacttctctggctaactgggtactcaggtcagctgggagatggttttgcaggcacctgtctctctgcagtgatagctggacaaagacaaactccacctaagcctcctttaaccaactagaaatttctgattttatcattgtagtaaaagtctgatgtagactatctggaaaatgcagcaaagcactaataagaaaataaattaccagccgggcacggtggctcacgcctgtaatcccagcactttgggagactgaggcaggcggatcacgaggtcaggagatcgagaccatcctggctaacacggtgaaacccggtctctactaaaaaatacaaaaaattagccaggcgtggcgtcgggcacctgtagtcccagctactcaggaggctgaggcaggagaatggcatgaacctgggaggcagagtttgcagtaagccgagatcgtgccactgcactccagcctaggcgacagagcaagactccgtctcaaaaaataaaaataaaaaaaaattacctgtaaactggccatagaaaaataaacaccaaacttcagtgtgcatctcccatactttgtatttttttaaataatggctttattgagatagaattcatgtatgttaaagtttatatatattatatataagtatattatattttatatatatatatatatatatatatatattttttttttttttaagacggagtttcactcttgtcacccaagctggagtgcagtggcttgatttcagctcactgcaaactctgccttcctgacctcaggtgatccacccatttaatccaaagtgctgggattacatgcgtgagccactgcgtccggcccaaagtttatattttaaaagtgtacagttcactggcttatagtatattcagtgttttcacattgaactttgtatatatcagtactttattcttttttattgcagataattgtctactgtgtggttacaccacattttgtttatctattcatcagctgatagacattagggatgtctccactttttggctattacgaataatgctgctgtgcaaattcatgagcaggttttcatgtgggcttgtatgttcagttctctagggtgtgtacccaggagtggatctgctgtatcatatggtcactctattcaacctttcgagaaaccaccaaattgtttcttcaggaaatgcaccatctgacatccccattttatgaggatccccacgtctctgtcatctcaccaacacttgtaattatatattatttattataattacctttttaagattgtaaccttcttggtggatggaaagtgacttgtcactgtggtttaatttgcatttccccgctgcctaaaaatgttaagcatctttcctttttttttgagacggagtttcactcttgttgcccaggctggagtgcaatggtgcgatctcggctcaccacaacctctgcctcccaggttcaagtgattctcctgcctcagcctcccgagtagctgggtttacaggcatgcgccaccatgcctggctaattttgtattcttagtagagacggaatttctccatgttggtcaggctggtctcgaactcccgacctcaggtgatccaccagccttggcctcccaaagtgctgggattacaggcgtgagccactgtgcccggccttttttttttttttttttttttttgaggcaaagtctcgctcttgttccccaggctagaatgcaatggcacaaccttggctcactgcaacctctgcctcccgggttcaagcgattctccctccccgccgagtagctgggattacaggtgcctgccaccacgcctggctagtttttggtatttttagtagagacggggtttcaccatgttggccaggctggtctcgaactcctgacctcaggtgatccacacacctcggcctcccaaagtgctgggattacaggcatgggccactgcgcctggttgagcatctttccatttgtgtatcttcttcagagaaacttctcccaaatcctttactcattttaatttttttctttattcattcttaaatttatttattcttatatttcttaaatatgcttattcttaaaaaaactaaataggatatttgcctattttgtgttgagttgtaagagctcttcttatattctgggtacaagtcccattctgtagacatgatttgcaactgttttctcccatctgtgagtgttctttgatattcgtgtatttttaacacccagccaactgtgtgaaaaatacatgcacatctaagaacacacacagatgggagaaatgtatatatgtatgtcagctttgttacttctgttttccacttgacactatgtagcgaagagctccaaggttacactgctagacttccaactctgactcagcagccactgtgtgaattcttcccgcaagtcccttttcttttttctttttttttttttttttttttgagacagagtcttgctttgttgcccaggctggagtgcagtggcacaatctcggctcactgcaacctccgcctcctgggctcaagcgattcttctgcctcatcttcacgagtagctgggattacaggcatgcaccaccacgcctagctaatttttgtatttttagtacagttggggtttcgtcatgttgtccaggctggtcttgaactcctgacctcaggtaatccgcccgcctcggcctcccaaagtgctgtgattacaggcatcagtcaccatgccctgcctcccttaatttttctacacctcactttcctcatctgtaaaatagggatgatcatcaaggtccaatgtcatagggttgctgtgaggatcaaatcagaaaatggatgggaaagactgggcatgtaggaaaccctcatagatgatgtttgcaggggttccttgttcctgccccccatgaacactcaccttccatctttatgttttatgttttgtttttggttttgtttttctttgagacgaagtttcactcttgttgcccaggctggagtgcaatggcgtgatctaggctcactgcaacctccacctcccgggttcaagcgattctcctgcctcagcctcccattacagacctcatgtgatccacccaccttggcctcccaaagtgctgggattacaggcatgagccaccgcacctggcctgttttatgttttatgttccctgctttatgttttgtctcattttctgttttcttctctcttctttcttcctctttcttttgctccttgttagccttcccccttccaaatgcccagggctccactagagcagtttggccccagttgtagctcttgatgcctcaaggccactgactcctcattgggtatgttttttccagcctttgggattcttttttttttttttttctccttgtgacggggctttgctcttgttgcccaggctggaatgcaatggctcatgatctctgctcactgcaacctccacctcccagactcaaacaattctcctgcctcagcctctggagtagctggggttacaaataggcacctgccaccatggtggctaatttttgtatttttagtagagacgaggtttcaccatgttggccaggctggtctcaaactcctgacctcaggtgatccacctgcctcggcctcccaaagtgctgggattatagacatgagccaccgtgcccacgtggctttgggattctcgaacagggatgacctccagccaactctaaacagagaaatctaaacctgggatctatacctagtgttacattttgttcccttagattctatgaacagggctggaacaggctggtaacaagaagagctgctgtgtcttgttgttgttttggttttgtttgtttgtttgttttttgaggtggagtgttactctgttgccaggctagagtgcagtggcatgatctgagctcactgcaacctctgcctcctgggttccagcgattctcctgactccgcctcccgagtagctgggattacaggtgcccaccaccacacctggctaatttttgtattttttctctttttttttttttttttttttttaggggggacggagtctcactctgtctgtcacccaggctggagtgcagtggcacgatctcagctcactgcaagctctgcctcccagattcacgccattctcccacctcagcctcctgagtagctgggactacaggtgcccgccaccacgcccggctaattttttgtatttttagtagagacagggtttcactgtgttagccaagaagatgtcgatctcttgaccttgtgatccacctactcagcctccaagtgctatgattacaggcgtgagccaccacgcctggccaaatttttgtatttttagtagagatggggtttcaccgtgttggtcaggctgtcccaaactcctgacctcaggtgagccacctgcctcagcctcccaaaatgctggtattacaggtgtgagccaccactcccggcctggagctgctgtgtcgagggtaggcagagcttggctggaagtgagtgctgcccagagagccaggactcctgggctccagctcaaccctgctatggaaaaccatccttggtcaatgtcttgcttttcctatttgcaaatcaggcagaaccatagctccttcctaggtctctcaggccaggccaagaaatggtaagggatgtttagaagatcctgtgactttaaggtgctcggcagaaagccatgtgggctactggggaattcctccctctggatgtggaccacagagtatctgagtgcaatggcaagtatttgttcatgtcctctactcatttgttgtttgggacattccttgacatgactcttactatcttcttcagtcaggattctttactttttattgaagtatgccattcttcagagtacatataatgaaagtacagcttggtgaactttcacaaactgaacacacccatgtaattagcactgtcagagagaaacaacgcagggccagcccccagaagctctgcccctgcacccttcccacctcaccccaccaagggtttcactgtcactatctaatcaccactcttgggtgtaagtgacagaaactcatctcccaaggacttaagcagaaagtgaatgcttccctcagctgaggggtccatgggtaagccttgcacaactggatccaaatgcttagatggtgctgtcagcttgcctgtgaactttctctgctgagttctcttctgttgttgattttaaacctaggcagtttcctcttcccttgtgatcaacaaaactccagaattgcagttgagcagatgtcccattcctaggccaatagttctgtgtgggctgggggggcagggtcagtgtgtggaatatagaggtgggtctgtcttgggacacatgacccttaggagccgggagtggtggatcagccccatctgagccacatgcacttttgagtgggtgagggtgattccccagggaaaagcctagagactgttatcagaaggaaaaacgggtactgagtggacagaaccaagcatgtccagtgcacctgtgcagtgctgatcctcacactagtcaccggagaacaggtgttcgcttgcgaaaaaccacaatccaaagttccgggttgctcacaggcaagaagggaaagcaaagtacaatgcagggcagagtggggcatgccttgacaggcacagaggaggtggctgcaggcgaggctgaaatgcctcccagtgggagtcagggctgcgcactcatagcatgggtgagggagttgggagagatggagcttgtttgatgaacactgattattcccggccatctgaagcctgggatatgggagataaggttggaaagctgaaggttactgaaaaggagaatgacaggatcagatgtgtggtgtaggcacctctgggggcaactgtacaagatggactgaagggaggccaggtggggactttgctggaatccaggagataaggatgcagccaggaccagcatgaccctcccgcggtggatgtccaggccatccactgggacaccatcaggtcccttgaccgtcacatagccagatgtgtcttcagtgtccccaacttgattgtcagctctgccaggatcgggatcctgagcactggctttcctggatgaatcttttgctgatcttatctcttctctattttccaacctttatctgtcatttatgttttttgttctacttttcacatcttttaaatttttctttcaacgtttctaatatctaatgcaattagccccaaggaaaactaagggaaacaagaaaaaacaagaaaggaaatgtactctcagtacttcagctgtgagctgtgagcttcagctgtgagctggagtgttttgctctggttttaggtttttgattttttttaagaaagagggtcacactctgttgcccaagctggtgagcagtagaatgatataactcactgcaaccttgaactcctgggctcaagagatcctcccaccttggcctcccaaagtgctaggattacaggcacgagccaccgtgcctggcctccaggcatattttatcctttctcaagctatgtgtagtgttttgacaaaaataaattttaaaaagagttaatgtcaaaaaaagtttctgtgacttaagaaggaccactgcttagggtgctcctcctgggtaccaaaacaagaaacagaactttctattccctttatggcctcttgtactttttagttccaccctcctcctgaaaaaatatgatcattttcatcatcatcatcaccatcatcaccattaattacttgttggaaatttaaagcagaattgcctctgaggttggaaggctggagagggcatgagagataggaaacgtcactttcttctgcctgactggccttgtgctctggtgtggggctgtgtctgcccagggggtgtggcctcttttcctgtcttcacatatgaaacatgagctggcaatgccctcatctttaccttgagtttttttgtttgtttgttttgtttttgaagatggagtctcgctcctgtcgcccaggctggagtacagtggcgcatctcagctcactgcaacctccatctcccgggttcaaatgattctcctgcctcagcttcccaagtacctgggactacaggcactcaccaccacacccggctaatttttgtatttttagtagggttttgtcatgttggtcaggctggtctcgatctactgacctctagtgatctgcccgcctcagcctcccaaagtgctgggattacaggcgtgagccaccatgcgtggcttagaattttatacaacacctatgtgttaacttttccaaaaagtaaacatataataaatatgtgcccctcatcaagggaagactttcaagggggagataggccttccaggatgggaaagggcactggacaaaggctcaggactgcgacacccaccacagagccctgcagtgagagtctggttcttcgatgtatttatgtatgtttgtttgtttatttatttatttactttttgagacggagtctcgctctgtcacccaggctggagtgcagtggtgtgatcttggctcactgcaacctccacctcctggattcaagcaattctcctgcctcagcctcccgagtagctgggactacaggtgcctgccatcatgcctggctatttttatatttttagtagagacggggtttcaccatgttggtcaggctagtctcaaattcctgaccttaggtgatccacccgcctcagcctcccgaagtgctgggtttacaggcgtgaagtcaccggcacacccggcctatttatttatttattatttatttatttttgagacagggtcctttctccggttttccagctgaagtgcagtcacacaagtctctgctcactgcaacctgtgcctcctaggctcaagcaatcctcccactcagctctgagtagttggactacaggtccgcggccactgcactgggctaattttgtatttttagtagagatggggtttggccatgttgcccaggctggtcttgagcttctgggcccaagcaattcacccgcctcagcctcccaaagtgccctaattacaggcatgagttaccgtacccggctggctcttggatttaagcccctgcccttcctccttcttactggatattcatgtctctgagccccactgcttcaattaaaacagagaggtatggtcatttctgcctcccagggacttgggatgatagcatttaatcatgaagacagaagtacctatcacagggcctgcctgacttgttcttcctaatgtcaaaaccttggcccaaatgtctactgtgctgaacgtaaggctcctatcagaatgctctcagctaggaactggtcttctggagactctgtggcgtaggatgattcaaccaccttcctagttcttgagtttcagtaataggatctcatagcagttcctgtagtgtgtgagtcacttaagaagacctctggcttccctggaacacaggtaacaaataccttggcttgggatcaagatcctccctacccagggaagggctgagctggccaggacaactgtgtttgggccagagcagcagggtcctgcactctgcagggagcaatcacaggtgggagaagcccacagcctgggatcagaagtgccagaagcttaggaacaggagtcctggggtcccagctttctcgctgtctctccaagccttgagtctttcacctgaaaaattgacatcatcgtgcccacctcagggcccgttgagttaagtcatcgcgtggacttgaagcacctagcacttgtgatcattgaagtcagaaaatgagttcccttctctttcggcgccccattggcaggaagccaaccatcgtcagcccactggcactggaggagcgttgatcatgtgcagagcagatgagtggctactctccctgtcttccaggttttcccagagtggctgtgggatctggagtcggctggtgacagagctgctgggcatcctccactcacccctgttgcctcctgatgagggaaatgggcagagaggagatctgctcacaagttagaccctggcttctctcagtgggcagtgtggacaggggaagggggaaggcagggagcaaaggatgcaggagcaaggagaaaacttccaggctcctcccttccaaagtcacccagccttgagatcattgcagatgcaacaggtgcaaagagaagaacactttgggaccttggaatgcgggaagctggctcagtgttccttccaccctgtgagacatgtgtgacatttttgttgttgttgctgttttgagacggagtttcccccttgttgcccaggctggagtgcagtggcatgatctcggctcaccacaacctctgcttcccaggttcaagtgattttcctgcctcagcctccctagtacaggcatgtgccacaacgcctggctaattttgtatttttagtagagatagggtttctccatgttggtcaggctggtctcaaactcccaacctcaggtcgatctgcctgccttggcctgccaaagtgctgggattaccagcatgaacctccatacccgacctatgtgacattcttaaggtccacgaaagggctggtggttggaggccagagggtggcctgtgagatactgggattggaagagtaggtggcctgtgggtgtctcctctggggggagactccccagggggaggggttcctcctctccaaacagtagctcagcacagggacagtaacagtaatgggggcatgtgtgtaaggaatgctttcccaggccccctcttcataggtgtttgcagcagcatctcacaatagccctaagaaggttaaatgttgcacatcccatcctacagaagtggaaatcgagcctcagagaagttgtgtcgcttttggggctatcttcccctgtagagtgtgactccatcgcctcctgctttaccaggtgctgagaacctctaatcatctcccatggatttgtgatcagcgttgcagctctcccagcagccctggacagtggtgagtcccctcagctggccgggacagtcctctgctctcacctctctgcttctctggctccatcccagctccgcctcagctggtctccttgcaaacccacagggctccaggacatcctccctctggagcccatgctgccttcagcacttcaccccctaccggtgatggcaacaattggtgttctttgtccactttatttattggctcatttttttgtttttgagaaagagtctcactctattgcccaggctggagtgcagtggcataatctcagctcactgcaacctctgcctcccgggttcaagcgattttcacgtcttcagcctaccaactagctgagactacaggacatgcaccaccatgcccagctaatttttgtattttttttggtaagatggggttttaccatgttggccaggttggtgtcgaactcctgacttcaggtgatctgcccacctcaacctcccaaattgctaggattacaggcatgagccactgtgcctggacttatttgctcattttgatatgtactgtatgttttgagtcagagtctcactctatcgcccaagctggagtgcagtggcatgatcaaagctcactgcaacctcaaactcctgggctcaagcagtcctcccacctcagcctcccgaatagcagggaccacaggcgcactccaccacacctggctttcctttttttgttttttttaatgtttttgtagagacaggcaggctatccatatgttgcccaggctggtctcaaacttttcgcctcaaggtatcctcccacctcagcctcctaaagtgctgggattacaggcttgaatcccaggctcgcatcctgcctatttgcttcttttcttttcttttcttttctttttttttttttttggaatgaagttttgctcttgcccaggctggagtgcaatggcgcgatctcgtctcactacaacctctgcctcccgggttcaagcgattctcctgcctcagcctcctgagtagctggggattacaggtgcccgctaccacgcccggctaatttttgtatacttagtagacatgatgtttcaccatgttgaccaagctggtcttgaacttctgacttcaggtgatccacccacctcggcctcccaaagtgctgggattacaggcgtgagccaccacgcccagcgcctatttgcttattttcaataagaaaagttattttccataagaaaagttatacctgtctatttaaaaaaaaaaaaaatcatacactgcagaagcacacgctgtagcaagggcactcttcagttagggacattactacatgtctctcctgtccatccaggatattcaaacatgttcccattttccaataatccacttaaatcctgtattatgtgctggtatcatctcccttttgcagatgggtaaactgagtcacggggcgatccaatattttgttcaagatcacaggctatgtatgtcagtgtagtgtgggagattaaaaaaggaaaaagaccacaggctacggagtggaagagcctcagggagcctggccgtgctgtgacatccaccagagcgcccatcagttcaacaacctaaaagagtttccttacaggaattcttaagcaaaagacaggaattccgtttgaaatatatttccctctccaaatagacaatggtaatgtgggtaggtagaactaagaaggtaggactcacattaagaaggtaggactcacattaagaaggtaggactcacatgtagacagatttccgaccgcattctgcatttgtacttttgctctcagctagactggatcctgagttatgtcttaaggccacatttccgggagagctccccactgagaccatcatgttcatgggatgctcgggtctggtggtagaatctgccccctgcgggcactgggcagtggggtacgggatgggcgtgcaggctgcagcccctaaccgctgcgctctcctccctaaggcccccagcagtcagcatgtggctgccgcgcgtctccagcacagcagtgaccgcgctcctcctggcgcagaccttcctcctcctctttctggtttcccggccagggccctcgtccccagcaggcggcgaggcgcgcgtgcatgtgctggtgctgtcctcgtggcgctcgggctcgtccttcgtgggccaactcttcaaccagcaccccgacgtcttctacctaatggagcccgcgtggcacgtgtggaccaccctgtcgcagggcagcgccgcaacgctgcacatggctgtgcgcgacctggtgcgctccgtcttcctgtgcgacatggacgtgtttgatgcctatctgccttggcgccgcaacctgtccgacctcttccagtgggccgtgagccgtgcactgtgctcgccacccgcctgcagtgcctttccccgaggcgccatcagcagcgaggccgtgtgcaagccactgtgcgcgcggcagtccttcaccctggcccgggaggcctgccgctcctacagccacgtggtgctcaaggaggtgcgcttcttcaacctgcaggtgctctacccgctgctcagcgaccccgcgctcaacctacgcatcgtgcacctggtgcgcgacccgcgggccgtgctgcgctcccgggagcagacagccaaggctctggcgcgtgacaacggcatcgtgctgggcaccaacggcacgtgggtggaggccgaccccggcctgcgcgtggtgcgcgaggtgtgccgtagccacgtacgcatcgccgaggccgccacactcaagccgccaccctttctgcgcggccgctaccgcctggtgcgcttcgaggacctggcgcgggagccgctggcagaaatccgtgcgctctacgccttcactgggctcagtctcacgccacagctcgag

(19) United States
(12) Patent Application Publication
(10) Pub. No.: US 2002/0061562 A1
(43) Pub. Date: May 23, 2002

(54) Methods of treating macular corneal dystrophy

(76) Inventors: Michiko N. Fukuda, San Diego, CA (US); Tomoya O. Akama, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/927,602

(22) Filed: Aug. 9, 2001

Related U.S. Application Data
(60) Provisional application No. 60/325,773, filed on Aug. 11, 2000.

(51) Int. Cl. C12P 19/28; C12P 21/02; C12N 5/06; C08B 37/00; C12N 9/10; C07H 21/04

(52) U.S. Cl. 435/85; 435/193; 536/54; 536/23.2; 435/320.1; 435/325; 435/69.1

(57) ABSTRACT

The invention provides an isolated polypeptide encoding a corneal N-acetylglucosamine-6-sulfotransferase (GlcNAc6ST) or active fragment thereof, where the GlcNAc6ST or active fragment thereof catalyzes sulfation of keratan sulfate. The present invention also provides a method of treating a subject with macular corneal dystrophy. The method includes the steps of administering to the subject an effective amount of an agent that increases expression or activity of a GlcNAc6ST, whereby the amount of sulfated keratan sulfate in the cornea of the subject is elevated. A method of the invention can be used to treat macular corneal dystrophy type I or type II.

This application is based on, and claims the benefit of, U.S. Provisional Application No. ______ (yet to be assigned), filed Aug. 11, 2000, which was converted from U.S. Ser. No. 09/638,211, and entitled METHODS OF TREATING MACULAR CORNEAL DYSTROPHY and which is incorporated herein by reference.

This application was made with government support under CA71932 awarded by the National Institute of Health. The government has certain rights in the invention.

### BACKGROUND OF THE INVENTION

1. FIELD OF THE INVENTION

The invention relates generally to ophthalmology and keratan sulfate biology and, more specifically, to identification of a novel corneal keratan sulfate sulfotransferase involved in macular corneal dystrophy.

2. BACKGROUND INFORMATION

Blindness, which afflicts nearly 40 million people worldwide, can be caused by trauma, infection or genetic inheritance. While trauma and infection can be prevented or at times treated, relatively few options are available to prevent or treat blindness that results from a genetic predisposition.

One hereditary cause of blindness is macular corneal dystrophy (MCD; MIM 217800), an autosomal recessive disease that develops in the cornea of both eyes as diffuse grayish white spots (opacities) with indistinct edges, and progressively leads to severe visual impairment. Macular corneal dystrophy appears in the first decade of life and affects the central portion of the anterior layers of the stroma. By the third decade, the diffuse stromal opacity with its ground glass appearance extends posteriorly to the endothelium and laterally to the limbus. Within the stroma are small irregular, white patches that continue to expand, enlarge, and become more confluent. Late in the course of the disease, Descement's membrane becomes opacified and there are endothelial guttate changes.

Histopathologically, the opacities are accumulations of glycosylaminoglycans within the endoplasmic reticulum which are thought to accumulate because of an inability to break down corneal keratan sulfate.

MCD is classified into two principal subtypes, type I and type II. Type I MCD is more prevalent and is characterized by the absence of antigenic keratan sulfate in the cornea, serum, and cartilage due to a genetic defect resulting in production of abnormal keratan-aminoglycan. In contrast to patients with type I MCD, keratan sulfate is detectable in the serum of patient's with type II MCD, although the level can be below normal levels. Both types of macular corneal dystrophy exhibit clinically similar phenotypes in the cornea.

The established treatment of macular corneal dystrophy is penetrating keratoplasty. However, the disease can recur in the graft, with the recurrence rate estimated to be inversely proportional to the graft size. Excimer laser phototherapeutic keratectomy has been attempted, but post-treatment vision may still be clouded by residual diffuse stromal haze (Wu et al. Arch. Ohthalmol. 109:1426-1432 (1991)). Recent preliminary results with phototherapeutic keratectomy have been more promising (Wagoner and Badr, J. Refract. Sura. 15:481-484 (1999)).

While the catabolism of keratan sulfate is understood, little is known concerning the biosynthesis of this important component of both cornea and cartilage. To date, chromosome 16 has been linked to MCD type I (Vance et al., Am. J. Human Genet. 58:757-762 (1996); Lui, et al., Brit. J. Ophthalmol. 82:241-244 (1998)). However, the molecular defect underlying macular corneal dystrophy types I and II, which would provide the basis for dramatic improvements in genetic testing, treatment and prevention of this disease, remains to be identified.

Thus, there exists a need to determine the genetic cause of macular corneal dystrophy and to develop less radical options for the treatment and prevention of this disease. The present invention satisfies this need and provides additional advantages as well.

### SUMMARY OF THE INVENTION

The present invention provides an isolated nucleic acid molecule which contains a sequence encoding a corneal N-acetylglucosamine-6-sulfotransferase (GlcNAc6ST) or active fragment thereof, where said GlcNAc6ST or active fragment thereof catalyzes the sulfation of keratan sulfate. An isolated nucleic acid molecule of the invention can have, for example, substantially the amino acid sequence of SEQ ID NO: 2, and can contain SEQ ID NO: 1 or a portion thereof. In one embodiment, the sulfation of keratan sulfate produces sulfated keratan sulfate immunoreactive with antibody 5D4. In another embodiment, the sulfation of keratan sulfate produces sulfated keratan sulfate hydrolyzable by keratanase.

The invention further provides a vector such as a mammalian expression vector, that contains a nucleic acid molecule encoding a corneal GlcNAc6ST or active fragment thereof, where the GlcNAc6ST or active fragment thereof catalyzes sulfation of keratan sulfate. Host cells that contain a vector of the invention also are provided.

The invention further provides an oligonucleotide which contains a nucleotide sequence having at least 10 contiguous nucleotides of SEQ ID NO: 1 or SEQ ID NO: 38, or a nucleotide sequence complementary thereto, provided that the oligonucleotide does not consist of GenBank accession number AI824100. Such an oligonucleotide can have, for example, at least 15 contiguous nucleotides of SEQ ID NO: 1 or SEQ ID NO: 38, or a nucleotide sequence complementary thereto.

The invention also provides an isolated polypeptide encoding a corneal N-acetylglucosamine-6-sulfotransferase (GlcNAc6ST) or active fragment thereof, where the GlcNAc6ST or active fragment thereof catalyzes sulfation of keratan sulfate. An isolated corneal GlcNAc6ST of the invention can have, for example, substantially the amino acid sequence of SEQ ID NO: 2.

Further provided by the invention is substantially purified antibody material that specifically binds a corneal GlcNAc6ST that catalyzes sulfation of keratan sulfate. The substantially purified antibody material can specifically binds a GlcNAc6ST having, for example, the amino acid sequence SEQ ID NO: 2. In one embodiment, the substantially purified antibody material is monoclonal antibody material.

The present invention also provides a method of treating a subject with macular corneal dystrophy. The method includes the steps of administering to the subject an effective amount of an agent that increases expression or activity of a GlcNAc6ST, whereby the amount of sulfated keratan sulfate in the cornea of the subject is elevated. A method of the invention can be used to treat macular corneal dystrophy type I or type II. In one embodiment, the expression or activity of an endogenous GlcNAc6ST is increased. In another embodiment, the expression or activity of human corneal GlcNAc6ST or murine GlcNAc6ST is increased. In further embodiments, the agent is, for example, a nucleic acid molecule encoding a GlcNAc6ST, or active fragment thereof that catalyzes the sulfation of keratan sulfate, or is a GlcNAc6ST polypeptide or active fragment thereof. In yet another embodiment, an agent useful in the invention increases transcription of a GlcNAc6ST that catalyzes the sulfation of keratan sulfate and can, for example, selectively increase transcription of GlcNAc6ST in the cornea of the subject.

The invention further provides an ex vivo method of treating a subject with macular corneal dystrophy. In a method of the invention, an effective amount of an agent that increases expression or activity of a N-acetylglucosamine-6-sulfotransferase is administered in vitro to primary, explanted corneal cells; and these cells are introduced into the cornea of the subject, whereby the amount of sulfated keratan sulfate is elevated in the cornea of the subject.

The invention further provides a method of monitoring therapeutic efficacy in a subject being treated for macular corneal dystrophy. The method includes the steps of obtaining a test sample from the subject; determining a sample level of expression or activity of GlcNAc6ST in the test sample; and comparing the sample level to a reference level from the subject; whereby an increase in the sample level relative to said reference level is indicative of productive therapy. In such a method of the invention, the sample level can be measured, for example, using an antibody that specifically binds GlcNAc6ST. The sample level also can be measured, for example, using a nucleic acid molecule that specifically hybridizes to SEQ ID NO:1 or SEQ ID NO: 38.

The invention further provides a method of determining susceptibility to macular corneal dystrophy in an individual. The method includes the step of determining the presence or absence in an individual of a MCD-associated allele linked to a corneal GlcNAc6ST locus, where the presence of the MCD-associated allele indicates susceptibility to MCD in said individual. A method of the invention can be useful, for example, to diagnose type I MCD or type II MCD. In a method of the invention, the MCD-associated allele can be localized, for example, within a corneal GlcNAc6ST gene such as within a corneal GlcNAc6ST coding region. In one embodiment, the MCD-associated allele is one of the following mutations of SEQ ID NO: 1: deletion of the entire open reading frame, a frameshift mutation nucleotide 1106, 1213A→G, 1301C→A, 1512G→A, 1323C→T or 840C→A. In another embodiment, the MCD-associated allele occurs within the region coding the 3′-phosphate binding domain of corneal GlcNAc6ST such as 203D→E and 211R→W in SEQ ID NO: 2. In yet a further embodiment, the MCD-associated allele occurs within a corneal GlcNAc6ST 5′ regulatory region such as CHST6.

### BRIEF DESCRIPTION OF THE DRAWINGS

FIG. 1 shows the human corneal N-acetylglucosamine-6-sulfotransferase nucleic acid sequence (SEQ ID NO:1) and predicted amino acid sequence (SEQ ID NO:2).

FIG. 2 shows an alignment of amino acid sequences encoding human corneal N-acetylglucosamine-6-sulfotransferase (SEQ ID NO:2; hC-GlcNAc6ST), human intestinal N-acetylglucosamine-6-sulfotransferase (SEQ ID NO:4; hI-GlcNAc6ST), and mouse intestinal N-acetylglucosamine-6-sulfotransferase (SEQ ID NO:5; mGlcNAc6ST). The consensus sequence is shown above as SEQ ID NO: 3.

FIG. 3A shows the radiation hybrid map of the human CHST6 locus. The horizontal line represents a part of chromosome 16q22, positioned with centromere to the left and q-telomere to the right. Locations and directions of the genes encoding I- and C-GlcNAc6ST are shown by arrows and arrowheads. Distances between each marker correspond to Stanford G3 radiation hybrid map version 2.0. FIG. 3B shows genomic structures of CHST5 and CHST6. Directions of each gene are indicated by arrows. Exons and coding regions are shown in open and gray boxes, respectively. Hatched boxes indicate upstream regions which are enlarged and shown in FIG. 3A. FIG. 3C shows an alignment of a portion of C-GlcNAc6ST (human corneal GlcNAc6ST; SEQ ID NO: 6) with other sulfotransferases: I-GlcNAc6ST (human intestinal GlcNAc-6-sulfotransferase; (SEQ ID NO: 7; Lee et al., Biochem. Biophys. Res. Commun. 263:543-549 (1999)), HEC-GlcNAc6ST (human high-endothelial-cell GlcNAc-6-sulfotransferase; SEQ ID NO: 8; Bistrup et al., J. Cell Biol. 145:899-910 (1999)), GlcNAc6ST (human GlcNAc-6-sulfotransferase; SEQ ID NO:9; Uchimura et al., J. Biochem. 124:670-678 (1998)), KSG6ST (human KS Gal-6-sulfotransferase; SEQ ID NO: 10; Fukuda et al., J. Biol. Chem. 272:32321-32328 (1997)); and Ch6ST (human chondroitin-6-sulfotransferase; SEQ ID NO: 11; Fukuda et al., Biochim. Biophys. Acta 1399:57-61 (1998)). Clustal W version 1.7 (Thompson et al., Nucleic Acids Res. 22:4673-4680 (1994)) was used for multiple alignment of amino acid sequences. Highlighted letters and letters on gray background represent identical and conserved amino acids, respectively. Double underlines indicate 5′- and 3′-phosphate binding domains reported previously (Kakuta et al., Nature Struct. Biol. 4:904-908 (1997), and Kakuta et al., Trends Biochem. Sci. 23:129-130 (1998)). Mutated amino acids found in C-GlcNAc6ST in MCD patients are marked by asterisks.

FIG. 4 shows the distribution of sulfated KS and CHST6 transcripts in human normal and MCD type II corneas. Semiserial tissue sections of normal cornea (A-L) and MCD type II cornea (M-U) were sequentially analyzed by immunohistochemistry for sulfated keratan sulfate (A-D and M-O) and in situ hybridization for CHST6 mRNA (E-L and P-U). Corneal endothelial cells were not included in the MCD type II sample. The clefts in the stroma are artifacts due to tissue processing. The corneal epithelial cells (B, F, J, N, Q and T), stroma (C, G, K, 0, R and U) and endothelial cells (D, H and L) are shown under high magnification. Immunostaining was performed with anti-sulfated KS antibody, 5D4, in A-D and M-O. In situ hybridization was performed with CHST6 anti-sense probe (E-H, P-R) and sense probe (I-L and S-U). Bar in S=200 μm, and bar in U=50 μm.

FIG. 5 shows MCD type I mutations in a representative family. Boxed haplotypes shown under a pedigree of MCD family represent disease-associated haplotypes. PCR-RFLP analysis is also shown under each family member. Sequence chromatograms of the mutated region of CHST6 in normal and MCD families are shown at the right. The mutated nucleotide and the substituted amino acid shown under sequence chromatograms are underlined. PCR-RFLP analysis confirmed segregation of the CHST6 mutation in this family.

FIG. 6 shows DNA rearrangements found in the upstream region of CHST6 in MCD type II patients. FIG. 6A shows an illustration of homologous regions located upstream of CHST5 and CHST6, represented as hatched boxes in FIG. 3B. Homologous upstream regions A and B in each gene are shaded. Exon 1 is marked for each gene. Gray arrows show Alu repetitive sequences. Open arrowheads indicate PCR primers used for detection of DNA rearrangements in MCD type II patients. Black box shows a probe used for Southern blot analysis. FIG. 6B shows homozygous replacement found in two MCD type II patients. Boxed haplotypes indicate homozygosity in these patients. PCR reactions were performed with genomic DNA from normal individuals and patients using the primers shown in FIG. 6A. FIG. 6C shows an MCD family with both type I and type II mutations. Haplotypes with gray background indicate the missense mutation (R50C, Table 1) classified as type I. FIG. 6D shows an MCD type II family with a deletion mutation found upstream of CHST6. Genomic DNAs from patients and unaffected family members were digested by SpeI. Southern blot analysis shows a lack of positive bands in lanes with patient samples; conversely, bands are apparent in lanes representing unaffected individuals. By genomic PCR analysis, junction of this large deletion was identified on homologous region B shown in FIG. 6A.

FIG. 7 shows distinct types of mutations within CHST6 are associated with MCD type I and MCD type II. FIG. 7A shows mutations which affect enzymatic activity of C-GlcNAc6ST, such as missense mutations and frame shift mutations, can inactivate C-GlcNAc6ST in not only the cornea but also in other tissues, resulting in a lack of serum sulfated KS. FIG. 7B shows that mutations in the gene regulatory region of CHST6 abolish expression of C-GlcNAc6ST in corneal cells but not in other tissues, resulting in the presence of sulfated KS in serum.

### DETAILED DESCRIPTION OF THE INVENTION

Genetic lesions can result in certain forms of blindness that are difficult to prevent or treat. One hereditary form of blindness is macular corneal dystrophy, in which the cornea becomes progressively more opaque. Macular corneal dystrophy is characterized biochemically by the presence of abnormally sulfated keratan sulfate in the cornea (Nakazawa et al., J. Biol. Chem. 259:13751-13757 (1984); Klintworth et al., Ophthalmic Pediatr. Genet. 7:139-143 (1986); Thonar et al., Am. J. Ophthalmol. 102:561-569 (1986); and Edward et al., Ophthalmology 97:1194-1200 (1990)). While macular corneal dystrophy is characterized by a lack of normal keratan sulfate in the cornea, sulfated keratan sulfate is present in the serum of some MCD patients. Macular corneal dystrophy can be grouped into two types: type II MCD patients contain sulfated keratan sulfate in their serum, while it is and absent in the serum of type I MCD patients (Yang et al., Am. J. Ophthalmol. 106:65-71 (1988) and Edward et al., Arch. Ophthalmol. 106:1579-1583 (1988)). In spite of some knowledge regarding the biochemical defect, the underlying molecular lesion responsible for the abnormally sulfated keratan sulfate in the cornea of MCD patients has yet to be identified.

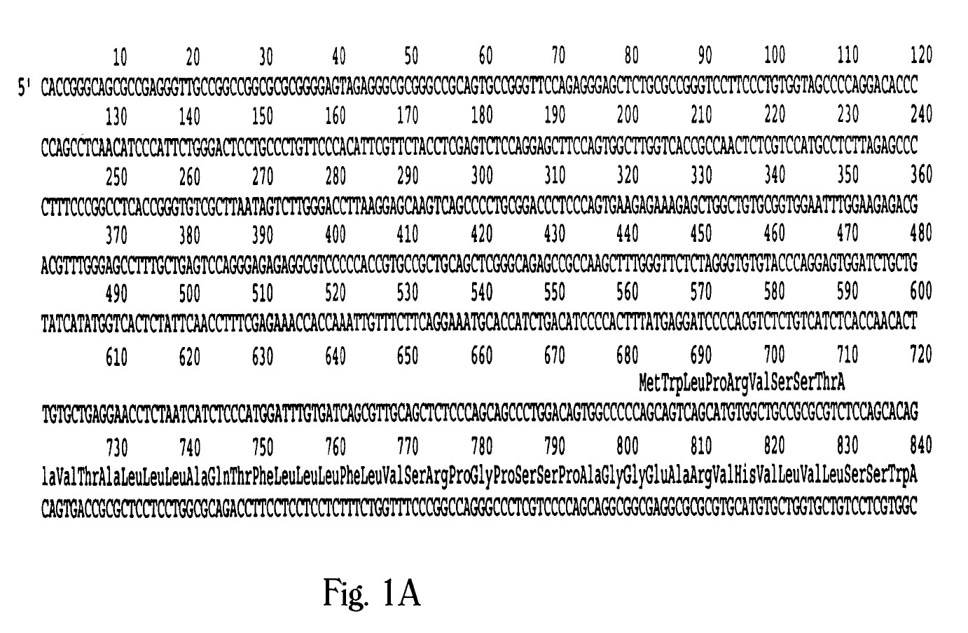

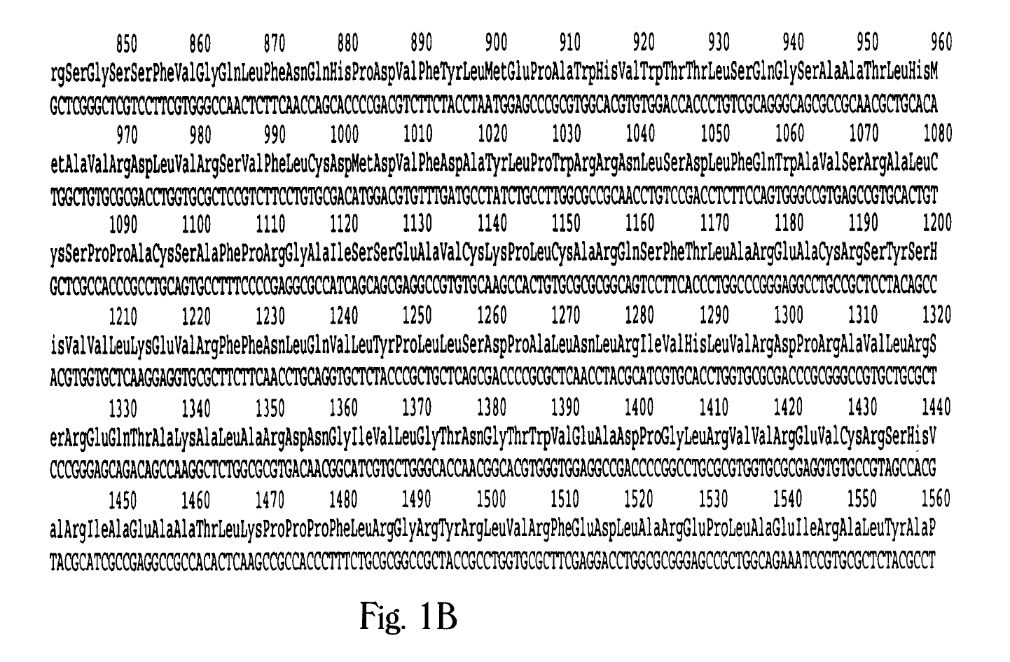

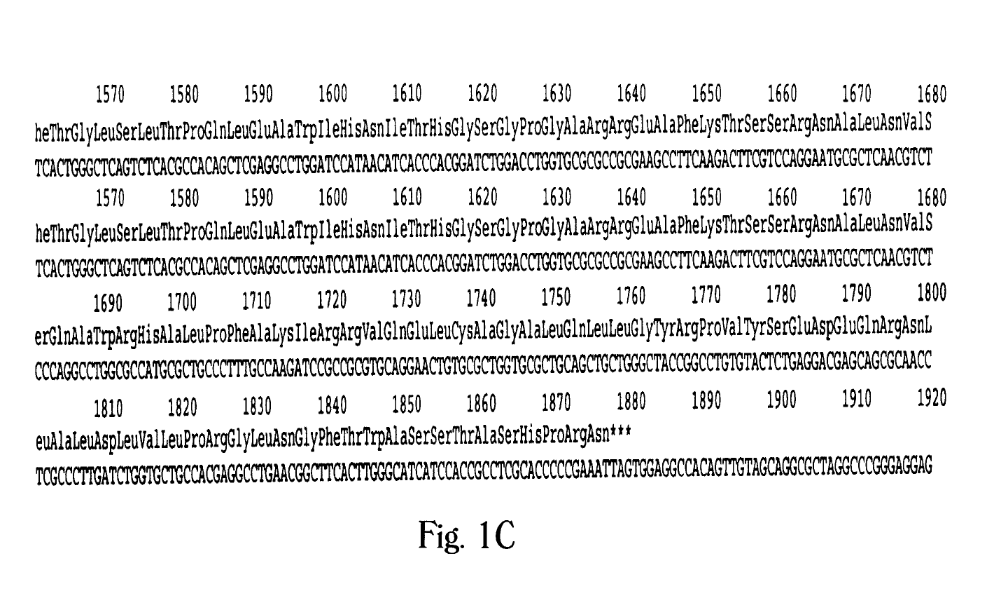

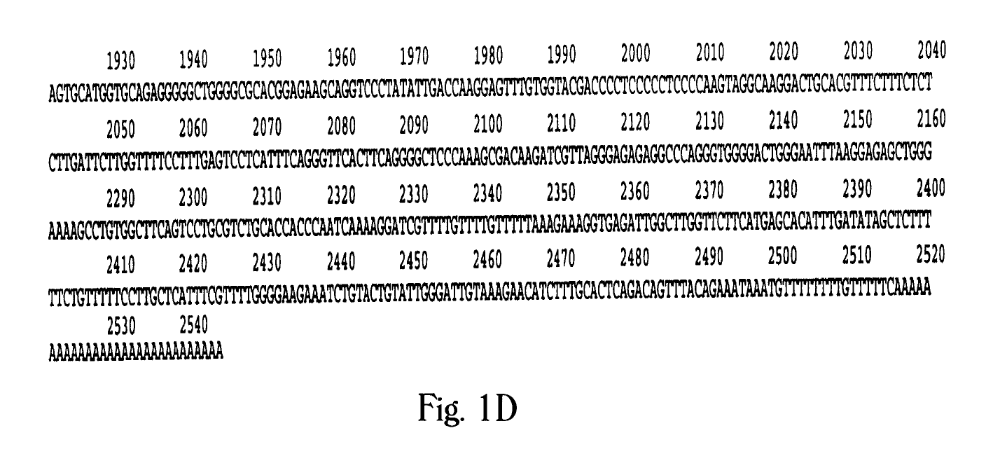

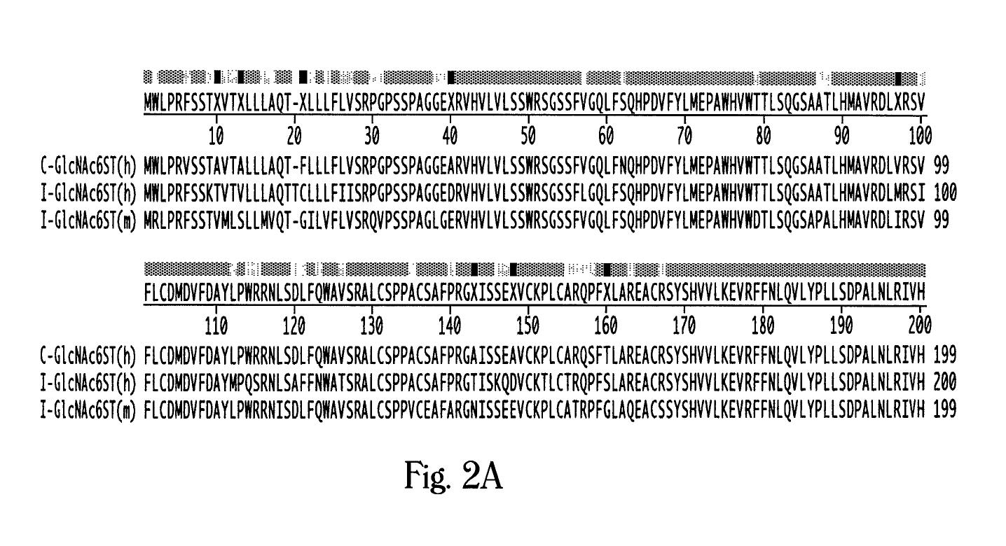

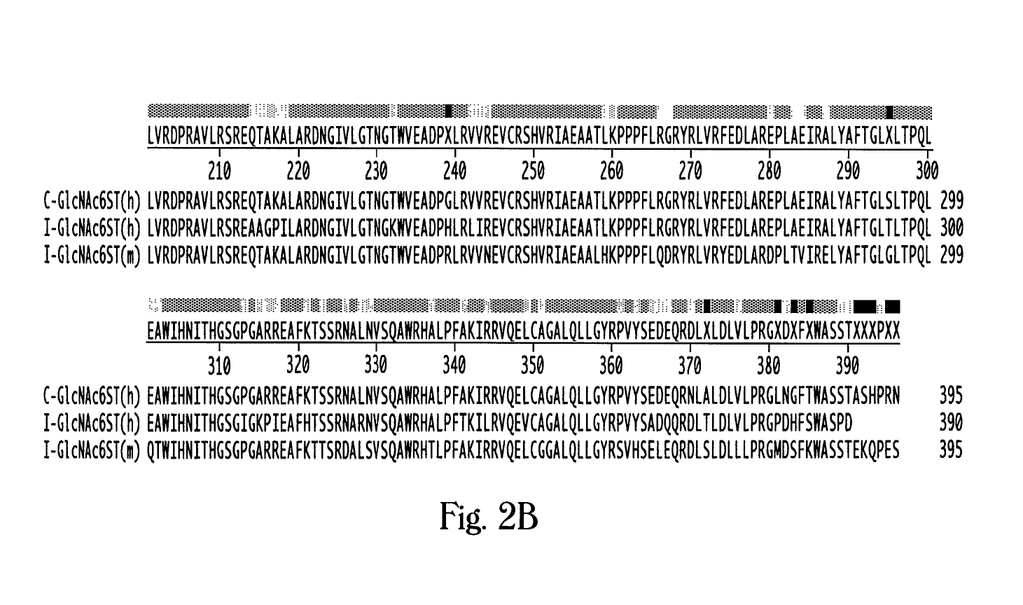

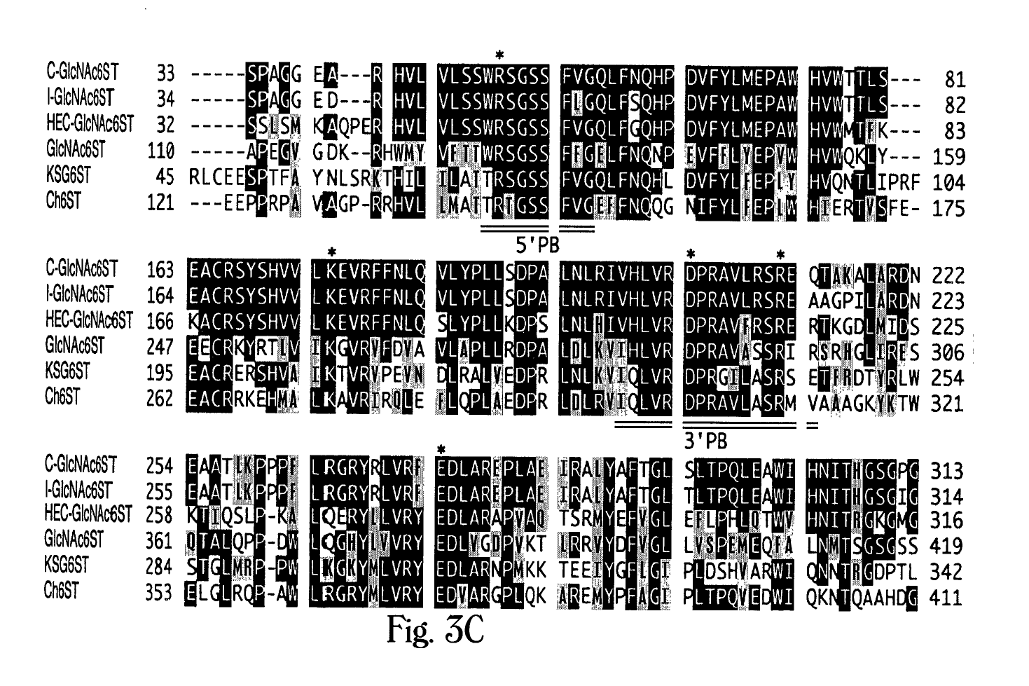

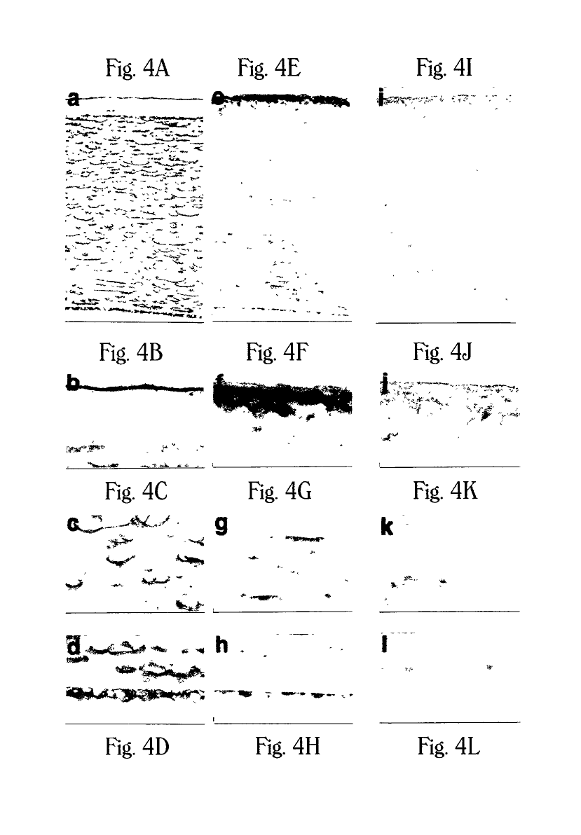

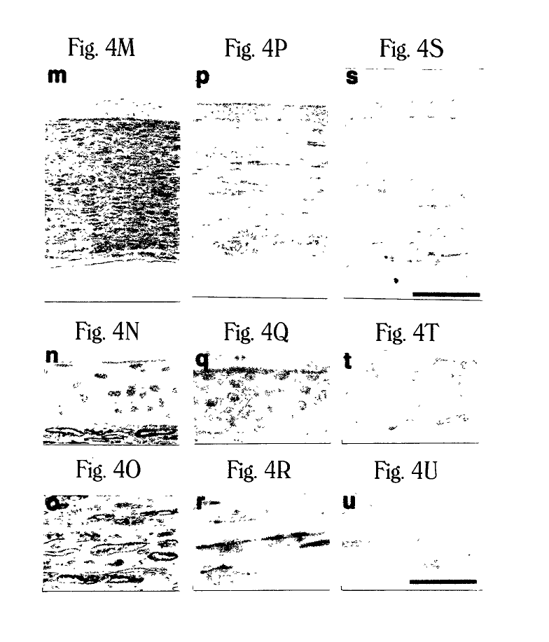

The present invention is directed to the exciting discovery of a novel human N-acetylglucosamine-6-sulfotransferase and of the determination that genetic lesions in the gene encoding this enzyme result in macular corneal dystrophy. The novel sulfotransferase, which is encoded by the CHST6 gene, has been designated corneal N-acetylglucosamine-6-sulfotransferase (C-GlcNAc6ST, SEQ ID NO:2) and is shown in FIG. 1, and an alignment of the encoded protein with human intestinal GlcNAc6ST and murine intestinal GlcNAc6ST is shown in FIG. 2. Furthermore, as disclosed in Example III, in situ hybridization using labeled oligonucleotide probes specific for CHST6 demonstrated that C-GlcNAc6ST is expressed in normal human cornea, but not in the cornea of type II MCD patients, and that sulfated keratan sulfate is detected by the anti-sulfated keratan sulfate antibody 5D4 in normal cornea but not in the cornea of type II patients (see FIG. 4). These results indicate that expression of C-GlcNAc6ST (SEQ ID NO:2) is correlated with that of sulfated KS in human cornea.

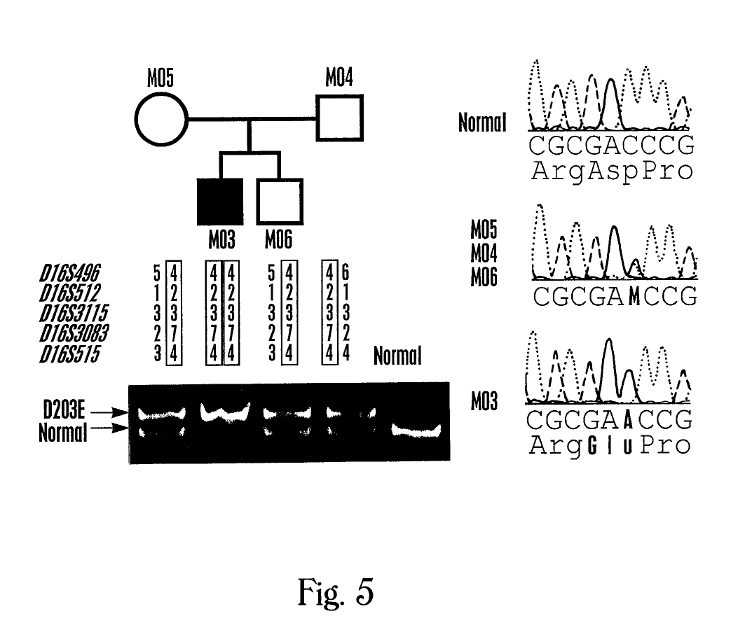

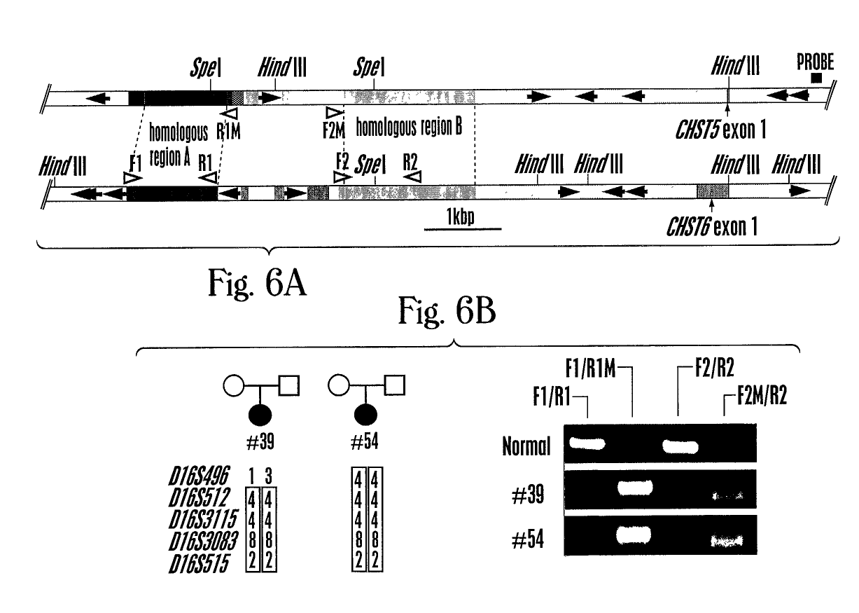

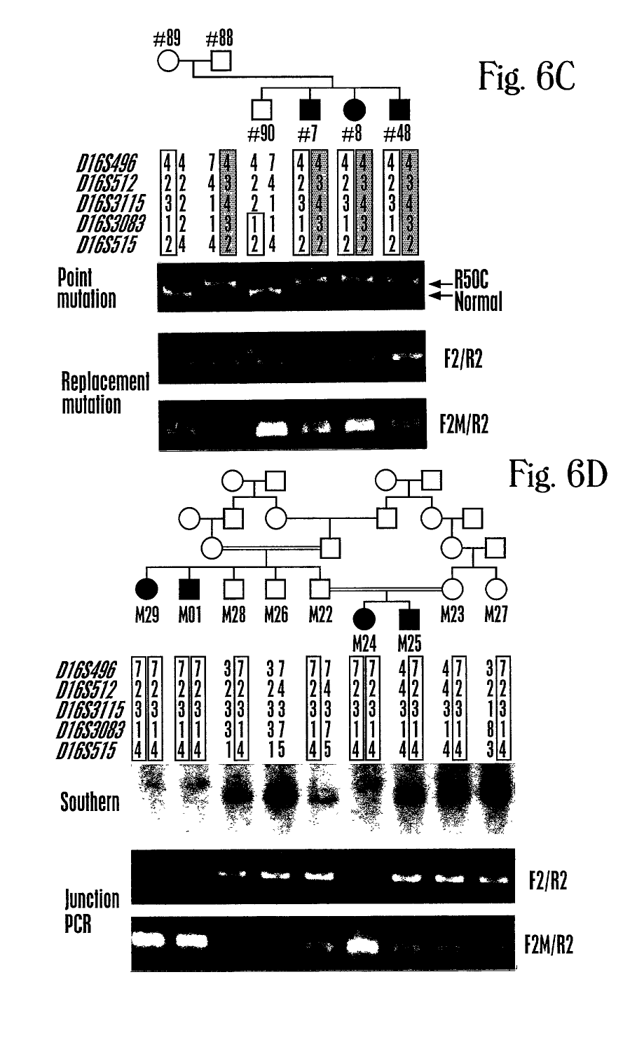

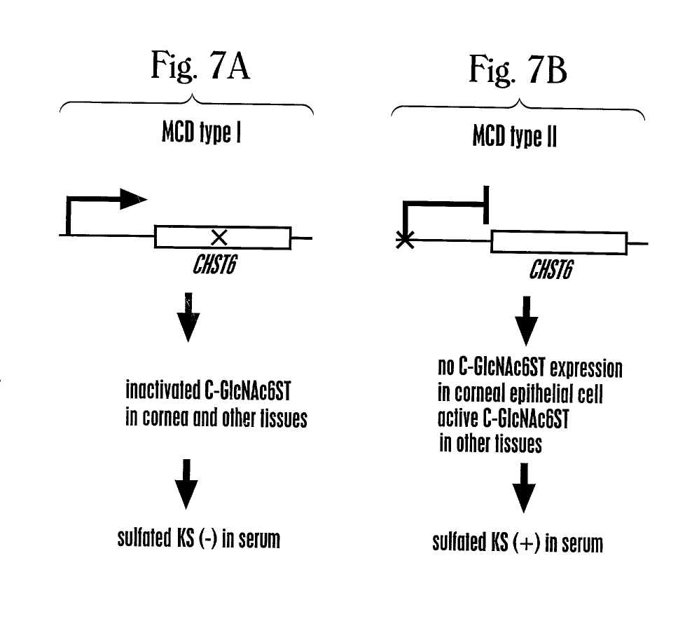

As further disclosed herein in Example VI, sequencing analysis and restriction fragment length polymorphism analysis of type I MCD patients revealed inactivating mutations in the coding sequence of the novel corneal N-acetylglucosamine-6-sulfotransferase, including deletions, frame-shifting insertions and missense mutations (see Table 1 and FIG. 5). Furthermore, southern blot analysis and polymerase chain reaction (PCR) analysis disclosed in Example V demonstrated that regions upstream of the CHST6 gene are altered or missing in type II MCD patients (Table 1 and FIG. 6). A nearby carbohydrate sulfotransferase gene, CHST5, contains an upstream region homologous to the upstream region of CHST6 (see Example II and FIG. 6A); and the identified alterations and deletions in MCD type II patients are localized to these homologous regions (Example V). These results indicate that the lack of functional C-GlcNAc6ST expression in cornea is well correlated with macular corneal dystrophy and further indicate that C-GlcNAc6ST coding region mutations are associated with type I MCD while regulatory region mutations are associated with type II MCD.

As further disclosed herein in Example VI, a C-GlcNAc6ST encoding nucleic acid was transfected into HeLa cells, which normally do not produce sulfated keratan sulfate, and, when keratan sulfate was provided as the substrate, sulfated keratan sulfate was detected using the 5D4 antibody (Table 2). This result demonstrates that genetically engineered expression of C-GlcNAc6ST can be sufficient to produce sulfated keratan sulfate, thus correcting the biochemical defect underlying macular corneal dystrophy. These discoveries provide a basis for novel methods of diagnosing and predicting susceptibility to macular corneal dystrophy, and for gene therapy to treat this disorder.

Thus, the present invention provides an isolated polypeptide that encodes a corneal N-acetylglucosamine-6-sulfotransferase (GlcNAc6ST) or active fragment thereof that catalyzes sulfation of keratan sulfate. Such a corneal GlcNAc6ST can have, for example, substantially the amino acid sequence of human SEQ ID NO:2 as shown in FIG. 1.

As used herein, the term “isolated” means a polypeptide or nucleic acid molecule that is in a form that is relatively free from contaminating lipids, polypeptides, nucleic acids or other cellular material normally associated with the polypeptide or nucleic acid molecule in a cell.

The term “N-acetylglucosamine-6-sulfotransferase” or “GlcNAc6ST,” as used herein, means an enzyme that catalyzes the addition of a sulfate ester to keratan sulfate when expressed in cornea. Preferably, a GlcNAc6ST catalyzes the addition of a sulfate ester to carbon 6 of N-acetylglucosamine of keratan sulfate I in cornea. Similarly, the phrase “catalyzes sulfation of keratan sulfate,” as used herein, means the enzymatic addition of a sulfate ester to keratan sulfate to form sulfated keratan sulfate, preferably addition of a sulfate ester to carbon 6 of N-acetylglucosamine of keratan sulfate I. The presence of sulfated keratan sulfate can be determined, for example, using an anti-sulfated keratan sulfate antibody such as 5D4, available from Seikagaku Co. (Falmouth, Mass.), as disclosed herein in Example VI. In one embodiment, the ability of a GlcNAc6ST to catalyze sulfation of keratan sulfate is determined by expression of the polypeptide in HeLa cells. Human corneal GlcNAc6ST (SEQ ID NO: 2) and murine intestinal GlcNAc6ST (SEQ ID NO: 5), which in HeLa cells produce sulfated keratan sulfate detectable with 5D4 antibody are both GlcNAc6STs that catalyze sulfate of keratan sulfate as defined herein. In contrast, human intestinal N-acetylglucosamine-6-sulfotransferase (SEQ ID NO:4), which does not catalyze sulfation of keratan sulfate when expressed in HeLa cells (see Example VI), is not a “GlcNAc6ST” as defined herein. A GlcNAc6ST of the invention generally has at least 50% amino acid sequence identity to human corneal GlcNAc6ST (SEQ ID NO:2), and can have 55%, 60%, 65%, 70%, 75%, 80% or more % sequence identity to human GlcNAc6ST (SEQ ID NO:2). Percent amino acid identity can be determined using Clustal W version 1.7 (Thompson et al., Nucleic Acids Res. 22:4673-4680 (1994)).

The nucleic acid molecules and polypeptides of the invention encode a corneal GlcNAc6ST. The term “corneal GlcNAc6ST” as used herein, means a GlcNAc6ST that is structurally similar to human corneal GlcNAc6ST and that functions as a GlcNAc6ST to catalyze the sulfation of keratan sulfate. Such a corneal GlcNAc6ST has 90% or more sequence identity to human corneal GlcNAc6ST (SEQ ID NO:2), and can have 91%, 92%, 93%, 94%, 95%, 96%, 97%, 98%, or 99% or more sequence identity to human GlcNAc6ST (SEQ ID NO:2). Percent amino acid identity can be determined using Clustal W version 1.7 (Thompson et al., supra, 1994). In view of the above, murine intestinal GlcNAc6ST (SEQ ID NO:4), which shares 88.1% amino acid identity with human corneal GlcNAc6ST (SEQ ID NO:2), is not a “corneal GlcNAc6ST” as defined herein. As set forth above, human intestinal GlcNAc6ST (SEQ ID NO:4) is not a “GlcNAc6ST” as defined herein, and, similarly is not a “corneal GlcNAc6ST” as defined herein.

Thus, it is clear to the skilled person that the term “corneal GlcNAc6ST” encompasses polypeptides with one or more naturally occurring or non-naturally occurring amino acid substitutions, deletions or insertions as compared to SEQ ID NO: 2, provided that the peptide has at least 90% amino acid identity with SEQ ID NO: 2 and encodes an enzyme that catalyzes the sulfation of keratan sulfate. A corneal GlcNAc6ST can be, for example, a naturally occurring variant of human corneal GlcNAc6ST (SEQ ID NO: 2), a species homolog such as a primate corneal GlcNAc6ST, a GlcNAc6ST mutated by recombinant techniques, and the like.

Modifications to SEQ ID NO: 2 that are encompassed within the invention include, for example, an addition, deletion, or substitution of one or more conservative or non-conservative amino acid residues; substitution of a compound that mimics amino acid structure or function; or addition of chemical moieties such as amino or acetyl groups. The activity of a modified GlcNAc6ST polypeptide or fragment thereof can be assayed, for example, by transfecting an encoding nucleic acid molecule into HeLa cells and assaying for the presence of sulfated keratan sulfate, for example, by immunoreactivity to the 5D4 antibody, as disclosed herein.

A particularly useful modification of a GlcNAc6ST polypeptide of the invention, or active fragment thereof, is a modification that confers, for example, increased stability. Incorporation of one or more D-amino acids is a modification useful in increasing stability of a polypeptide or polypeptide fragment. Similarly, deletion or substitution of lysine can increase stability by protecting against degradation.

The human GlcNAc6ST of the invention catalyzes the sulfation of keratan sulfate. Keratan is a proteoglycan found in tissue such as cartilage and cornea. Typically, keratan sulfate referred to herein means type I keratan sulfate located in cornea. “Normal” keratan sulfate, also referred to herein as “sulfated” keratan sulfate, means wild type keratan sulfate I that is sulfated on carbon 6 of N-acetylglucosamine. Abnormal keratan sulfate refers to keratan sulfate that contains no sulfate or is improperly sulfated, and therefore does not contain the sulfated carbon 6 of N-acetylglucosamine present in normal keratan sulfate. Normal and abnormal keratan sulfate can be distinguished using any of a variety methods known in the art, for example, immunoreactivity using antibodies that specifically bind normal keratan sulfate such as antibody 5D4, hexosamine sugar analysis, sensitivity to keratanase digestion, and the like, as taught herein and in publications such as Nakazawa et al., J. Biol. Chem. 259:13751-13757 (1984) and Yang et al., Am. J. Ophthalmol. 106:65-71 (1988).

The present invention also provides active fragments of a corneal GlcNAc6ST polypeptide. As used herein, the term “active fragment” means a polypeptide fragment that has substantially the amino acid sequence of a portion of a corneal GlcNAc6ST and that catalyzes the sulfation of keratan sulfate. An active fragment of a corneal GlcNAc6ST can have, for example, substantially the amino acid sequence of a portion of human GlcNAc6ST (SEQ ID NO: 2), which can be, for example, the catalytic domain. In one embodiment, an active fragment contains substantially the sequence of Ser²⁷ to Asn³⁹⁵. Sulfotransferase activity can be assayed using methods known in the art such as those used in Example VI or those used in Habuchi et al., Glycobioloqy 6:51-57 (1996).

In one embodiment, a polypeptide of the invention has substantially the amino acid sequence of human corneal GlcNAc6ST (SEQ ID NO:2). As used herein, the term “substantially the amino acid sequence” when used in reference to a corneal GlcNAc6ST polypeptide or an active fragment thereof, is intended to mean an identical sequence, or a similar, non-identical sequence that is considered by those skilled in the art to be a functionally equivalent amino acid sequence. For example, an amino acid sequence that has substantially the amino acid sequence of a human GlcNAc6ST polypeptide (SEQ ID NO: 2) can have one or more modifications such as amino acid additions, deletions or substitutions relative to the amino acid sequence of SEQ ID NO:2, provided that the modified polypeptide retains substantially the ability to catalyze the sulfation of keratan sulfate.

The present invention also provides substantially purified antibody material that specifically binds a corneal GlcNAc6ST that catalyzes the sulfation of keratan sulfate. Such antibody material, which can be polyclonal or monoclonal antibody material, specifically binds a corneal GlcNAc6ST such as human GlcNAc6ST having the amino acid sequence SEQ ID NO: 2.

A corneal GlcNAc6ST polypeptide or polypeptide fragment can be used to prepare the substantially purified antibody material of the invention. Such antibody material can be, for example, substantially purified polyclonal antiserum or monoclonal antibody material. The antibody material of the invention can be useful, for example, in determining the level of expression of corneal GlcNAc6ST in a subject.

As used herein, the term “antibody material” is used in its broadest sense to include polyclonal and monoclonal antibodies, as well as polypeptide fragments of antibodies that retain a specific binding activity for a corneal GlcNAc6ST polypeptide of at least about 1×10⁵ M⁻¹. One skilled in the art would know that anti-corneal GlcNAc6ST antibody fragments such as Fab, F(ab′)₂ and Fv fragments can retain specific binding activity for a corneal GlcNAc6ST polypeptide and, thus, are included within the definition of antibody material. In addition, the term “antibody material,” as used herein, encompasses non-naturally occurring antibodies and fragments containing, at a minimum, one V_{H} and one V_{L} domain, such as chimeric antibodies, humanized antibodies and single chain Fv fragments (scFv) that specifically bind a corneal GlcNAc6ST polypeptide. Such non-naturally occurring antibodies can be constructed using solid phase peptide synthesis, produced recombinantly or obtained, for example, by screening combinatorial libraries consisting of variable heavy chains and variable light chains as described by Borrebaeck (Ed.), Antibody Engineering (Second edition) New York: Oxford University Press (1995)).

Antibody material “specific for” a corneal GlcNAc6ST polypeptide, or that “specifically binds” a corneal GlcNAc6ST polypeptide, binds with substantially higher affinity to human corneal GlcNAc6ST (SEQ ID NO:2) or another corneal GlcNAc6ST than to other sulfotransferases. The substantially purified antibody material of the invention also can bind with significantly higher affinity to a GlcNAc6ST that catalyzes the sulfation of keratan sulfate than to another sulfotransferase that does not catalyze the sulfation of keratan sulfate, such as human intestinal GlcNAc6ST (SEQ ID NO: 4).

Anti-corneal GlcNAc6ST antibody material can be prepared, for example, using a human GlcNAc6ST fusion protein or a synthetic peptide encoding a portion of a corneal GlcNAc6ST polypeptide such as SEQ ID NO:2 as an immunogen. One skilled in the art would know that a purified corneal GlcNAc6ST polypeptide, which can be produced recombinantly, or a fragment of a corneal GlcNAc6ST, including a peptide portion of a corneal GlcNAc6ST such as a synthetic peptide, can be used as an immunogen. Non-immunogenic fragments or synthetic peptides of a corneal GlcNAc6ST can be made immunogenic by coupling the hapten to a carrier molecule such as bovine serum albumin (BSA) or hemocyanin from horseshoe crab or keyhole limpet. In addition, various other carrier molecules and methods for coupling a hapten to a carrier molecule are well known in the art as described, for example, by Harlow and Lane, Antibodies: A Laboratory Manual (Cold Spring Harbor Laboratory Press, 1988)) and Ausubel et al., Current Protocols in Molecular Biology John Wiley & Sons, Inc. New York (2000).

The term “substantially purified,” as used herein in reference to antibody material, means that the antibody material is substantially devoid of polypeptides, nucleic acids and other cellular material with which an antibody is normally associated in a cell. The claimed antibody material that specifically binds a corneal GlcNAc6ST further is substantially devoid of antibody material of unrelated specificities, i.e. that does not specifically bind a corneal GlcNAc6ST. The antibody material of the invention can be prepared in substantially purified form using, for example, GlcNAc6ST affinity purification of polyclonal anti-corneal GlcNAc6ST antisera, by screening phage displayed antibodies against a corneal GlcNAc6ST polypeptide such as SEQ ID NO: 2, or as monoclonal antibodies prepared from hybridomas.

The present invention also provides a method of modifying an acceptor molecule by contacting the acceptor molecule with an isolated corneal GlcNAc6ST, or an active fragment thereof, under conditions that allow addition of a sulfate to a GlcNAc acceptor molecule, where the corneal GlcNAc6ST or active fragment thereof catalyzes the sulfation of keratan sulfate. In one embodiment, the acceptor molecule is modified to produce sulfated keratan sulfate immunoreactive with antibody 5D4. A corneal GlcNAc6ST useful for modifying an acceptor molecule according to a method of the invention can have, for example, substantially the amino acid sequence of human GlcNAc6ST (SEQ ID NO: 2) or an active fragment thereof.

The term “acceptor molecule,” as used herein, means a molecule that is acted upon, or “modified,” by a protein having sulfotransferase activity. Thus, an acceptor molecule is a molecule that accepts the transfer of a sulfate. An acceptor molecule can be in substantially pure form or in an impure form such as in a host cell or cellular extract, and, furthermore, can be a naturally occurring molecule or a completely or partially synthesized molecule. One skilled in the art understands that an acceptor molecule can contain one or more sugar residues prior to modification and can be further modified, if desired, to contain additional sugar residues. An acceptor molecule useful in the invention can contain, for example, the keratan sulfate core structure (Galβ1→4GalNAc→R). An exemplary acceptor molecule is keratan sulfate I.

The present invention further provides an isolated nucleic acid molecule which encodes a corneal GlcNAc6ST or an active fragment thereof that catalyzes the sulfation of keratan sulfate. An isolated nucleic acid molecule of the invention can encode, for example, a corneal GlcNAc6ST that has substantially the amino acid sequence of human corneal GlcNAc6ST (SEQ ID NO: 2) and can be, for example, SEQ ID NO: 1 or a portion thereof. The sulfated keratan sulfate formed by the catalytic activity of the encoded corneal GlcNAc6ST can be immunoreactive with the antibody 5D4 or can be hydrolyzable by keratanase. In one embodiment, a nucleic acid molecule of the invention encodes an active fragment that has substantially the amino acid sequence of a portion of a corneal GlcNAc6ST and that catalyzes the sulfation of keratan sulfate, provided that the fragment is not EST AI814200 or a segment thereof. The invention further provides vectors and related host cells that contain a nucleic acid molecule encoding a corneal GlcNAc6ST or active fragment thereof that catalyzes the sulfation of keratan sulfate. In one embodiment, such a vector is a mammalian expression vector.

As used herein, the term “nucleic acid molecule” means any polymer of two or more nucleotides, which are linked by a covalent bond such as a phosphodiester bond, a thioester bond, or any of various other bonds known in the art as useful and effective for linking nucleotides. Such nucleic acid molecules can be linear, circular or supercoiled, and can be single stranded or double stranded. Such molecules can be, for example, DNA or RNA, or a DNA/RNA hybrid.

A sense or antisense nucleic acid molecule or oligonucleotide of the invention also can contain one or more nucleic acid analogs. Nucleoside analogs or phosphothioate bonds protect against degradation by nucleases are particularly useful in a nucleic acid molecule or oligonucleotide of the invention. A ribonucleotide containing a 2-methyl group, instead of the normal hydroxyl group, bonded to the 2′-carbon atom of ribose residues, is an example of a non-naturally occurring RNA molecule that is resistant to enzymatic and chemical degradation. Other examples of non-naturally occurring organic molecules include RNA containing 2′-aminopyrimidines, such RNA being 1000× more stable in human serum as compared to naturally occurring RNA (see Lin et al., Nucl. Acids Res. 22:5229-5234 (1994); and Jellinek et al., Biochemistry 34:11363-11372 (1995)).

Additional nucleotide analogs also are well known in the art. For example, RNA molecules containing 2′-O-methylpurine substitutions on the ribose residues and short phosphorothioate caps at the 3′- and 5′-ends exhibit enhanced resistance to nucleases (Green et al., Chem. Biol. 2:683-695 (1995)). Similarly, RNA containing 2′-amino-2′-deoxypyrimidines or 2′-fluro-2′-deoxypyrimidines is less susceptible to nuclease activity (Pagratis et al., Nature Biotechnol. 15:68-73 (1997)). Furthermore, L-RNA, which is a stereoisomer of naturally occurring D-RNA, is resistant to nuclease activity (Nolte et al., Nature Biotechnol. 14:1116-1119 (1996)); Klobmann et al., Nature Biotechnol. 14:1112-1115 (1996)). Such RNA molecules and methods of producing them are well known and routine in the art (see Eaton and Piekern, Ann. Rev. Biochem. 64:837-863 (1995)). DNA molecules containing phosphorothioate linked oligodeoxynucleotides are nuclease resistant (Reed et al., Cancer Res. 50:6565-6570 (1990)). Phosphorothioate-3′ hydroxypropylamine modification of the phosphodiester bond also reduces the susceptibility of a DNA molecule to nuclease degradation (see Tam et al., Nucl. Acids Res. 22:977-986 (1994)). Furthermore, thymidine can be replaced with 5-(1-pentynyl)-2′-deoxoridine (Latham et al., Nucl. Acids Res. 22:2817-2822 (1994)). It is understood that nucleic acid molecules, including antisense molecules and oligonucleotides, containing one or more nucleotide analogs or modified linkages are encompassed by the invention.

The invention also provides vectors which contain a nucleic acid molecule encoding a corneal GlcNAc6ST or active fragment thereof which catalyzes sulfation of keratan sulfate. Such vectors, which can be cloning vectors or expression vectors, provide a means to transfer an exogenous nucleic acid molecule into a prokaryotic or eukaryotic host cell. Contemplated vectors include those derived from a virus, such as a bacteriophage, a baculovirus or a retrovirus, and vectors derived from bacteria or a combination of bacterial and viral sequences, such as a cosmid or a plasmid. The vectors of the invention can advantageously be used to clone or express a corneal GlcNAc6ST or an active fragment thereof. Various vectors and methods for introducing such vectors into a host cell are described, for example, in Ausubel et al., supra, 2000.

In addition to containing a nucleic acid molecule encoding a corneal GlcNAc6ST or active fragment thereof, a vector of the invention also can contain, if desired, one or more of the following elements: an oligonucleotide encoding, for example, a termination codon or a transcription or translation regulatory element; one or more selectable marker genes, such as an ampicillin, tetracycline, neomycin, hygromycin or zeomycin resistance gene, which is useful for selecting stable transfectants in mammalian cells; one or more enhancer or promoter sequences, which can be obtained, for example, from a viral, bacterial or mammalian gene; transcription termination and RNA processing signals, which are obtained from a gene or a virus such as SV40; an origin of replication such as an SV40, polyoma or E. coli origin of replication; versatile multiple cloning sites; and one or more RNA promoters such as a T7 or SP6 promoter, which allow for in vitro transcription of sense and antisense RNA.

In one embodiment, a vector of the invention is an expression vector. Expression vectors are well known in the art and provide a means to transfer and express an exogenous nucleic acid molecule in a host cell. Contemplated expression vectors include vectors that provide for expression in a host cell such as a bacterial cell, yeast cell, insect cell, frog cell, mammalian cell or other animal cell. Such expression vectors include regulatory elements specifically required for expression of the DNA in a cell, the elements being located relative to the nucleic acid molecule encoding the corneal GlcNAc6ST so as to permit expression thereof. The regulatory elements can be chosen to provide constitutive expression or, if desired, inducible or cell type-specific expression. Regulatory elements required for expression have been described above and include transcription and translation start sites and termination sites. Such sites permit binding, for example, of RNA polymerase and ribosome subunits. A bacterial expression vector can include, for example, an RNA transcription promoter such as the lac promoter, a Shine-Delgarno sequence and an initiator AUG codon in the proper frame to allow translation of an amino acid sequence.

Mammalian expression vectors can be particularly useful and can include, for example, a heterologous or homologous RNA transcription promoter for RNA polymerase binding, a polyadenylation signal located downstream of the coding sequence, an AUG start codon in the appropriate frame and a termination codon to direct detachment of a ribosome following translation of the transcribed mRNA. Commercially available mammalian expression vectors include pSI, which contains the SV40 enhancer/promoter (Promega; Madison, Wis.); pTarget™ and pCI, which each contain the cytomegalovirus (CMV) enhancer/promoter (Promega); pcDNA3.1, a CMV expression vector (Invitrogen; Carlsbad, Calif.); and pRc/RSV, which contains Rous sarcoma virus (RSV) enhancer/promoter sequences (Invitrogen). In addition to these constitutive mammalian expression vectors, inducible expression systems are available, including, for example, an ecdysone-inducible mammalian expression system such as pIND and pVgRXR from Invitrogen. These and other mammalian expression vectors are commercially available or can be assembled by those skilled in the art using well known methods. An example of a eukaryotic expression vector of the invention is -pcDNA3.1, described in Example VI below.

The invention also provides a host cell containing a vector that includes a nucleic acid molecule encoding a corneal GlcNAc6ST or an active fragment thereof. Such a host cell can be used to replicate the vector and, if desired, to express and isolate substantially pure recombinant corneal GlcNAc6ST using well known biochemical procedures (see Ausubel, supra, 2000). In addition, a host cell of the invention can be used in an in vitro or in vivo method to transfer sulfate to an acceptor molecule such as keratan sulfate.

Host cells expressing a corneal GlcNAc6ST or an active fragment thereof also can be used to screen for agents that increase the expression or activity of a corneal GlcNAc6ST or to screen for selective inhibitors of a corneal GlcNAc6ST of the invention. Agents that increase expression or activity of GlcNAc6ST can be administered to a subject to prevent or treat a condition resulting from a deficiency of sulfated keratan sulfate such as macular corneal dystrophy type I or type II.

Examples of host cells useful in the invention include bacterial, yeast, frog and mammalian cells. Various mammalian cells useful as host cells include, for example, mouse NIH/3T3 cells, CHO cells, COS cells and HeLa cells. In addition, mammalian cells obtained, for example, from a primary explant culture are useful as host cells. In one embodiment, the primary, explanted cells are corneal cells. Primary, explanted host cells such as corneal cells can be obtained from a subject for the purpose of introducing into these cells in vitro an expression vector as described above. Additional host cells include non-human mammalian embryonic stem cells, fertilized eggs and embryos, which can be routinely used to generate transgenic animals, such as mice, which express the novel corneal GlcNAc6ST of the invention. Transgenic mice expressing corneal GlcNAc6ST can be used, for example, to screen for compounds that enhance or inhibit the sulfotransferase expression or activity of this enzyme. Methods for introducing a vector into a host cell include electroporation, microinjection, calcium phosphate, DEAE-dextran and lipofection methods well known in the art (see, for example, Ausubel, supra, 2000).

Also provided herein is an oligonucleotide that contains a nucleotide sequence having at least 10 contiguous nucleotides of SEQ ID NO: 1 or 38, or a nucleotide sequence complementary thereto, provided that the oligonucleotide sequence does not consist of a sequence of GenBank accession number AI824100. An oligonucleotide of the invention can have, for example, at least 15 contiguous nucleotides of SEQ ID NO: 1 or 38 or a nucleotide sequence complementary thereto.

Oligonucleotides of the invention can advantageously be used, for example, as primers for PCR or sequencing, as probes for diagnostic and other assays, and in therapeutic methods. An oligonucleotide of the invention can incorporate, if desired, a detectable moiety such as a radiolabel, fluorochrome, luminescent tag, ferromagnetic substance, or a detectable agent such as biotin, and can be useful, for example, for detecting mRNA expression of a corneal GlcNAc6ST in a cell or tissue and for Southern analysis, for example, to detect large chromosomal deletions or rearrangements (see below). Those skilled in the art can determine the appropriate length of a corneal GlcNAc6ST oligonucleotide for a particular application. An oligonucleotide of the invention contains a nucleotide sequence having, for example, at least, 10, 12, 14, 16, 18, 20, 25, 30, 35, 40, 50, 100 or 200 contiguous nucleotides of SEQ ID NO: 1 or 38, or a nucleotide sequence complementary thereto.

The invention also provides an isolated antisense nucleic acid molecule which contains a nucleotide sequence that specifically binds to SEQ ID NO: 1 or 38. Such an isolated antisense nucleic acid molecule can have, for example, at least 20 nucleotides complementary to SEQ ID NO: 1 or 38. In one embodiment, an isolated antisense nucleic acid molecule has at least 20 nucleotides complementary to SEQ ID NO: 1 or SEQ ID NO: 38 and contains a nucleotide sequence complementary to the sequence ATG.

An antisense nucleic acid molecule of the invention specifically binds to the nucleotide sequence of SEQ ID NO:1 or 38. An antisense nucleic acid molecule that “specifically binds” SEQ ID NO: 1 or SEQ ID NO: 38, binds with substantially higher affinity to that particular nucleotide sequence than to an unrelated nucleotide sequence.

As disclosed herein, restriction fragment polymorphism and nucleotide sequence analysis have revealed mutations in the coding region of corneal GlcNAc6ST in patients with type I macular corneal dystrophy (FIG. 5 and Table 1). Furthermore, polymerase chain reaction experiments and Southern blotting analyses have shown that patients with type II macular corneal dystrophy have altered or deleted regions upstream of the gene encoding corneal GlcNAc6ST (FIG. 6 and Table 1). As further disclosed herein, when human corneal GlcNAc6ST and murine intestinal GlcNAc6ST were expressed in HeLa cells, sulfated keratan sulfate was produced, as determined the antibody 5D4, which is specific for sulfated keratan sulfate (see Table 2). These results indicate that increased expression of activity of a GlcNAc6ST such that sulfated keratan sulfate is produced can correct the biochemical deficiency that causes macular corneal dystrophy and can, therefore, be useful in preventing and treating this disorder.

Thus, the invention provides a method of treating a subject with macular corneal dystrophy by administering to the subject an effective amount of an agent that increases the expression or activity of a GlcNAc6ST, whereby the amount of sulfated keratan sulfate in the cornea of the subject is elevated. The macular corneal dystrophy to be treated according to a method of the invention can be, for example, macular corneal dystrophy type I or type II. In one embodiment, the expression or activity of endogenous GlcNAc6ST is elevated. In another embodiment, the expression or activity of GlcNAc6ST is elevated using a nucleic acid molecule which encodes a GlcNAc6ST or an active fragment thereof that catalyzes the sulfation of keratan sulfate. Such a GlcNAc6ST can be, for example, a corneal GlcNAc6ST, for example, a human corneal GlcNAc6ST having, substantially the amino acid sequence of human GlcNAc6ST (SEQ ID NO:2), or another corneal or noncorneal GlcNAc6ST that catalyzes the sulfation of keratan sulfate. In a further embodiment, the agent is a GlcNAc6ST polypeptide, or active fragment thereof, that catalyzes the sulfation of keratan sulfate. Such an GlcNAc6ST polypeptide can be, for example, a murine GlcNAc6ST, a human GlcNAc6ST or a corneal GlcNAc6ST and can have, for example, substantially the amino acid sequence of human corneal GlcNAc6ST (SEQ ID NO:2). In yet a further embodiment, an agent useful for treating a subject with macular corneal dystrophy according to a method of the invention increases the transcription of a GlcNAc6ST that catalyzes the sulfation of keratan sulfate, and can, for example, selectively increase transcription of a GlcNAc6ST in the cornea of the subject.

The invention also provides a method of treating a subject with macular corneal dystrophy by administering in vitro to primary, explanted corneal cells an effective amount of an agent that increases the expression or activity of a GlcNAc6ST. The cells are subsequently introduced into the cornea of the subject, whereby the amount of sulfated keratan sulfate in a subject is elevated.

As used herein, the term “macular corneal dystrophy” means a disease characterized by the progressive formation of punctate opacities in the cornea and by a partial or complete deficiency of sulfated keratan sulfate in the cornea. The term macular corneal dystrophy encompasses both type I and type II forms of the disease.

The term “subject,” as used herein, refers to any animal, preferably a mammal such as a human, having corneal tissue that normally contains sulfated keratan sulfate.

The term “agent that increases expression or activity of a GlcNAc6ST” means an agent, which when administered to a subject having defective or deficient GlcNAc6ST activity in the cornea or to corneal cells having defective or deficient GlcNAc6ST activity in the cornea, increases the sulfotransferase activity of a GlcNAc6ST polypeptide in comparison with an untreated subject or untreated cells, such that the amount of sulfated keratan sulfate in the cornea of the subject or in the corneal cells is elevated. It is understood that the term “increased,” as used herein, encompasses wild-type or higher levels of expression of activity of a GlcNAc6ST that catalyzes the sulfation of keratan sulfate, as well as protein expression or activity that is enhanced relative to expression or activity in an untreated subject but falls below wild-type levels.

As set forth above, a N-acetylglucosamine-6-sulfotransferase or GlcNAc6ST useful in the invention is an enzyme that catalyzes the addition of a sulfate ester to keratan sulfate when expressed in cornea and, preferably, is an enzyme that catalyzes the addition of a sulfate ester to carbon 6 of N-acetylglucosamine of keratan sulfate I in cornea. For example, both human corneal GlcNAc6ST (SEQ ID NO: 2) and murine intestinal GlcNAc6ST (SEQ ID NO: 5) are GlcNAc6STs as defined herein, since these proteins, when transfected in HeLa cells, produce sulfated keratan sulfate detectable with 5D4 antibody. As further set forth above, a GlcNAc6ST useful in a method of the invention generally has at least 50% amino acid sequence identity to human corneal GlcNAc6ST (SEQ ID NO:2), and can have 55%, 60%, 65%, 70%, 75%, 80% or more % sequence identity to human GlcNAc6ST (SEQ ID NO:2). “Corneal” GlcNAc6STs are a subset of GlcNAc6STs, which have at least 90% amino acid identity with human corneal GlcNAc6ST (SEQ ID NO: 2).

A variety of means can be used to administer an agent according to a method of the invention. In a method of treating a subject with macular corneal dystrophy, an agent can be administered, for example, intravenously or intramuscularly or by ballistic gun; microinjection; electroporation; ingestion; inhalation; absorption such as absorption through the skin, cornea or tear duct; or by any other method of administration known in the art. In one embodiment, an agent is administered by injection into the cornea of a subject. One skilled in the art understands that a preferred method of administration depends, in part, on the type of agent to be administered.

An agent that increases GlcNAc6ST expression or activity is administered to a subject in an effective amount. The term “effective amount,” as used herein in regard to an agent that increases expression or activity of a GlcNAc6ST, means an amount of the agent that elevates the amount of sulfated keratan sulfate in the cornea of the subject, and preferably results in an amount of sulfated keratan sulfate that reduces or prevents formation of opacities in the cornea. An increase in sulfated keratan sulfate can be measured by one of a variety of routine assays known to one of skill in the art as disclosed herein in Examples III and VI. Such assays include histochemical staining using antibody specific for sulfated keratan sulfate, hexosamine sugar analysis, sensitivity to keratanase digestion, and the like. Reduced formation of opacities in cornea can be determined using methods available to those of skill in the art, such as light and electron microscopic analyses.

An effective amount of an agent to be used in the methods of the invention depends, in part, on the chemical and biological properties of the agent and the method of administration. Exemplary concentration ranges useful in the invention include 10 μg/ml to 500 mg/ml, 100 μg/ml to 250 mg/ml, and 1 mg/ml to 200 mg/ml.

In a method of the invention for treating macular corneal dystrophy, an effective amount of the agent can be administered as a single dose or as multiple doses. Multiple doses can be administered using a regular, periodic dose schedule such as one administration per day, or weekly or monthly. Symptomatic administration, where an effective amount is administered upon clinical determination of disease progression or upon experiencing deterioration of vision, also can be useful in a method of the invention.

A method of the invention can be practiced with one of a variety of agents that increase expression or activity of a GlcNAc6ST, which can be a corneal GlcNAc6ST such as human corneal GlcNAc6ST or another, non-corneal GlcNAc6ST. As used herein, the term “agent” means an inorganic or organic molecule such as a drug; a peptide, or a variant or modified peptide or a peptide-like molecule such as a peptidomimetic or peptoid; or a polypeptide such as a GlcNAc6ST, or an active fragment of a GlcNAc6ST; an antibody or active fragment thereof such as an Fv, Fd or Fab fragment or another fragment that contains a binding domain; a nucleic acid molecule which can encode, for example, a GlcNAc6ST such as human corneal GlcNAc6ST, and can be incorporated, if desired, into a vector such as one of the plasmid, phage or other vectors described herein; or a cell into which has been introduced a vector for expressing a polypeptide such as a GlcNAc6ST or an active fragment thereof. Exemplary agents include a co-factor or a sulfate-donating compound that increases the sulfotransferase activity of a mutant GlcNAc6ST variant such as a variant of human corneal GlcNAc6ST (SEQ ID NO:2), murine GlcNAc6ST (SEQ ID NO:5) or another mammalian or primate corneal GlcNAc6ST; or a vector containing a nucleic acid encoding a GlcNAc6ST; a transcription factor that binds the mutated upstream region of the CHST6 gene and increases GlcNAc6ST expression, a transcription factor that increases expression of weakly active mutant GlcNAc6ST; or a vector containing a transcription factor that increases GlcNAc6ST expression.

If desired, an agent can be combined with, or dissolved in, an acceptable carrier, which can facilitate uptake of the agent by the subject. Such a carrier can be, for example, DMSO or ethanol, or an aqueous solvent such as water or a buffered aqueous solution. Other acceptable carriers include standard pharmaceutical carriers, such as phosphate buffered saline solution, water and emulsions such as an oil/water or water/oil emulsion, and various types of wetting agents.

In one embodiment of the invention, an agent that increases the expression or activity of GlcNAc6ST is a nucleic acid molecule. A nucleic acid molecule can encode a polypeptide that increases the expression or activity of a GlcNAc6ST. For example, a nucleic acid molecule encoding a GlcNAc6ST polypeptide will, when expressed, increase the expression of GlcNAc6ST. An exemplary polypeptide is a GlcNAc6ST that is naturally expressed in cornea, such as the GlcNAc6ST of SEQ ID NO:2 or murine GlcNAc6ST (SEQ ID NO: 5). One of skill in the art will recognize that the high degree of homology between the human and murine GlcNAc6ST expressed in cornea demonstrates that GlcNAc6ST from a variety of mammals also can be useful in the methods of the invention. A mammalian GlcNAc6ST useful in the invention can be identified by routine methods, for example, by preparing a corneal cDNA library from a mammal of interest, hybridizing with a probe such as an oligonucleotide that specifically binds a GlcNAc6ST; and amplifying a GlcNAc6ST-encoding cDNA.

In one embodiment, the expression or activity of endogenous GlcNAc6ST is elevated. As used herein, an “endogenous” GlcNAc6ST means a GlcNAc6ST polypeptide that is expressed from a gene natively present in the subject.

An agent useful in the invention can be a nucleic acid molecule that increases the transcription of GlcNAc6ST. For example, a nucleic acid molecule useful in the invention can encode a transcription factor that increases the transcription of GlcNAc6ST. As another example, a nucleic acid molecule can contain an upstream regulatory sequence that, when present in a cell, increases transcription of GlcNAc6ST, for example, by competing for a factor that normally inhibits the endogenous GlcNAc6ST.

An agent useful for increasing the expression or activity of a GlcNAc6ST can be identified using routine methods. For example, a cell that normally produces a control (low) level sulfated keratan sulfate, such as a HeLa cell, can be contacted with a candidate agent, and the amount of sulfated keratan sulfate assayed, for example, by immunoreactivity with the 5D4 antibody (see Example VI). A candidate agent that elevates the amount of sulfated keratan sulfate in the HeLa cell is an agent that increases expression or activity of a GlcNAc6ST useful for treating macular corneal dystrophy type I or type II.

Treatment of a subject with macular corneal dystrophy by administration of a nucleic acid molecule can be carried out using the above-described methods of administration, and can be accompanied by a compound that facilitates transfection into cells, including calcium phosphate, DEAE dextran, cationic lipids, liposomes, polylysine, or the like. Further, a nucleic acid molecule can be administered, if desired, in a viral vector, which can facilitate transfection into cells and can also improve tissue specificity, reduce death of transformed cells, and the like. These and additional methods of administering a nucleic acid molecule to a subject are known in the art as described, for example, in Chang (Ed.), Somatic Gene Therapy CRC Press, Inc. (1994).

Viral vectors that can be used in the administration of a nucleic acid molecule that increases expression or activity of a GlcNAc6ST into a subject or corneal cell. Such viral vectors include, for example, Herpes simplex virus vectors, vaccinia virus vectors, cytomegalovirus vectors, Moloney murine leukemia virus vectors, adenovirus vectors, lentivirus vectors, adeno-associated virus vectors, retrovirus vectors, and the like. Especially preferred viral vectors are adenovirus and retroviral vectors.

In one embodiment, a nucleic acid molecule is administered to the cornea of the subject. Administration of a nucleic acid molecule to cornea can be carried out using one of numerous methods well known in the art of gene therapy, including ballistic gun delivery, lentiviral transformation, adenoviral transformation, cytomegaloviral transformation, microinjection and electroporation as described further below.

Ballistic gun delivery can be useful in the methods of the invention and can be performed, for example, as described in Tanelian et al., BioTechniques, 23:484-488 (1997), to achieve focal delivery and expression of a plasmid in corneal epithelium with high efficiency. In this method, 0.2-0.5 mg gold particles are coated with plasmid DNA, which is then delivered into cornea using a ballistic gun. The depth of delivery of the plasmid DNA is a function of the pressure of the gun, thus facilitating delivery of plasmid DNA to a desired depth in cornea.

Lentivirus also can be useful to administer a nucleic acid molecule in the methods of the invention. Cells can be transduced with lentivirus in vitro or in situ as described, for example, in Wang et al., Gene Therapy 7:196-200 (2000). Corneal endothelial cells, epithelial cells and stromal keratocytes in human cornea obtained after penetrating keratoplasty can be exposed to lentivirus encoding a nucleic acid molecule useful in the invention. Exposed cells can continue to express the encoded protein for at least 60 days after transduction.

Adenovirus has can be used to deliver a nucleic acid molecule to cornea in a method of the invention, for example, as described in Larkin et al., Transplantation 61:363-370 (1996), for expression of the encoded polypeptide such as a corneal GlcNAc6ST in endothelial cells. Adenovirus also can be used to administer a nucleic acid molecule to cornea in vivo after surgical removal of superficial epithelial cells from the cornea (U.S. Pat. No. 5,827,702).

Microinjection and electric pulse also can be used in the methods of the invention introduce cytomegalovirus and the plasmid expression vector pCH110 into cornea (Sakamoto et al., Hum. Gene Ther. 10:2551-2557 (1999), and Oshima et al., Gene Therapy 5:1347-1354 (1998)). Injection of virus or plasmid into the anterior chamber at the limbus, followed by electric pulses results in transduction of corneal endothelial cells.

In another embodiment of the invention, an agent that increases the expression or activity of GlcNAc6ST is administered to cells in vitro. Exemplary cells to which the agent can be administered include autographic or allographic stem cells, primary explanted corneal cells, allographic or xenographic corneal cells, as well as other cells that can be transplanted into cornea. In accordance with this embodiment, subsequent to administering the agent to the cells, the cells are introduced into the cornea of the subject to be treated. Alternatively, the cells can be in the form of a cornea graft, in which case the corneal graft is placed onto the eye of the subject subsequent to administering the agent to the corneal graft. Methods for placing the cells or corneal graft into the cornea or onto the eye of the subject are known in the art and include microinjection and established keratoplasty techniques.

In accordance with another embodiment of the invention, a method of treating a patient with macular corneal dystrophy can be carried out by administering a polypeptide as an agent that increases expression or activity of GlcNAc6ST. Such a polypeptide can be, for example, a transcription factor that increases the expression of GlcNAc6ST, or a GlcNAc6ST or active fragment thereof, where the GlcNAc6ST catalyzes the sulfation of keratan sulfate. An exemplary polypeptide is a GlcNAc6ST that is naturally expressed in cornea, such as the human corneal GlcNAc6ST of SEQ ID NO:2 or murine GlcNAc6ST (SEQ ID NO: 5). One of skill in the art will recognize that the high degree of homology between the human and murine GlcNAc6ST expressed in cornea demonstrates that a variety of mammalian GlcNAc6STs can be used in the methods of the invention.

It is understood that a method of the invention also can be used to prophylactically treat an individual susceptible to MCD type I or type II, but who has no symptoms of opacities, loss of vision or “ground-glass” appearance of the cornea. Such an individual may have, for example, one or more family members with MCD type I or type II and may therefore be at high risk of developing MCD in the future. Such an individual may be determined to have mutations in one or both copies of the corneal GlcNAc6ST gene, CHST6, which may be known or suspected to result in decreased expression or activity of corneal GlcNAc6ST.

A method of the invention can also be used to prevent other conditions characterized by a deficiency of normal sulfated keratan sulfate. One of skill in the art will recognize that patients with mutations that lower the expression or activity of GlcNAc6ST in tissues such as cartilage in addition to cornea, for example, MCD type I patients, may develop a condition arising from absent or lowered amounts of sulfated keratan sulfate in cartilage or serum, for example, arthritis.

The results disclosed herein also provide the basis for a method of monitoring therapeutic efficacy in a subject being treated for macular corneal dystrophy. The method includes the steps of obtaining a test sample from the subject, determining a sample level of expression or activity of GlcNAc6ST in the test sample and comparing the sample level to a reference level from the subject, where an increase in the sample level relative to the reference level is indicative of productive therapy.

As used in the context of a course of therapy, “productive therapy” refers to the ability of the therapy to prevent, decrease or stop progression of opacities in cornea. Such a method has particular utility when the pre-therapeutic level of GlcNAc6ST expression in the cornea of a subject is below that of an individual that does not have macular corneal dystrophy. Comparison of the sample level to a reference level from the subject thereby serves to indicate whether the therapy is efficacious or not.

A sample level of GlcNAc6ST expression can be determined using one of a variety of types of samples, such as a serum sample, a cartilage sample or a corneal sample. The level of GlcNAc6ST expression can be determined, for example, by measuring the amount of GlcNAc6ST-encoding mRNA or GlcNAc6ST polypeptide present in the sample. Methods for measuring RNA or polypeptide expression are well known in the art, and include, for example, measuring the GlcNAc6ST mRNA level using a nucleic acid molecule that specifically hybridizes to a nucleotide sequence such as SEQ ID NO:1 or SEQ ID NO: 38, and measuring the GlcNAc6ST polypeptide level using an antibody that specifically binds GlcNAc6ST. The methods of the invention also can be practiced by determining the level of GlcNAc6ST activity in a sample, which is carried out by measuring the formation of product, sulfated keratan sulfate. Sulfated keratan sulfate can be measured using one of a variety of methods known in the art such as an immunoassay using an antibody specific for sulfated keratan sulfate such as 5D4, hexosamine sugar analysis, sensitivity to keratanase digestion, and the like. Additionally, the activity of GlcNAc6ST can be determined using a known detectable sulfate donor analog or a known detectable sulfate acceptor analog, where GlcNAc6-sulfotransferase activity is determined using a known method such as HPLC, absorption spectroscopy, fluorescence spectroscopy, or the like. Donor and acceptor analogs are known in the art and are commercially available from sources such as Sigma (St. Louis, Mo.).

In a method of the invention for monitoring therapeutic efficacy, a sample level is compared to a reference level from the same subject. As used herein, a “reference level” means a level of expression or activity of GlcNAc6ST obtained using the same assay used to obtain the “sample level” in a sample obtained from the same subject at an earlier time point than the test sample. Such a reference level of GlcNAc6ST expression or activity can be, for example, the pre-therapeutic level in the subject undergoing therapy, or a level at an earlier stage of therapy. It is understood that, preferably, the reference level of expression or activity is determined using the same or similar assay as used to analyze the test sample and that, for example, a test RNA level is compared to a reference RNA level, a test protein level is compared to a reference protein level, and a test level of GlcNAc6ST activity is compared to a reference level of GlcNAc6ST activity.

In another embodiment, a reference level can be determined as a function of the level observed in normal or unaffected individuals. Specifically, normal individuals expressing two copies of active C-GlcNAc6ST will have a certain level of activity; a reference level can be a level at least 50% of that of normal individuals, thus corresponding to a level of an individual expressing one copy of active C-GlcNAc6ST.

A method of the invention for monitoring therapeutic efficacy can be particularly useful when combined with a method of treating a subject with macular corneal dystrophy by administering an effective amount of an agent that increases the expression or activity of a GlcNAc6ST. Specifically, therapeutic efficacy of the agent can be monitored by obtaining a test sample from the subject, determining a sample level of GlcNAc6ST expression or activity in the test sample, and comparing this level to a reference level. Therapeutic efficacy is monitored on one or more occasions as desired.

The present invention also provides a method for diagnosing macular corneal dystrophy in an individual. The method includes the steps of determining a level of GlcNAc6ST expression or activity in a test sample from the individual and comparing the sample level to a control level, where a sample level significantly lower than the control level is diagnostic of macular corneal dystrophy. In one embodiment, a sample level is determined using a nucleic acid that specifically hybridizes to a nucleotide sequence such as SEQ ID NO: 1 or SEQ ID NO: 38. In another embodiment, a sample level is determined using an antibody that specifically binds GlcNAc6ST. One skilled in the art understands that a method of the invention for diagnosing macular corneal dystrophy can be used alone or can be used in conjunction with other methods of diagnosing or determining susceptibility to macular corneal dystrophy.

As referred to herein in regard to diagnosing macular corneal dystrophy, a “control level” means a level of GlcNAc6ST RNA or protein or a level of GlcNAc6ST activity in an unaffected individual from a family in which there is no past or present history of macular corneal dystrophy. It is understood that a control level can be a range of the values found in a population of unaffected individuals. One of skill in the art will recognize that the appropriate control level corresponds to the same species as the individual to be diagnosed and is of the same sample type, assayed under the same conditions.

The present invention also provides genetic methods of determining susceptibility to macular corneal dystrophy in an individual. These genetic methods are practiced by determining the presence or absence in an individual of a MCD-associated allele linked to a corneal N-acetylglucosamine-6-sulfotransferase (GlcNAc6ST) locus, where the presence of the MCD-associated allele indicates susceptibility to MCD in the individual. Such a method can be useful, for example, for determining susceptibility to macular corneal dystrophy type I or type II. In one embodiment, the MCD-associated allele is located within a corneal GlcNAc6ST gene, for example, within a corneal GlcNAc6ST coding region such as within the region coding the 3′-phosphate binding domain of corneal GlcNAc6ST. Such a MCD-associated allele can be, for example, a deletion, insertion or substitution in a GlcNAc6ST coding region. In another embodiment, the MCD-associated allele is within a corneal GlcNAc6ST 5′ regulatory region, and can be, for example, a replacement of a 5′ region of CHST6 with a 5′ region of CHST5 or a deletion of a 5′ region of CHST6.

As used herein, the term “corneal N-acetyglucosamine-6-sulfotrasferase locus” is synonymous with “corneal GlcNAc6ST locus” and means the chromosomal segment encoding a corneal GlcNAc6ST as defined hereinabove. In a human, the corneal GlcNAc6ST locus is CHST6.

A method of the invention for determining susceptibility to macular corneal dystrophy relies on a MCD-associated allele. As used herein, the term “MCD-associated allele” means a stably heritable molecular variation that tends to be inherited together with macular corneal dystrophy more often than would be expected according to traditional Mendelian genetics. A MCD-associated allele can be, for example, an allele linked to but outside of a corneal GlcNAc6ST gene, or can be within a corneal GlcNAc6ST gene itself, such as an allele in an upstream or downstream regulatory region, or an allele within a corneal GlcNAc6ST coding sequence (see Examples).

A MCD-associated allele useful in a method of the invention can be, for example, an insertion, deletion, rearrangement, single nucleotide polymorphism (snp), a microsatellite (ms) or a variable number tandem repeat (VNTR) polymorphism that tends to be inherited together macular corneal dystrophy type I or type II. A MCD-associated allele can be located in a coding or non-coding region of genomic DNA and may or may not affect corneal GlcNAc6ST expression or activity. When present in a coding region, an allele can be, for example, an insertion, deletion, missense mutation or frame-shift mutation. As disclosed herein in Example IV, nucleotide sequencing and PCR/restriction fragment length polymorphism analyses were used to identity several MCD-associated alleles. One MCD type I patient had a deletion of the entire coding region of CHST6; another patient had a 2-nucleotide insertion causing a frameshift at nucleotide 1106 of SEQ ID NO:1; and additional patients had a variety of missense mutations in the coding region of corneal GlcNAc6ST: 1213A→G, 1301C→A, 1512G→A, 1323C→T, and 840C→A. The missense mutation 1213A→G produces the amino acid substitution K174R; 1301C→A produces the amino acid substitution D203E; 1512G→A produces the amino acid substitution E274K; 1323C→T produces the amino acid substitution R211W; and 840C→A produces the amino acid substitution R50C. Expression in HeLa cells of corneal GlcNAc6ST variants containing the MCD-associated allele K174R, D203E, R211W or E274K resulted in little or no ability to catalyze sulfation of keratan sulfate, in contrast to expression of wild type corneal GlcNAc6ST (Table 2).

As further disclosed herein in Example V, a MCD-associated allele also can be located in a non-coding region such as a 5′ or 3′ regulatory region. Using polymerase chain reaction and Southern blot analyses, an altered 5′ regulatory sequence was detected in several MCD type II patients (see Example V). As disclosed herein, a MCD-associated allele that is associated with type II MCD can be, for example, an altered 5′ regulatory region having replaced sequence corresponding to the 5′ regulatory region of the proximal CHST5 gene (FIG. 6B and 6C). Other MCD type II patients had a large region deleted, where this region includes most of the CHST5 gene and the upstream region of CHST6 (FIG. 6D). In situ hybridization of corneal tissue from a MCD type II patient using a nucleotide specific for CHST6 showed no expression in corneal epithelial cells (FIG. 4). These results demonstrate that a MCD-associated allele which is associated with type II MCD can be present in a regulatory region of CHST6 and can reduce or prevent transcription of this gene.

A MCD-associated allele within a corneal GlcNAc6ST gene can result, for example, in production of a less active or inactive corneal GlcNAc6ST polypeptide or a reduced amount of a GlcNAc6ST polypeptide. A MCD-associated allele within a GlcNAc6ST gene can be located, for example, in an intron or in a 5′ or 3′ regulatory sequence and can influence the regulation of transcription or translation or splicing of a corneal GlcNAc6ST-encoding mRNA. Such an allele can, therefore, result in a change in corneal GlcNAc6ST gene expression level or expression of corneal GlcNAc6ST polypeptide variant. Where a MCD-associated allele is a nucleotide modification that results in one or more amino acid substitutions, deletions or insertions in a corneal GlcNAc6ST coding sequence and produces a variant corneal GlcNAc6ST polypeptide, such a variant may lack the ability to catalyze sulfation of keratan sulfate. For example, as disclosed herein in Example VI, a single amino acid substitution such as 50R→C, 174K→R, 203D→E, 211R→W, 217A→T and 274E→K results in a variant corneal GlcNAc6ST polypeptide that does not catalyze sulfation of keratan sulfate when expressed in HeLa cells (Table 2).

MCD is generally an autosomal recessive disorder, and, therefore, MCD will be correlated with the presence of a MCD-associated allele in both copies of the genomic DNA of an individual. As disclosed herein, affected individuals were either homozygous for a MCD-associated allele or were heterozygous for two different MCD-associated alleles (Table 1). In contrast, siblings of the affected individuals had only a single MCD-associated allele did not have symptoms of MCD (see, for example, FIG. 6C and 6D).

The presence or absence of a MCD-associated allele can be determined using one of a variety of molecular genotyping methods well known in the art. Such an allele can be detected, for example, by the genotyping methods disclosed herein in Examples IV and V, which disclose assays for determining the presence or absence of a MCD-associated allele such as DNA sequencing, restriction fragment length polymorphism analysis and Southern blot analysis. Additional assays that can be used to detect a MCD-associated allele include electrophoresis-based methods, allele-specific oligonucleotide hybridization, heteroduplex mobility assays, single strand conformational polymorphism analyses, denaturing gradient gel electrophoresis, cleavase fragment length polymorphism analyses and rolling circle amplification. One skilled in the art understands that sequence analysis and electrophoresis-based methods such as denaturing gradient gel electrophoresis or heteroduplex mobility assays are particularly useful for determining the presence or absence of a MCD-associated allele. See, in general, Birren et al. (Eds.) Genome Analysis: A Laboratory Manual Volume 1 (Analyzing DNA) New York, Cold Spring Harbor Laboratory Press (1997) and Ausubel et al., Current Protocols in Molecular Biology Chapter 2 (Supplement 49) John Wiley & Sons, Inc. New York (2000)).

Sequence analysis can be particularly useful for determining the presence or absence of a MCD-associated allele in a method of the invention. The term “sequence analysis,” as used herein in reference to one or more nucleic acids such as amplified fragments, refers to any manual or automated process by which the order of nucleotides in a nucleic acid is determined. It is understood that the term sequence analysis encompasses chemical (Maxam-Gilbert) and dideoxy enzymatic (Sanger) sequencing as well as variations thereof. Thus, the term sequence analysis includes capillary array DNA sequencing, which relies on capillary electrophoresis and laser-induced fluorescence detection and can be performed using, for example, the MegaBACE 1000 or ABI 3700. Sequence analysis also can be carried out using gel electrophoresis and detection methods such as fluorescence detection, radionuclide detection, and the like. Gel electrophoresis can be performed using, for example, the ABI 377 DNA sequencer. Also encompassed by the term sequence analysis are thermal cycle sequencing (Sears et al., Biotechniques 13:626-633 (1992)); solid-phase sequencing (Zimmerman et al., Methods Mol. Cell Biol. 3:39-42 (1992) and sequencing with mass spectrometry such as matrix-assisted laser desorption/ionization time-of-flight mass spectrometry MALDI-TOF MS (Fu et al., Nature Biotech. 16: 381-384 (1998)). The term sequence analysis also includes, for example, sequencing by hybridization (SBH), which relies on an array of all possible short oligonucleotides to identify a segment of sequences present in an unknown DNA (Chee et al., Science 274:61-614 (1996); Drmanac et al., Science 260:1649-1652 (1993); and Drmanac et al., Nature Biotech. 16:54-58 (1998)). One skilled in the art understands that these and additional variations are encompassed by the term sequence analysis as defined herein. See, in general, Ausubel et al., supra, 2000; Chapter 7.

The presence of a MCD-associated allele also can be determined using electrophoretic analysis. Electrophoresis, including gel or capillary electrophoresis, can be useful in separating amplified fragments containing alleles that differ in size. The term “electrophoretic analysis” or “electrophoresing,” as used herein in reference to one or more nucleic acids such as amplified fragments, means a process whereby charged molecules are moved through a stationary medium under the influence of an electric field. Electrophoretic migration separates nucleic acids primarily on the basis of their charge, which is in proportion to their size, with smaller nucleic acids migrating more quickly. The term electrophoretic analysis or electrophoresing includes analysis using both slab gel electrophoresis, such as agarose or polyacrylamide gel electrophoresis, and capillary electrophoresis. Capillary electrophoretic analysis, which generally occurs inside a small diameter (50-100 μm) quartz capillary in the presence of high (kilovolt level) separating voltages with separation times of a few minutes, can be particularly useful in a method of the invention. Using capillary electrophoretic analysis, nucleic acids such as amplified fragments are conveniently detected by UV absorption or fluorescent labeling, and single-base resolution can be obtained on fragments up to several hundred base pairs. Such methods of electrophoretic analysis, and variants thereof, are well known in the art as described, for example, in Ausubel et al., supra, 2000.

Cleavase fragment length polymorphism analysis also can be useful in the methods of the invention. Cleavase is an enzyme that cleaves junctions between single- and double-stranded regions of DNA. The gel filtration migration pattern of a DNA sample after cleavase digestion can be unique for each variant of the DNA sample according to the number of single- and double-stranded regions, the equilibrium between single- and double-stranded regions of the DNA, and the number of nucleotides in each cleavage fragment. This unique pattern, or “bar code” can be used to rapidly genotype a nucleic acid sample according to its migration pattern (see, for example, Tondella et al. J. Clin. Microbiol., 37:2402-2407 (1999); and U.S. Pat. Nos. 5,719,028 and 5,846,717).

Denaturing gradient gel electrophoresis (DGGE) also can be used to determine the presence or absence of a MCD-associated allele in a method of the invention. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

A heteroduplex mobility assay (HMA) is another well known assay that can be used to determine the presence or absence of a MCD-associated allele. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational polymorphism (SSCP) also can be used to determine the presence or absence of a MCD-associated allele in a method of the invention (see Hayashi, PCR Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Allele-specific oligonucleotide hybridization also can be used to determine the presence or absence of a MCD-associated allele in a method of the invention. Allele-specific oligonucleotide hybridization is based on the use of a labeled oligonucleotide probe having a sequence perfectly complementary, for example, to the nucleotides of a MCD-associated allele. Under appropriate conditions, the allele-specific probe hybridizes to a nucleic acid containing the MCD-associated allele but does not hybridize to one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a MCD-associated allele by using an allele-specific oligonucleotide primer that is perfectly complementary to the nucleotide sequence of the MCD-associated allele but which has one or more mismatches as compared to other alleles (Mullis et al. (Eds.), The Polymerase Chain Reaction, Birkhauser, Boston, (1994)). One skilled in the art understands that the one or more nucleotide mismatches that distinguish between the MCD-associated allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the MCD-associated and other alleles at the 3′ end of the primer.

Rolling circle amplification also can be used to determine the presence or absence of a MCD-associated allele in a method of the invention (Baner et al., Nucleic Acids Res. 26:5073-5078 (1998), and Lizardi et al., Nat. Genet. 19:225-232 (1998)). In rolling circle amplification, a linear probe is designed so that the 5′ and 3′ ends of the probe hybridize to immediately adjacent nucleotides in a specific nucleotide sequence. If the sample DNA has the specific sequence, the 5′ and 3′ ends are adjacent and the probe can be circularized using ligase. Sample DNA without the specific sequence will not result in the 5′ and 3′ ends hybridizing immediately adjacent one another, and therefore will not act as a successful template for circularization. The circularized probe can then be used in rolling circle replication to amplify the sequence prior to detection.

Other well-known approaches for determining the presence or absence of a MCD-associated allele include automated sequencing and RNAase mismatch techniques (Winter et al., Proc. Natl. Acad. Sci. 82:7575-7579 (1985)). Furthermore, one of skill in the art understands that amplification or cleavage methods described above also can be used in solid state methods, for example, using DNA microarrays for detection or as templates for enzymatic reactions (see Ausubel et al., supra, 2000). Similarly, mass spectroscopy can be used for the detection of cleavage or amplification products as described, for example, in U.S. Pat. Nos. 6,043,031, 5,605,798, and 5,547,835. It is understood that the methods of the invention can be practiced using these or other art-recognized assays for detecting polymorphic alleles.

In one embodiment, the invention provides a method of determining susceptibility to macular corneal dystrophy in an individual by determining the presence or absence of a MCD-associated allele linked to a GlcNAc6ST locus using enzymatic amplification of nucleic acid from the individual. In other embodiments, the presence or absence of a MCD-associated allele is determined by electrophoretic analysis, restriction fragment length polymorphism analysis, sequence analysis, or a combination of these techniques.

The following examples are intended to illustrate but not limit the present invention.

### EXAMPLE I

### Identification of Corneal GlcNAc6ST

This example describes identification and isolation of a nucleic acid sequence encoding a human corneal N-acetylglucosamine-6-sulfotransferase (C-GlcNAc6ST).

BLAST search of the GenBank EST database (Release 115.0) was carried out using the conserved regions of carbohydrate sulfotransferases corresponding to amino acids Ile¹⁴⁰ to Pro³⁰⁰ of HEC-GlcNAc6ST and Ala²²⁰ to Pro³⁰³ of GlcNAc6ST. Two candidate sequences were identified; the first, AI824100, is an EST derived from human lung squamous cell carcinoma, and the second is a recently reported human intestinal N-acetylglucosamine-6-sulfotransferase, also known as I-GlcNAc6ST or CHST5 (Lee et al., Biochem. Biophys. Res. Commun., 263:543-549 (1999)).

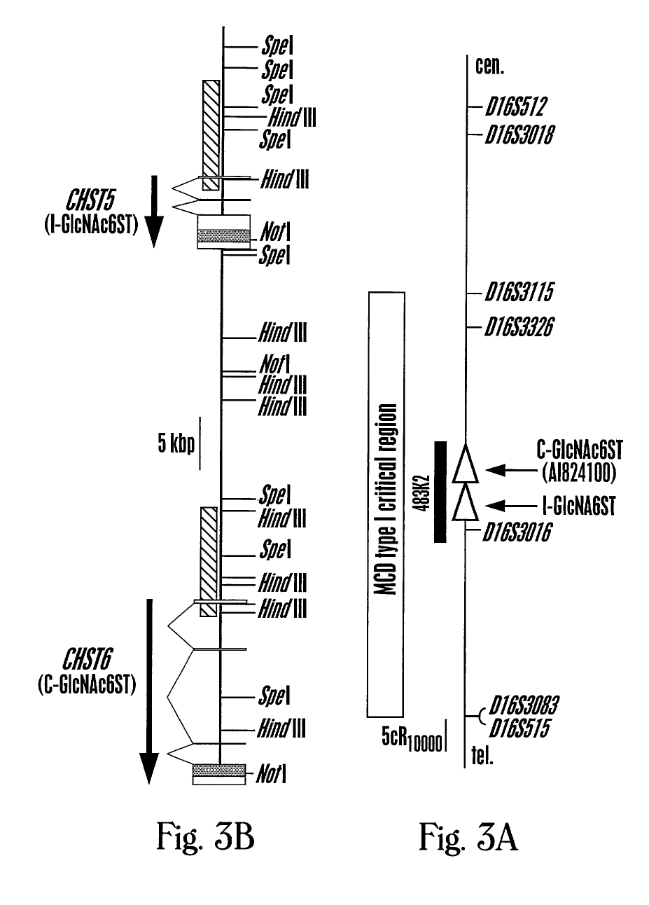

The Stanford G3 radiation hybrid panel (Research Genetics; Huntsville, Ala.) was used to map AI824100 and CHST5. PCR was carried out using primers ha115B6F (5′-AGAGCCGAAACCTGTCCGCC-3′; SEQ ID NO: 12) and ha115B6R (5′-GCGTAGAGTGCGCGGATCTCT-3′; SEQ ID NO: 13) to amplify CHST5, and CK71hF (5′-TATCTGCCTTGGCGCCGCAACCT-3′; SEQ ID NO: 14) and CK7lhR (5′-CCGTTGTCACGCGCCAGAGCCTT-3′; SEQ ID NO: 15) to amplify AI824100. PCR amplification was carried out in 10 μl reaction mixture containing 25 ng of hybrid cell DNA, 0.4 μM of each primer, 25 mM Tris-acetate pH 9.0, 50 mM potassium acetate, 1.25 mM magnesium acetate and 0.5 unit of polymerase mixture, which consists of 0.495 unit of AmpliTaq DNA polymerase (Perkin-Elmer; Foster City, Calif.) and 0.005 unit of Vent DNA polymerase (New England Biolabs; Beverly, Mass.). Amplification reactions were carried out by a PTC-100 Thermal Cycler (MJ Research; Watertown, Mass.) as follows: 2 minutes of denaturation at 96° C. prior to cycling; 35 cycles of denaturation at 96° C. for 30 seconds, annealing at 62° C. (for CHST5) and 65° C. (for AI824100) for 30 seconds, extension at 72° C. for 30 seconds; and a final extension at 72° C. for 5 minutes. Amplified DNA fragments were resolved by 0.7% agarose gel electrophoresis and detected on an UV transilluminator in the presence of ethidium bromide. Based on PCR amplification radiation hybrid scores, AI824100 and CHST5 were mapped between markers D16S3326 and D16S3016 on chromosome 16q22. Markers D16S3326 and D16S3016 lie between D16S3115 and D16S3083, a region previously shown to be linked MCD type I (FIG. 3A).

Full-length cDNA for AI824100 was isolated by 5′- and 3′-RACE reactions using human whole brain Marathon-ready cDNA (Clontech; Palo Alto, Calif.). Amplifications of the 5′- and 3′-regions were carried out according to the methods recommended by the manufacturer. The oligomers used as AI824100-specific primers for PCR were: CK71hR (SEQ IN NO: 15) for the first 5′-RACE, CK71h2R (5′-CGGGGAAAGGCACTGCAGGCGG-3′; SEQ ID NO: 16) for the second 5′-RACE, CK71hF for the first 3′-RACE, CK7lh2F (5′-CGACCCCGCGCTCAACCTACGCA-3′; SEQ ID NO: 17) for the second 3′-RACE. Amplified fragments were cloned into pBluescript II KS(+) (Stratagene; La Jolla, Calif.) and sequenced with an ABI377 DNA sequencer by using BigDye terminator kit (Perkin-Elmer).

### EXAMPLE II

### Characterization of Human Corneal GlcNAc6ST

This example shows that the corneal GlcNAc6ST identified herein is homologous to and proximally located to the intestinal GlNAc6ST, CHST5.

The full length cDNA human corneal GlcNAc6ST is predicted to encode a membrane protein consisting of 395 amino acids. Multiple sequence alignment of this cDNA was performed using Clustal W version 1.7 (Thompson et al., Nucleic Acids Res. 22:4673-4680 (1994)) and I-GlcNAc6ST (human intestinal GlcNAc-6-sulfotransferase; Lee et al., Biochem. Biophys. Res. Commun. 263:543-549 (1999)); HEC-GlcNAc6ST (human high-endothelial-cell GlcNAc-6-sulfotransferase; Bistrup et al., J. Cell Biol., 145:899-910 (1999)); GlcNAc6ST (human GlcNAc-6-sulfotransferase; Uchimura et al., J. Biochem., 124:670-678 (1998)); KSG6ST (human KS Gal-6-sulfotransferase; (Fukuta et al., J. Biol. Chem., 272:32321-32328 (1997)); and Ch6ST (human chondroitin-6-sulfotransferase; Fukuta et al., Biochim. Biophys. Acta, 1399:57-61 (1998)). The alignment revealed that the novel cDNA was homologous to other carbohydrate sulfotransferases, particularly I-GlcNAc6ST (FIG. 3C). The coding sequences of the novel cDNA and I-GlcNAc6ST were 90.6% identical at the nucleotide levels and 89.2% identical in the amino acid sequences, indicating that the novel cDNA encodes a carbohydrate sulfotransferase. The novel gene was designated CHST6 (carbohydrate sulfotransferase 6) and its product as corneal N-acetylglucosamine-6-sulfotransferase (C-GlcNAc6ST) in view of the expression of this transcript in cornea (see Example III).

BAC clone 483K2, which contains CHST6, was analyzed by PCR-based BAC screening (Research Genetics). BAC DNA was digested by restriction enzymes and the fragments were subcloned and sequenced as described in Example I. Sequencing analysis revealed that this gene is located about 30 kbp downstream of CHST5 in the same orientation (FIG. 3A and FIG. 3B). Both genes contain several introns in the 5′-untranslated region but do not contain introns in the coding region or the 3′-untranslated region. These two genes have regions that are highly homologous to each other not only in the coding region but also in the untranslated and upstream regions (FIG. 6A), indicating that CHST5 and CHST6 were created by gene duplication.

### EXAMPLE III

### Expression of Human Corneal GlcNAc6ST IS Absent from the Corneal Epithelium of a Mcd Type II Patient

This example demonstrates that the expression pattern of CHST6 mRNA in the cornea corresponds to the presence of sulfated keratan sulfate, and that CHST6 is not detectably expressed in corneal epithelial cells of a MCD type II patient.

The expression profile of CHST6 mRNA and the presence of sulfated keratan sulfate (sulfated KS) were analyzed in normal human cornea by in situ hybridization and immunohistochemistry (FIG. 4A through FIG. 4L). CHST6-specific DNA was amplified by PCR using CK71h-F1858 (5′-CACGAGGCCTGAACGGCTTCAC-3′; SEQ ID NO: 18) and CK71h-R1949 (5′-CGGGCCTAGCGCCTGCTACAAC-3′; SEQ ID NO: 19). This amplicon was cloned into the SmaI site of pGEM3Zf(+) (Promega; Madison, Wis.) and used to prepare RNA probes by DIG RNA Labeling Kit (Boehringer Mannheim; Indianapolis, Ind.). In situ hybridization was performed as described in Kawakami et al., Cancer Res. 57:2321-2324 (1997). Immunohistochemical detection of sulfated KS was performed using anti-sulfated KS monoclonal antibody (5D4; Seikagaku Co., Falmouth, Mass.) by the indirect method as described in Shiozawa et al., Gynecol. Obstet. Invest. 32:239-242 (1991). Hematoxylin was used for counterstaining.

Strong staining for sulfated KS was detected along the apical surface of the corneal epithelial cells (FIG. 4B) and in the endothelial cells (FIG. 4D). Sulfated KS was also found in the stroma (FIG. 4C). CHST6 transcripts also were detected in the upper layer of the corneal epithelial cells (FIG. 4F), stromal cells (FIG. 4G), and endothelial cells (FIG. 4H). These results indicate that CHST6 expression correlates with the presence of sulfated KS in human cornea. Analysis of mRNA distribution in other organs revealed that CHST6 transcripts were expressed in the spinal cord and the trachea. This tissue distribution differs from that of CHST5, which is predominantly expressed in the small intestine and the colon (Lee et al., supra, 1999).

Expression levels of CHST6 transcripts in a cornea from an MCD type II patient (M01 in FIG. 4D) were also examined. In situ hybridization analysis showed that CHST6 was not expressed at detectable levels in the epithelial cells (FIG. 4Q), but was expressed at normal levels in the stromal cells (FIG. 4R). This signal distribution was consistent with the staining pattern of sulfated KS detected by immunohistochemistry (FIG. 4N and FIG. 40). These findings demonstrate that the lack of CHST6 expression in corneal epithelial cells correlates with an undetectable amount of normal keratan sulfate in the same cells and can lead to characteristic opacities in the cornea of MCD patients (Nakazawa et al., J. Biol. Chem., 259:13751-13757 (1984)).

### EXAMPLE IV

### Mutations Associated with Mcd Type I

This example shows that MCD Type I occurs as a result of mutations that inactivate C-GlcNAc6ST.

Genomic PCR followed by direct-sequence analysis was carried out in searching for mutations in the coding regions of CHST6 of MCD patients and normal individuals. The coding region of CHST6 was amplified by PCR using the following primers: for the 5′-coding region, CK71h-intrn (5′-GCCCCTAACCGCTGCGCTCTC-3′; SEQ ID NO: 20) and Ck71h-R1180 (5′-GGCTTGCACACGGCCTCGCT-3′; SEQ ID NO: 21); for the middle coding region, CK71h-F1041 (5′-GACGTGTTTGATGCCTATCTGCCTTG-3′; SEQ ID NO: 22) and CK71h-R1674 (5′-CGGCGCGCACCAGGTCCA-3′; SEQ ID NO: 23); for the 3′-coding region, CK71h-F1355 (5′-CTCCCGGGAGCAGACAGCCAA-3′; SEQ ID NO: 24) and CK71h-R1953 (5′-CTCCCGGGCCTAGCGCCT-3′; SEQ ID NO: 25). Each PCR reaction was carried out in 25 μl according to the conditions described in Example I, with the exception of the cycled extension reaction lasting 45 seconds and the annealing temperatures changed to 55° C. for the middle coding region and 57° C. for the 5′- and 3′-coding regions. Amplified fragments were separated by electrophoresis in a 2% agarose gel, purified by QIAquick Gel Extraction Kit (Qiagen; Valencia, Calif.) and sequenced.

Mutations in the coding region of CHST6 (Table 1 and FIG. 5) were found in MCD type I patients. Four missense mutations identified in the patients' genomes were located at amino acid residues which are conserved among carbohydrate sulfotransferases (FIG. 3C). Two of these missense mutations, 203D→E and 211R→W, were located in the 3′-phosphate binding domain, which spans a Val¹⁹⁸ to Gln²¹³ and is important for sulfotransferase binding to adenosine 3′-phosphate 5′-phosphosulfate (PAPS) as a sulfate donor (Kakuta et al., Nature Struct. Biol., 4:904-908 (1997), and Kakuta et al., Trends Biochem. Sci., 23:129-130 (1998)). None of these mutations were present in 81 normal individuals. One frame shift mutation and a deletion mutation which lacks the entire coding region of CHST6 were also found in MCD type I patients. All of the mutations for MCD type I patients described in Table 1 were present in the homozygous state. The mutations found in type I patients demonstrate a loss of C-GlcNAc6ST function. These results indicate that C-GlcNAc6ST is required for production of sulfated KS and that inactivation of this gene is responsible for the MCD type I phenotype.

TABLE 1
Mutations of CHST6 identified in MCD patients

| | | Serum | | | | Same | |
|---|---|---|---|---|---|---|---|
| | | KS | | | | mutation in | |
| MCD | Patient | conc. | | | Mutation | control | |
| type | ID | (μg/ml) | Family | Mutation in DNA | in protein | chromosomes | Note |
| I | TO | <0.15 | | deletion of ORF | | 0/162 | Consanguineous |
| | | | | | | | marriage |
| I | SK | <0.15 | | 2T insertion | frameshift | 0/162 | Consanguineous |
| | | | | after 1106T | after 137A | | marriage |
| I | SY | <0.15 | | 1213A→G | 174K→R | 0/162 | |
| I | TM | <0.15 | A | 1301C→A | 203D→E | 0/162 | Consanguineous |
| | | | | | | | marriage |
| I | M03 | <0.15 | | 1301C→A | 203D→E | 0/162 | |
| I | MK | <0.15 | | 15120→A | 274E→K | 0/162 | Consanguineous |
| | | | | | | | marriage |
| I | SS | ND | | 1213A→G | 174K→R | 0/162 | |
| I | M09 | ND | | 1213A→G | 174K→R | 0/162 | |
| I | KS | ND | A | 1301C→A | 203D→E | 0/162 | Consanguineous |
| | | | | | | | marriage |
| I | M08 | ND | | 1323C→T | 211R→W | 0/162 | |
| II | #7 | 5.43 | B | 840C→A | 50R→A | 0/162 | Heterozygote of |
| | | | | | | | CHST6 |
| II | | | | replacement of 5′ | | 0/162 | by haplotype |
| | | | | region | | | analysis |
| II | #8 | 2.62 | B | 840C→A | 50R→C | 0/162 | Heterozygote of |
| | | | | | | | CHST6 |
| II | | | | replacement of 5′ | | 0/162 | by haplotype |
| | | | | region | | | analysis |
| II | #48 | 2.73 | B | 840C→A | 50R→C | 0/162 | Heterozygote of |
| | | | | | | | CHST6 |
| II | | | | replacement of 5′ | | 0/162 | by haplotype |
| | | | | region | | | analysis |
| II | #39 | 4.56 | | replacement of 5′ | | 0.162 | |
| | | | | region | | | |
| II | #54 | 3.03 | | replacement of 5′ | | 0/162 | |
| | | | | region | | | |
| II | M01 | 6.72 | C | deletion of 5′ | | 0/162 | Consanguineous |
| | | | | region | | | marriage |
| II | M24 | 3.70 | C | deletion of 5′ | | 0/162 | Consanguineous |
| | | | | region | | | marriage |
| II | M29 | 3.07 | C | deletion of 5′ | | 0/162 | Consanguineous |
| | | | | region | | | marriage |
| II | M30 | ND | | deletion of 5′ | | | |
| | | | | region | | | |

Normal 4.78 ± 1.49 (n = 10)
ND = not determined

To detect point mutation C1301A, PCR-RFLP analysis was used. The region flanking each point mutation was amplified by PCR using primers RFLP1 (5′-TGCTCTACCCGCTGCTCAGCGAC-3′; SEQ ID NO: 26) and RFLP2 (5′-CGGGAGCGCAGCACGGCCCCCGG-3′; SEQ ID NO: 27). PCR reactions were carried out as described in Example I, with the additional inclusion of a-³²P-dCTP in each reaction mixture and an annealing temperature of 57° C. After digestion with SmaI, amplified DNA fragments were separated on 15% polyacrylamide gels for 2 hours. The gel was stained by ethidium bromide and analyzed on an UV transilluminator.

### EXAMPLE V

### Mutations Associated with Mcd Type II

This example shows that MCD type II is correlated with altered or missing regulatory sequences.

MCD type II patients differ from MCD type I patients by having a detectable amount of serum sulfated KS (Table 1). Sulfated KS concentration in normal and patient serum was determined by ELISA as described in Thonar et al., Am. J. Ophthalmol. 102:561-569 (1986) and Thonar et al., Arthritis Rheum. 28:1367-1376 (1985), using bovine corneal keratan sulfate (Sigma; Saint Louis, Mo.) as a standard. Human serum was diluted sequentially by PBST-pH5.3 (phosphate buffered saline containing 0.05% Tween 20 and adjusted at pH5.3 by HCl). One hundred μl of each diluted sample was mixed with diluted 5D4 anti-sulfated KS monoclonal antibody in PBST-pH5.3 containing 1% BSA. After 1 hour incubation at room temperature, mixtures were transferred to a 96-well microtiter plate precoated with chondroitinase ABC treated bovine cartilage proteoglycan, and incubated for 1 hour at room temperature. Each well was washed three times with PBST-pH5.3 and 200 μl of peroxidase-conjugated goat anti-mouse IgG antibody diluted with PBST-pH5.3 containing 1% BSA was added. After incubation for 1 hour at room temperature, each well was washed three times with PBST-pH5.3. Peroxidase activity was measured using 1-Step ABTS (Pierce; Rockford, Ill.). The green color developed was measured by an ELISA microtiter plate reader at 405 nm.

No homozygous mutations in the coding region of CHST6 were detected in MCD type II patients. To probe for non-coding mutations in MCD type II patients, Southern blot analysis was carried out. Three micrograms of genomic DNA from patients and unaffected individuals were digested by SpeI, electrophoresed in a 0.7% agarose gel, and blotted onto a Nytran Plus filter (Schleicher & Schuell; Keene, N.H.). DNA probes were made by PCR according to the conditions described in Example I, using primers A3L114 (5′-TGCCCCCAGAAAAGAATCAAA-3′; SEQ ID NO: 28) and BamL142 (5′-TCCTCCCAAGTCCCTTGGAG-3′; SEQ ID NO: 29). Amplified probes were purified by QIAquick Gel Extraction Kit (Qiagen) and labeled with a-³²P-dCTP using Prime-It RmT kit (Stratagene). The blotted filter was hybridized with the probe in ExpressHyb hybridization solution (Clontech), according to the methods recommended by the manufacturer. After washing with 1× SSC-0.1% SDS at 50° C. for 1 hour, the filter was exposed to X-ray film with an intensifying screen at −80° C. for 5 days.

Southern blot analyses indicated DNA rearrangements in the upstream region of CHST6. One rearrangement replaced a 2.5 kbp region located upstream of CHST6 exon 1, with a region that was originally located upstream of CHST5 exon 1 (FIG. 6A). Patients #39 and #54 had this replacement mutation in both alleles (FIG. 6B).

Genomic PCR analysis identified the junctions of this replacement mutation. Replacement mutations found in the upstream region 2 of CHST6 were detected by PCR using following primers: for the normal homologous region A, Fl (5′-CCACAGAAGGAAGGACAGAGTAAATGAA-3′; SEQ ID NO: 30) and R1 (5′-TTCCCTTTACTATTATAAAAATGCTGCTAATG-3′; SEQ ID NO: 31); for the replaced homologous region A, F1 and R1M (5′-TGCTGAATGGCTAACTGAAGGAATACTATAC-3′; SEQ ID NO: 32); for the normal homologous region B, F2 (5′-CATATCCTGTCTGGCCTAAACCTTAGTTTAC-3′; SEQ ID NO: 33) and R2 (5′-GGGCACAGACAGAGGGAAAAACC-3′; SEQ ID NO: 34); for the replaced homologous region B, F2M (5′-GGCCAAGTTCAGGTCAGCTTCCA-3′; SEQ ID NO: 35) and R2. The same reaction cycles were used as described in Example I, except the annealing temperature was 55° C.

PCR reactions using F1-R1 and F2-R2 primer pairs, which amplify upstream regions of CHST6 in normal individuals, yielded no detectable bands for patient #39 and #54. However, PCR primer sets F1/R1M and F2M/R2, which do not amplify normal genomic DNA, produced DNA amplicons when #39 and #54 genomic DNAs were used as templates (FIG. 6B). These results indicate that the DNA region which contains the R1 and F2 sequences was replaced by the region spanning the R1M and F2M sequences (FIG. 6A). Since the flanking sequences of both these regions are highly homologous to each other (region A and region B in FIG. 6A), this replacement mutation found in patients #39 and #54 can be a result of homologous recombination.

This replacement was also found in another unrelated MCD type II family (FIG. 6C). Haplotype analysis indicated that the patients in this family have different mutations in each allele. Specifically, genomic PCR and direct sequence analyses revealed that these type II patients had a replacement mutation upstream of CHST6 on the maternal allele and a missense mutation in the coding region on the paternal allele (FIG. 6C). The missense mutation was classified as a type I mutation because the mutation, R50C (nucleotide replacement C840A), was located in a conserved domain for 5′ PAPS binding (Kakuta et al., Nature Struct. Biol. 4:904-908 (1997), and Kakuta et al., Trends Biochem. Sci. 23:129-130 (1998); FIG. 6C) and is likely to affect C-GlcNAc6ST activity in a manner similar to other type I mutations.

PCR-RFLP analysis was carried out to detect the R50C missense mutation using the above-described PCR-RFLP method with primers CK71h-F781 (5′-AGACCTTCCTCCTCCTCTTTCTGGTT-3′; SEQ ID NO: 36) and RFLP3 (5′-TTGGCCCACGAAGGACGAGCCCGGGC-3′; SEQ ID NO: 37), and by digestion with KasI. Replacement PCR and PCR-RFLP analyses showed that these patients (FIG. 6C) had heterozygote compounds with mutations classified as type I and type II in different alleles. Because these patients had measurable serum sulfated KS levels characteristic of MCD type II, these results indicate that the MCD type II phenotype is dominant over the type I phenotype. Previous studies have also reported the existence of MCD type II patients in type I families, which is consistent with these findings (Liu et al., Am. J. Hum. Genet. 63:912-917 (1998); Klintworth et al., Am. J. Ophthalmol. 124:9-18 (1997); and Liu et al., Br. J. Ophthalmol. 82:241-244 (1998)).

Another DNA rearrangement was found in a large MCD type II family (FIG. 6D). Southern blot analysis was carried out as described above, and PCR analysis was carried out using the method described above for replacement PCR analysis. Results revealed that a large DNA region including CHST5 and the upstream region of CHST6 is missing in these type II patients. Since F2M/R2 primers amplified genomic DNA from both the patients and unaffected heterozygous family individuals who have the same haplotype as type II patients, the disease-causative mutation in this family is a 40 kbp deletion spanning from the homologous region B that is upstream of CHST5 to the region B of CHST6 (see FIG. 6A).

All type II mutations were found in the upstream region of CHST6, which can contain gene regulatory elements that affect transcription of CHST6. These results and the histological results of Example III demonstrate that MCD type II mutations promote loss of CHST6 expression in the cornea, while expression in other KS-rich tissues such as cartilage is not affected. This explains the marked difference in serum sulfated KS levels between MCD type I and II, while the clinical phenotypes of MCD type I and II are indistinguishable.

### EXAMPLE VI

This example demonstrates that human corneal GlcNAc6St and murine I-GlcNAc6ST catalyze sulfation of KS in HeLa cells.

The ability of C-GlcNAc6ST, the observed C-GlcNAc6ST mutants, and several sulfotransferases homologous to C-GlcNAc6ST to catalyze sulfur transfer to KS in HeLa cells was tested. cDNA encoding human keratan sulfate galactose-6-sulfotransferase (KSG6) and murine intestinal N-acetylglucosamine-6-sulfotransferase (mI-GlcNAc6ST) were obtained by PCR from human and murine genomic DNA, respectively, and were each cloned into pcDNA3.1, as was I-GlcNAc6ST, C-GlcNAc6ST, and C-GlcNAc6ST mutants 50R→C, 174K→R, 203D→E, 211R→W, 217A→T and 274E→K.

Sulfotransferase encoding vectors were then transformed into HeLa cells by lipofection or LipofectAmine PLUS (GIBCO-BRL). Transformed HeLa cells were grown in duplicate in DMEM media containing unsulfated KS and media lacking any form of KS. After two days, staining for sulfated KS was carried out using anti-sulfated KS antibody 5D4 as described above. The results demonstrate that, in HeLa cells, related human proteins KSG6 and I-GlcNAc6ST do not catalyze sulfation of KS, whereas the human corneal GlcNAc6ST (SEQ ID NO: 2) and murine I-GlcNAc6ST (SEQ ID NO: 5) do produce sulfated keratan sulfate (see Table 2). Additionally, all variant forms of human corneal GlcNAc6ST obtained from in MCD type I and II patients lacked catalytic activity.

TABLE 2
Activity of wild type and mutant sulfotransferases

| Sulfotransferase gene product | Sulfated KS level |
|---|---|
| None | − |
| KSG6ST | − |
| I-GlcNAc6ST | − |
| C-GlcNAc6ST | +++ |
| mI-GlcNAc6ST | +++ |
| R50C | − |
| K174R | − |
| D203E | − |
| R211W | − |
| A217T | − |
| E274K | − |

The corneally-expressed GlcNAc6ST of mice and human demonstrate specific catalytic KS sulfotransferase activity in HeLa cells. This result is supported by the amino acid residues conserved between mouse I-GlcNAc6ST and human C-GlcNAc6ST, but not in human I-GlcNAc6ST, as demonstrated by amino acid sequence alignment calculated using ClustalW (FIG. 2).

All journal article, reference, and patent citations provided above, in parentheses or otherwise, whether previously stated or not, are incorporated herein by reference.

Although the invention has been described with reference to the examples above, it should be understood that various modifications can be made without departing from the spirit of the invention. Accordingly, the invention is limited only by the following claims.

We claim:

1. An isolated nucleic acid molecule, comprising a sequence encoding a corneal N-acetylglucosamine-6-sulfotransferase (GlcNAc6ST) or active fragment thereof, wherein said GlcNAc6ST or active fragment thereof catalyzes the sulfation of keratan sulfate.

2. The isolated nucleic acid molecule of claim 1, wherein said GlcNAc6ST has substantially the amino acid sequence of SEQ ID NO: 2.

3. The isolated nucleic acid molecule of claim 2, comprising a nucleic acid sequence encoding SEQ ID NO: 2.

4. The isolated nucleic acid molecule of claim 3, comprising SEQ ID NO: 1.

5. The isolated nucleic acid molecule of claim 1, wherein said sulfation of keratan sulfate produces sulfated keratan sulfate immunoreactive with antibody 5D4.

6. The isolated nucleic acid molecule of claim 1, wherein said sulfation of keratan sulfate produces sulfated keratan sulfate hydrolyzable by keratanase.

7. A vector, comprising a nucleic acid molecule encoding a corneal N-acetylglucosamine-6-sulfotransferase (GlcNAc6ST), wherein said GlcNAc6ST or active fragment thereof catalyzes sulfation of keratan sulfate.

8. The vector of claim 7, which is a mammalian expression vector.

9. Host cells, comprising the vector of claim 7.

10. An oligonucleotide, comprising a nucleotide sequence having at least 10 contiguous nucleotides of a nucleic acid molecule selected from the group consisting of SEQ ID NO: 1 and SEQ ID NO: _, or a nucleotide sequence complementary thereto, provided that the oligonucleotide sequence does not consist of a sequence of GenBank accession number AI824100.

11. The oligonucleotide of claim 10, having at least 15 contiguous nucleotides of a nucleic acid molecule selected from the group consisting of SEQ ID NO: 1 and SEQ ID NO: , or a nucleotide sequence complementary thereto.

12. An isolated polypeptide, comprising an amino acid sequence encoding a corneal N-acetylglucosamine-6-sulfotransferase (GlcNAc6ST) or active fragment thereof, wherein said GlcNAc6ST or active fragment thereof catalyzes sulfation of keratan sulfate.

13. The isolated polypeptide of claim 12, wherein said GlcNAc6ST has substantially the amino acid sequence of SEQ ID NO: 2.

14. The isolated polypeptide of claim 13, wherein said GlcNAc6ST has the amino acid sequence SEQ ID NO: 2.

15. Substantially purified antibody material that specifically binds a corneal N-acetylglucosamine-6-sulfotransferase (GlcNAc6ST), wherein said GlcNAc6ST catalyzes sulfation of keratan sulfate.

16. The substantially purified antibody material of claim 15, which specifically binds a GlcNAc6ST having the amino acid sequence SEQ ID NO: 2.

17. The substantially purified antibody material of claim 15, which is monoclonal antibody material.

18. A method of treating a subject with macular corneal dystrophy, comprising administering to said subject an effective amount of an agent that increases expression or activity of a N-acetylglucosamine-6-sulfotransferase (GlcNAc6ST), whereby the amount of sulfated keratan sulfate in the cornea of said subject is elevated.

19. The method of claim 18, wherein said subject has type I macular corneal dystrophy.

20. The method of claim 18, wherein said subject has type II macular corneal dystrophy.

21. The method of claim 18, wherein said GlcNAc6ST is endogenous GlcNAc6ST.

22. The method of claim 18, wherein said agent is a nucleic acid molecule encoding a GlcNAc6ST, or active fragment thereof that catalyzes the sulfation of keratan sulfate.

23. The method of claim 22, wherein said GlcNAc6ST is selected from the group consisting of human GlcNAc6ST and murine GlcNAc6ST.

24. The method of claim 22, wherein said nucleic acid molecule encodes substantially the amino acid sequence of SEQ ID NO:2.

25. The method of claim 24, wherein said nucleic acid molecule comprises the sequence of SEQ ID NO:1.

26. The method of claim 22, wherein said agent is a GlcNAc6ST, or active fragment thereof, that catalyzes the sulfation of keratan sulfate.

27. The method of claim 26, wherein said GlcNAc6ST is selected from the group consisting of human GlcNAc6ST and murine GlcNAc6ST.

28. The method of claim 26, wherein said GlcNAc6ST has substantially the amino acid sequence of SEQ ID NO:2.

29. The method of claim 28, wherein said GlcNAc6ST comprises the sequence of SEQ ID NO:2.

30. The method of claim 18, wherein said agent increases transcription of a GlcNAc6ST that catalyzes the sulfation of keratan sulfate.

31. The method of claim 30, wherein said GlcNAc6ST is selected from the group consisting of human GlcNAc6ST and murine GlcNAc6ST.

32. The method of claim 30, wherein said agent selectively increases transcription of GlcNAc6ST in the cornea of said subject.

33. A method of treating a subject with macular corneal dystrophy, comprising the steps of:
(a) administering in vitro to primary, explanted corneal cells an effective amount of an agent that increases expression or activity of a N-acetylglucosamine-6-sulfotransferase; and
(b) introducing said cells into the cornea of said subject, whereby the amount of sulfated keratan sulfate in the cornea of said subject is elevated.

34. A method of monitoring therapeutic efficacy in a subject being treated for macular corneal dystrophy, comprising the steps of:
(a) obtaining a test sample from said subject;
(b) determining a sample level of expression or activity of N-acetylglucosamine-6-sulfotransferase (GlcNAc6ST) in said test sample; and
(c) comparing said sample level to a reference level from said subject; whereby an increase in said sample level relative to said reference level is indicative of productive therapy.

35. The method of claim 34, wherein said sample level is measured using a nucleic acid molecule that specifically hybridizes to a nucleotide sequence selected from the group consisting of SEQ ID NO: 1 and SEQ ID NO: 38.

36. The method of claim 34, wherein said sample level is measured using an antibody that specifically binds GlcNAc6ST.

37. A method of determining susceptibility to macular corneal dystrophy (MCD) in an individual, comprising determining the presence or absence in an individual of a MCD-associated allele linked to a corneal N-acetylglucosamine-6-sulfotransferase locus, wherein the presence of said MCD-associated allele indicates susceptibility to MCD in said individual.

38. The method of claim 37, wherein said macular corneal dystrophy is type I macular corneal dystrophy.

39. The method of claim 37, wherein said macular corneal dystrophy is type II macular corneal dystrophy.

40. The method of claim 37, wherein said MCD-associated allele is within a corneal N-acetylglucosamine-6-sulfotransferase gene.

41. The method of claim 40, wherein said MCD-associated allele is within a corneal N-acetylglucosamine-6-sulfotransferase coding region.

42. The method of claim 41, wherein said MCD-associated allele is a mutation of SEQ ID NO:1 selected from the group consisting of deletion of the entire open reading frame, insertion of two T's after 1106T, 1213A→G, 1301C→A, 1512G→A, 1323C→T, and 840C→A.

43. The method of claim 41, wherein said MCD-associated allele is within the region coding the 3′-phosphate binding domain of corneal N-acetylglucosamine-6-sulfotransferase.

44. The method of claim 43, wherein said MCD-associated allele is a nucleotide sequence encoding an amino acid mutation of SEQ ID NO:2 selected from the group consisting of 203D→E and 211R→W.

45. The method of claim 40, wherein said MCD-associated allele is within a corneal N-acetylglucosamine-6-sulfotransferase 5′ regulatory region.

46. The method of claim 45, wherein said MCD-associated allele is selected from the group consisting of replacement of a 51 region of CHST6 with a 5′ region of CHST5 and deletion of a 5′ region of CHST6.
